SEQUENCE LISTING (6 sequences)

SEQ ID NO: 1 - DNA, 29 bp, synthetic construct
  source 1..29: mol_type = other DNA; note = forward primer
ctcgtttttcaactactccacaaatttct

SEQ ID NO: 2 - DNA, 26 bp, synthetic construct
  source 1..26: mol_type = other DNA; note = reverse primer
gtgtcatgcacaaagtagatgtccta

SEQ ID NO: 3 - DNA, 16 bp, synthetic construct
  source 1..16: mol_type = other DNA; note = (FAM)-labeled probe
ctgacttgccaaaact

SEQ ID NO: 4 - DNA, 19 bp, synthetic construct
  source 1..19: mol_type = other DNA; note = forward primer
aagccgaagaacaccatcc

SEQ ID NO: 5 - DNA, 18 bp, synthetic construct
  source 1..18: mol_type = other DNA; note = reverse primer
agcacccccacaacatga

SEQ ID NO: 6 - DNA, 16 bp, synthetic construct
  source 1..16: mol_type = other DNA; note = UPL probe #87
ctgacttgccaaaact

(19) United States
(12) Patent Application Publication
(10) Pub. No.: US 2026/0365979 A1
(43) Pub. Date: Nov. 19, 2026

(54) METHODS FOR GENERATING GENETICALLY MODIFIED CYTOKINE INDUCED KILLER (CIK) CELLS

(71) Applicant: FONDAZIONE MATILDE TETTAMANTI E MENOTTI DE MARCHI ONLUS, Monza (IT)

(72) Inventors: Irina TCHEREPANOVA, Chapel Hill, NC (US); Melissa ADAMS, Chapel Hill, NC (US); John KRISKO, Menane, NC (US); Mark DEBENEDETTE, Durham, NC (US); Charles NICOLETTE, Durham, NC (US); Sarah TETTAMANTI, Monza (IT); Giuseppe GAIPA, Monza (IT); Andrea BIONDI, Monza (IT); Laura CONTRERAS-RUIZ, Apex, NC (US)

(21) Appl. No.: 19/136,351

(22) PCT Filed: Dec. 7, 2023
(86) PCT No.: PCT/IB2023/062377

§ 371 Date: Jun. 6, 2025

Related U.S. Application Data
(60) Provisional application No. 63/386,454, filed on Dec. 7, 2022.

(51) Int. Cl. C12N 5/0783; A61K 40/11; A61K 40/31; A61K 40/42

(52) U.S. Cl. CPC C12N 5/0638; A61K 40/11; A61K 40/31; A61K 40/4211; C12N 2501/2302; C12N 2501/515; C12N 2506/11; C12N 2510/00

(57) ABSTRACT

The present disclosure provides methods of generating genetically modified cytokine induced killer (CIK) cells, comprising the sequential steps of: (a) culturing a population of mononuclear cells in culture medium comprising at least one differentiating agent to induce differentiation of the mononuclear cells in a cell culture into Committed CIK Precursor cells; (b) adding at least one stimulating agent and at least one expanding agent to the cell culture; and (c) expanding the cells from the cell culture to obtain a cell population comprising Committed CIK Precursor cells, transfecting the cell population comprising the Committed CIK Precursor cells with one or more nucleic acids to produce genetically modified Committed CIK Precursor cells, and expanding the genetically modified Committed CIK Precursor cells in culture medium to produce the genetically modified CIK cells; or (c) expanding the cells from the cell culture to obtain a cell population comprising Committed CIK Precursor cells and transfecting the cell population comprising the Committed CIK Precursor cells with one or more nucleic acids; (d) formulating transfected cells comprising the Committed CIK Precursor cells for administration to a subject in need thereof; and (e) administering the transfected cells to the subject, wherein transfected Committed CIK Precursor cells are expanded to produce the genetically modified CIK cells in the subject; wherein steps (a), (b) and (c) are performed in the absence of non-irradiated or irradiated feeder cells.

### CROSS REFERENCE TO RELATED APPLICATIONS

This application claims the benefit of the filing date of U.S. Provisional Patent Application No. 63/386,454 filed Dec. 7, 2022, the contents of which are incorporated herein by reference in its entirety.

### FIELD OF THE DISCLOSURE

The present disclosure provides methods of generating genetically modified cytokine induced killer (CIK) cells, comprising the sequential steps of: (a) culturing a population of mononuclear cells in culture medium comprising at least one differentiating agent to induce differentiation of the mononuclear cells in a cell culture into Committed CIK Precursor cells; (b) adding at least one stimulating agent and at least one expanding agent to the cell culture; and (c) expanding the cells from the cell culture to obtain a cell population comprising Committed CIK Precursor cells, transfecting the cell population comprising the Committed CIK Precursor cells with one or more nucleic acids to produce genetically modified Committed CIK Precursor cells, and expanding the genetically modified Committed CIK Precursor cells in culture medium to produce the genetically modified CIK cells; or (c) expanding the cells from the cell culture to obtain a cell population comprising Committed CIK Precursor cells and transfecting the cell population comprising the Committed CIK Precursor cells with one or more nucleic acids; (d) formulating transfected cells comprising the Committed CIK Precursor cells for administration to a subject in need thereof; and (e) administering the transfected cells to the subject, wherein transfected Committed CIK Precursor cells are expanded to produce the genetically modified CIK cells in the subject; wherein steps (a), (b) and (c) are performed in the absence of non-irradiated or irradiated feeder cells.

### BACKGROUND OF THE DISCLOSURE

Cytokine-induced killer (CIK) cells are cytotoxic T cells, which have both NK and T cell properties. The qualifier “cytokine-induced killer” indicates that they are generated via administration of cytokines during in vitro culture (Arafar, A., Biomedical Research and Therapy; 1(2):71-77 (2014); Linn, Y. C., and Hui, K. M., J Biomed Biotechnol.; 2010:435745 (2010)). Cells which have the most potential effector function in CIK culture coexpress CD3 and CD56 surface molecules; possess a potent, HLA-unrestricted tumor-killing ability; and significantly reduced alloreactivity. Thus, CIK cells are capable of killing a wide range of tumor cells, and hold great promise in the field of adoptive immunotherapy for cancer treatment and virus infections.

Accordingly, there is a need to develop the methods of improved generation of genetically modified CIK cells in vitro, in order to obtain a population of effector cells with immunotherapeutic activity.

### BRIEF SUMMARY OF THE DISCLOSURE

One aspect of the present disclosure is directed to methods of generating genetically modified cytokine induced killer (CIK) cells, comprising the sequential steps of: (a) culturing a population of mononuclear cells in culture medium comprising at least one differentiating agent to induce differentiation of the mononuclear cells in a cell culture into Committed CIK Precursor cells; (b) adding at least one stimulating agent and at least one expanding agent to the cell culture; and (c) expanding the cells from the cell culture to obtain a cell population comprising Committed CIK Precursor cells, transfecting the cell population comprising the Committed CIK Precursor cells with one or more nucleic acids to produce genetically modified Committed CIK Precursor cells, and expanding the genetically modified Committed CIK Precursor cells in culture medium to produce the genetically modified CIK cells; wherein steps (a), (b) and (c) are performed in the absence of non-irradiated or irradiated feeder cells.

One aspect of the present disclosure is directed to methods of generating genetically modified cytokine induced killer (CIK) cells, comprising the sequential steps of: (a) culturing a population of mononuclear cells in culture medium comprising at least one differentiating agent to induce differentiation of the mononuclear cells in a cell culture into Committed CIK Precursor cells; (b) adding at least one stimulating agent and at least one expanding agent to the cell culture; (c) expanding the cells from the cell culture to obtain a cell population comprising Committed CIK Precursor cells and transfecting the cell population comprising the Committed CIK Precursor cells with one or more nucleic acids; (d) formulating transfected cells comprising the Committed CIK Precursor cells for administration to a subject in need thereof; and (e) administering the transfected cells to the subject, wherein transfected Committed CIK Precursor cells are expanded to produce the genetically modified CIK cells in the subject; wherein steps (a), (b) and (c) are performed in the absence of non-irradiated or irradiated feeder cells.

One aspect of the present disclosure is directed to methods of generating genetically modified cytokine induced killer (CIK) cells, comprising the sequential steps of: (a) culturing peripheral blood mononuclear cells (PBMCs) in culture medium comprising at least one differentiating agent to induce differentiation of the PBMCs in a cell culture into Committed CIK Precursor cells; (b) adding at least one stimulating agent and at least one expanding agent to the cell culture; and (c) expanding the cells from the cell culture to obtain a cell population comprising Committed CIK Precursor cells, transfecting the cell population comprising the Committed CIK Precursor cells with one or more nucleic acids to produce genetically modified Committed CIK Precursor cells, and expanding the genetically modified Committed CIK Precursor cells in culture medium to produce the genetically modified CIK cells; wherein steps (a), (b) and (c) are performed in the absence of non-irradiated or irradiated feeder cells; and wherein the PBMCs were previously cryopreserved and thawed before culturing in step (a).

One aspect of the present disclosure is directed to methods of generating genetically modified cytokine induced killer (CIK) cells, comprising the sequential steps of: (a) culturing peripheral blood mononuclear cells (PBMCs) in culture medium comprising at least one differentiating agent to induce differentiation of the PBMCs in a cell culture into Committed CIK Precursor cells; (b) adding at least one stimulating agent and at least one expanding agent to the cell culture; (c) expanding the cells from the cell culture to obtain a cell population comprising Committed CIK Precursor cells and transfecting the cell population comprising the Committed CIK Precursor cells with one or more nucleic acids;
(d) formulating transfected cells comprising the Committed CIK Precursor cells for administration to a subject in need thereof; and (e) administering the transfected cells to the subject, wherein transfected Committed CIK Precursor cells are expanded to produce the genetically modified CIK cells in the subject; wherein steps (a), (b) and (c) are performed in the absence of non-irradiated or irradiated feeder cells; and wherein the PBMCs were previously cryopreserved and thawed before culturing in step (a).

In some aspects, the stimulating agent and the expanding agent are added to the cell culture approximately 8 to 48 hours after initiating step (a). In some aspects, the stimulating agent and the expanding agent are added to the cell culture approximately 18 to 24 hours after initiating step (a).

In some aspects, the Committed CIK Precursor cells are transfected with one or more nucleic acids approximately 18 to 48 hours after initiating step (b). In some aspects, the Committed CIK Precursor cells are transfected with one or more nucleic acids approximately 18 to 24 hours after initiating step (b).

In some aspects, the methods disclosed herein further comprise (d) replacing a portion of the culture medium with a fresh culture medium comprising at least one expanding agent approximately 8 to 96 hours after initiating step (c). In some aspects, the methods disclosed herein further comprise (d) replacing a portion of the culture medium with a fresh culture medium comprising at least one expanding agent approximately 18 to 24 hours after initiating step (c).

In some aspects, the methods disclosed herein further comprise (e) passaging and culturing transfected cells in culture medium comprising at least one expanding agent approximately 8 to 96 hours after initiating step (d). In some aspects, the methods disclosed herein further comprise (e) passaging and culturing transfected cells in culture medium
comprising at least one expanding agent approximately 72 to 96 hours after initiating step (d).

In some aspects, after step (e) the transfected cells in culture medium are transferred to a cell culture bag, a cell culture flask, or a culture device. In some aspects, the transfected cells in culture medium are transferred to the cell culture bag. In some aspects, the culture device is a bioreactor.

In some aspects, the transfected cells are expanded by culturing the transfected cells in culture medium comprising at least one expanding agent about every 2 to 3 days or about every 3 to 4 days until about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, or about 28 days after initiating step (a).

In some aspects, the transfected cells are expanded until about 10 days after initiating step (a). In some aspects, the transfected cells are expanded until about 14 days after initiating step (a). In some aspects, the transfected cells are expanded until about 17 days after initiating step (a). In some aspects, the transfected cells are expanded until about 21 days after initiating step (a). In some aspects, the transfected cells are expanded until about 28 days after initiating step (a).

In some aspects, the methods disclosed herein further comprise (f) isolating the cells from the cell culture to obtain a cell population comprising the genetically modified CIK cells.

In some aspects, the transfected cells are cultured to obtain a cell population comprising the genetically modified CIK cells.

In some aspects, the methods disclosed herein further comprise the step of freezing the genetically modified CIK cells.

In some aspects, the genetically modified CIK cells disclosed herein express one or more T cell receptors (TCR-CIK), chimeric antigen receptors (CAR-CIK), a genetically modified adhesion molecule, a genetically modified ligand, a genetically modified cytokine receptor, a genetically modified chemokine receptor, a genetically modified cytokine, a genetically modified chemokine, an enzyme, or a checkpoint inhibitor.

In some aspects, the genetically modified CIK cells disclosed herein express a prodrug converting enzyme, an IgG-degrading enzyme of S. pyogenes (IdeS), a consensus variant 1 (CV1) protein, Intercellular Adhesion Molecule 1 (ICAM-1), CD137L, OX40L, CD70, IL-15 receptor, CCR5, CCR4, CD25, CD122, CD132, C-X-C chemokine receptor type 4 (CXCR4), IL-15, IL-18, IL-21 IL-23 IL-33, IL-1a, IL-1b, matrix metalloproteinase (MMP), heparinase, an anti-PD-1 antibody or antigen binding fragment thereof, an anti-T cell immunoglobulin and mucin-domain containing-3 (TIM-3) antibody or antigen binding fragment thereof, IL-3 zetakine, or any combination thereof.

In some aspects, the population of mononuclear cells disclosed herein is selected from the group consisting of: umbilical cord blood derived mononuclear cells, peripheral blood mononuclear cells (PBMCs), bone marrow derived mononuclear cells, lymphocytes, monocytes, dendritic cells, macrophages, T cells, naive T cells, memory T cells, natural killer cells, hematopoietic stem cells, pluripotent embryonic stem cells, induced pluripotent stem cells, and any combination thereof.

In some aspects, the mononuclear cells are umbilical cord blood derived mononuclear cells. In some aspects, the mononuclear cells are PBMCs. In some aspects, the PBMCs were previously cryopreserved and thawed before culturing in step (a).

In some aspects, the mononuclear cells are from a human leukocyte antigen (HLA) matched donor.

In some aspects, the differentiating agent is selected from the group consisting of: IFN-γ, IL-4, IL-5, IL-7, IFN-α, IL-10, IL-12, IL-13, IL-6, IL-15, IL-17, IL-18, IL-21, IL-22, IL-23, IL-27, IL-1β, TGF-β, GM-CSF, CCL3, CCL4, CCL5, CCL17, CCL21, and any combination thereof. In some aspects, the differentiating agent is IFN-γ.

In some aspects, the differentiating agent is added in an amount of about 10 U/ml to about 10000 U/ml. In some aspects, the differentiating agent is added in an amount of about 1000 U/ml.

In some aspects, the stimulating agent is selected from the group consisting of: an anti-CD3 antibody, an anti-TCR antibody, an anti-CD28 antibody, an anti-CD137 antibody, an anti-CD134 antibody, an anti-CD27 antibody, an anti-ICAM-1 antibody, an anti-CD3/CD28-coated beads, a superantigen, phytohaemaglutinin (PHA), phorbol 12-myristate 13-acetate (PMA), ionomycin, and any combination thereof.

In some aspects, the stimulating agent is an anti-CD3 antibody. In some aspects, the anti-CD3 antibody is an OKT3 antibody.

In some aspects, the stimulating agent is added in an amount of about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 31 ng/ml, about 32 ng/ml, about 33 ng/ml, about 34 ng/ml, about 35 ng/ml, about 36 ng/ml, about 37 ng/ml, about 38 ng/ml, about 39 ng/ml, about 40 ng/ml, about 41 ng/ml, about 42 ng/ml, about 43 ng/ml, about 44 ng/ml, about 45 ng/ml, about 46 ng/ml, about 47 ng/ml, about 48 ng/ml, about 49 ng/ml, about 50 ng/ml, about 51 ng/ml, about 52 ng/ml, about 53 ng/ml, about 54 ng/ml, about 55 ng/ml, about 56 ng/ml, about 57 ng/ml, about 58 ng/ml, about 59 ng/ml, about 60 ng/ml, about 61 ng/ml, about 62 ng/ml, about 63 ng/ml, about 64 ng/ml, about 65 ng/ml, about 66 ng/ml, about 67 ng/ml, about 68 ng/ml, about 69 ng/ml, about 70 ng/ml, about 71 ng/ml, about 72 ng/ml, about 73 ng/ml, about 74 ng/ml, about 75 ng/ml, about 76 ng/ml, about 77 ng/ml, about 78 ng/ml, about 79 ng/ml, about 80 ng/ml, about 81 ng/ml, about 82 ng/ml, about 83 ng/ml, about 84 ng/ml, about 85 ng/ml, about 86 ng/ml, about 87 ng/ml, about 88 ng/ml, about 89 ng/ml, about 90 ng/ml, about 91 ng/ml, about 92 ng/ml, about 93 ng/ml, about 94 ng/ml, about 95 ng/ml, about 96 ng/ml, about 97 ng/ml, about 98 ng/ml, about 99 ng/ml, or about 100 ng/ml.

In some aspects, the stimulating agent is added in an amount of about 50 ng/ml.

In some aspects, the expanding agent is selected from the group consisting of: IL-2, IL-4, IL-7, IL-9, IL-15, IL-18, IL-21, and any combination thereof. In some aspects, the expanding agent is IL-2.

In some aspects, the expanding agent is added in an amount of about 10 U/ml to about 1000 U/ml. In some aspects, the expanding agent is added in an amount of about 300 U/ml.

In some aspects, the cell population comprising the Committed CIK Precursor cells is transfected using electroporation.

In some aspects, the electroporation in performed in an isotonic buffer. In some aspects, the electroporation in performed in CoStorSol, Opti-MEM, or Lonza isotonic buffer.

In some aspects, the transfection is selected from the group consisting of: a non-viral transfer of one or more nucleic acids encoding an antigen receptor, a chimeric antigen receptor, a T cell receptor, a suicide gene, a gene encoding an inducible caspase 9 system, and any combination thereof into the cell population comprising the Committed CIK Precursor cells in the cell culture.

In some aspects, the non-viral transfer of nucleic acids comprises the use of the group consisting of: a transposon-based integration system, Zn-finger nucleases, integrases, transcription activator-like effectors, clustered regularly interspaced short palindromic repeats (CRISPR), sequence-specific recombinase systems able to integrate nucleic acids by recombination between attachment sites, and any combination thereof.

In some aspects, the transposon-based system is a Sleeping Beauty (SB) transposon-based system. In some aspects, the SB transposon-based system comprises the use of Sleeping Beauty transposase SB100X.

In some aspects, one or more nucleic acids encode T cell receptors, chimeric antigen receptors, a cell adhesion molecule, a ligand, a cytokine receptor, a chemokine receptor, a cytokine, a chemokine, an enzyme, or a checkpoint inhibitor.

In some aspects, one or more nucleic acids encode a prodrug converting enzyme, an IgG-degrading enzyme of S. pyogenes (IdeS), a consensus variant 1 (CV1) protein, Intercellular Adhesion Molecule 1 (ICAM-1), CD137L, OX40L, CD70, IL-15 receptor, CCR5, CCR4, CD25, CD122, CD132, C-X-C chemokine receptor type 4 C-X-C chemokine receptor type 4 (CXCR4), IL-15, IL-18, IL-21 IL-23 IL-33, IL-1a, IL-1b, matrix metalloproteinase (MMP), heparinase, an anti-PD-1 antibody or antigen binding fragment thereof, an anti-T cell immunoglobulin and mucin-domain containing-3 (TIM-3) antibody or antigen binding fragment thereof, IL-3 zetakine, or any combination thereof.

In some aspects, the prodrug converting enzyme is carboxypeptidase G2 (CPG2) or β-lactamase.

In some aspects, the chimeric antigen receptors are specific for CD19, CD123, TIM-3, C-type lectin-like molecule-1 (CLL-1), CD70, mucin 1 (MUC-1), CD20, CD22, B-cell activating factor receptor (BAFFR), CD23, cytokine receptor like factor 2 (CRLF2), CD79b, CD79d, CD7, CD43, CD5, CD25, Lewis Y (LeY), natural killer group 2 member D (NKG2D), receptor tyrosine kinase like orphan receptor 1 (ROR1), receptor tyrosine kinase like orphan receptor 2 (ROR2), Wilms' tumor 1 (WT1), CD44 variant 6 (CD44v6), CD33, CD3ε, human epidermal growth factor receptor 2 (Her2), epidermal growth factor receptor (EGFR), epidermal growth factor receptor variant III (EGFRvIII), CA125, CD138, prostate-specific membrane antigen (PSMA), B7 homolog 3 protein (B7-H3), CD30, disialoganglioside GD-2, disialoganglioside GD-3, CD171, mesothelin (MSLN), ephrin type-A receptor 2 (EphA2), carcinoembryonic antigen (CEA), vascular endothelial growth factor receptor (VEGFR), IL13Rα2, prostate stem cell antigen (PSCA), epithelial cellular adhesion molecule (EpCAM), chondroitin sulfate proteoglycan 4 (CSPG4), folate receptor alpha (FRα), fibroblast activation protein (FAP), carbonic anhydrase IX (CAIX), B-cell maturation antigen (BCMA), signaling lymphocytic activation molecule family member 7 (SLAM7), CD126, mesenchymal-epithelial transition factor (c-MET), AXL receptor tyrosine kinase (AXL), CD133, tumor endothelial marker 8 (TEM8), tumor-associated calcium signal transducer 2 (TROP2), programmed death-ligand 1 (PD-L1), delta-like ligand 3 (DLL3), melanoma-associated antigen 1 (MAGE-1), protein tyrosine kinase 7 (PTK7), c-type lectin domain containing 14A (CLEC14A), C-X-C chemokine receptor type 4 (CXCR4), New York esophageal squamous cell carcinoma 1 (NY-ESO-1) CD126, tumor-associated glycoprotein 72 (TAG-72), Guanylate cyclase 2C (GUCY2C), Cadherin-6 (CDH6), cadherin 17 (CDH17), claudin18.2 (CLDN18.2), glypican-3 (GPC3), CD147, CD16, glycoprotein 100 (gp100)/human leukocyte antigen-A2 (HLA-A2) complex, urokinase-type plasminogen activator receptor (uPAR), alkaline phosphatase, placental-like 2 (ALPPL2), CD47, olfactory receptor 2H1 (OR2H1), matrix metalloproteinase-2 (MMP-2), podoplanin (PDPN), GDNF family receptor alpha-4 (GFRα4), thyrotropin receptor (TSHR), L1 cell adhesion molecule (L1-CAM), or any combination thereof.

In some aspects, the chimeric antigen receptor is specific for CD19.

In some aspects, one or more nucleic acids are DNA and/or RNA. In some aspects, one or more nucleic acids are RNA.

In some aspects, the Committed CIK Precursor cells are transfected with an RNA encoding SB100X transposase and a DNA encoding a Sleeping Beauty compatible chimeric antigen receptor (CAR) transposon.

In some aspects, the transfected cells are administered with a genetically modified IL-2.

One aspect of the present disclosure is directed to genetically modified cytokine induced killer (CIK) cells obtained by the methods disclosed herein.

One aspect of the present disclosure is directed to the compositions comprising the genetically modified CIK cells disclosed herein.

In some aspects, the genetically modified CIK cells or the compositions disclosed herein are for use in the prevention or treatment of cancers, tumors, viral infections, inflammatory diseases and disorders, autoimmune diseases and disorders, or any combination thereof.

Other features and advantages of the present disclosure will be apparent from the following detailed description and examples which should not be construed as limiting. The contents of all cited references, including scientific articles, newspaper reports, GenBank entries, patents and patent applications cited throughout this application are expressly incorporated herein by reference.

### BRIEF DESCRIPTION OF THE DRAWINGS

FIG. 1 shows schematic representation of experimental flow in experiments comparing feeder free CIK process to the original process with feeder cells. Upper panel: CAR-CIK cells generation using process utilizing feeder cells and electroporation on day 0. Lower panel: feeder free process coupled with electroporation on day 2.

FIGS. 2A-2D show cell expansion parameters during the CD33.CAR-CIK cells generation process. FIGS. 2A and 2B show cell number (x10\hat{ }6) and glucose levels (mg/dl) during the day 2 feeder free CIK generation process (day 2 electroporation without feeder cells; plating the cells immediately after electroporation at 1×10⁶ cells/cm² cell density (“10\hat{ }6”) (FIG. 2A) or 2×10⁶ cells/cm² cell density (“2×10\hat{ }6”) (FIG. 2B); expanding the cells in the 6-well plates until day 7). FIG. 2C shows cell number (x10\hat{ }6) and glucose levels (mg/dl) during the Gas Permeable Rapid Expansion (G-Rex) CIK generation process where the cells were transferred to G-Rex for expansion on day 7 (10×⁶ cells/cm²) (“DAY 7”). FIG. 2D shows comparison of cell number (x10\hat{ }6) within the different culture conditions in feeder free, Day 2 electroporation CIK generation process process where cells were expanded in Flasks (“Flask”) and the G-Rex expansion process described in FIG. 2A (“10\hat{ }6”), FIG. 2B (“2×10\hat{ }6”), and FIG. 2C (“DAY 7”).

FIG. 3A shows comparison of CAR expression levels (% CD3⁺CAR⁺ cells) during the G-Rex CIK generation process (day 2 electroporation without feeder cells; plating the cells immediately after electroporation at 1×10⁶ cells/cm² cell density (“10\hat{ }6”) or 2×10⁶ cells/cm² cell density (“2×10\hat{ }6”); expanding the cells in the 6-well plates until day 7; transferring to G-Rex for expansion on day 7 (10×⁶ cells/cm²) (“DAY 7”) or expanding in Flasks (“Flask”)).

FIGS. 3B-3E show comparison of memory phenotype (T naïve (Tn) cells, T central memory cells (Tcm), effector memory cells (Tem), and terminally differentiated effector memory T cells (Temra)) during the Day 2 feeder free CIK generation process (day 2 electroporation without feeder cells; plating the cells immediately after electroporation at 1×10⁶ cells/cm² cell density (“10\hat{ }6”) (FIG. 3B) or 2×10⁶ cells/cm² cell density (“2×10\hat{ }6”) (FIG. 3C); expanding the cells in the 6-well plates until day 7; transferring to G-Rex for expansion on day 7 (10×⁶ cells/cm²) (“DAY 7”) (FIG. 3D) or expanding in Flasks (“Flask”) (FIG. 3E)).

FIGS. 3F-3I show comparison of CD4-CD8 phenotype during the day 2 feeder free CIK generation process (day 2 electroporation without feeder cells; plating the cells immediately after electroporation at 1×10⁶ cells/cm² cell density (“10\hat{ }6”) (FIG. 3F) or 2×10⁶ cells/cm² cell density (“2×10\hat{ }6”) (FIG. 3G); expanding the cells in the 6-well plates until day 7; transferring to G-Rex for expansion on day 7 (10×⁶ cells/cm²) (“DAY 7”) (FIG. 3H) or expanding in Flasks (“Flask”) (FIG. 3I)).

FIG. 3J shows CD56 expression during the day 2 feeder free CIK generation process (day 2 electroporation without feeder cells; plating the cells immediately after electroporation at 1×10⁶ cells/cm² cell density (“10\hat{ }6”) or 2×10⁶ cells/cm² cell density (“2×10\hat{ }6”); expanding the cells in the 6-well plates until day 7; transferring to G-Rex for expansion on day 7 (10×6 cells/cm²) (“DAY 7”) or expanding in Flasks (“Flask”)).

FIG. 4A shows short-term cytotoxicity assay. “No DNA”=CIK cells generated in flasks without electroporation with genetic material. “33 CAR1”=CAR-CIK-CD33 cells generated using the day 2 feeder free process, in the condition of plating the cells immediately after electroporation at 10⁶ cells/cm² cell density. “33 CAR 2”=CAR-CIK-CD33 cells generated using the day 2 feeder free process, in the condition of plating the cells immediately after electroporation at 2×10\hat{ }6 cells/cm² cell density. “33 CAR-FLASK”=CAR-CIK-CD33 cells generated using the original CIK generation process (day 0 electroporation+γ irradiated feeder cells and expanded in Flasks).

FIG. 4B shows the proliferation ability of CAR-CIK cells (% Ki-67 positive cells) evaluated after co-culture with the Cell Tracker-labeled targets THP-1 (“THP-1”) or without THP-1 (“Alone”). “No DNA”=CIK cells generated in flasks without electroporation with genetic material. “33 CAR1”=CAR-CIK-CD33 cells generated using the day 2 feeder free process, in the condition of plating the cells immediately after electroporation at 10⁶ cells/cm² cell density. “33 CAR 2”=CAR-CIK-CD33 cells generated using the day 2 feeder free process, in the condition of plating the cells immediately after electroporation at 2×10\hat{ }6 cells/cm² cell density. “33 CAR-FLASK”=CAR-CIK-CD33 cells generated using the original CIK generation process (day 0 electroporation+γ irradiated feeder cells and expanded in Flasks).

FIGS. 4C-4D show secretion profile (IL-2 production (FIG. 4C) and IFN-γ production (FIG. 4D)) in cells generated using the original CIK generation process (day 0 electroporation+γ irradiated feeder cells and expanded in Flasks) or the day 2 feeder free G-Rex process after co-culture with the Cell Tracker-labeled targets THP-1 (“THP-1”) or without THP-1 (“Alone”).

FIGS. 4E-4G show representative dot plot of the specimens' analysis by flow cytometry. FIG. 4E: Gating strategy to detect CD3+ (PerCp) CAR+ (FC-APC) cells. FIG. 4F: Gating strategy to detect CAR+ (FC-APC) cells (under the first gate) and IL-2. FIG. 4G: Gating strategy to detect CAR+ (FC-APC) cells (under the first gate) and IFN-γ.

FIGS. 5A-5B show representative dot plots of the memory phenotype analysis. FIG. 5A: CAR-CIK Donor A, expanded in flasks or in the G-REX device. FIG. 5B: CAR-CIK Donor B, expanded in flasks or in the G-REX device. Day 2/feeder free protocol was used.

FIG. 6A shows distribution of the memory phenotype in the final products produced in good manufacturing practices (GMP) for the clinical study, using the standard protocol at day 0+feeder cells and culturing the cells in Flasks.

FIG. 6B shows distribution of the memory phenotype in the G-REX CAR-CIK cultures, by using the day 2 feeder free methods and transferring the cells at day 7 in G-REX device.

FIG. 6C shows table representing the mean and standard deviation calculations on the batches of FIGS. 6A-6B.

FIG. 7A shows total number of cells recovered at the end of the cell cultures (flasks and G-REX-day 7).

FIG. 7B shows percentage of CAR expression at the end of the cell cultures (flasks and G-REX-day 7).

FIG. 7C shows percentage of CD3+CD56+ cells obtained at the end of the cell cultures (flasks and G-REX-day 7).

FIGS. 7D-7G show Day 0 (+feeder) and Day 2 (feeder free) comparison by using pT4 transposon and SB100×DNA. FIG. 7D: total number of cells recovered at the end of the cell cultures (flasks and G-REX-day 7), both using the Day 0 and the Day 2 gene transfer methods. FIG. 7E: percentage of CAR expression at the end of the cell cultures (flasks and G-REX-day 7) both using the Day 0 and the Day 2 gene transfer methods. FIG. 7F: percentage of CD3+CD56+ cells obtained at the end of the cell cultures (flasks and G-REX-day 7). FIG. 7G: percentage of CD3+CD4+ and CD3+CD8+ cells obtained at the end of the cell cultures (flasks and G-REX-day 7).

FIG. 8A shows complete immunophenotype (CD3+CD4+, CD3+CD8+, CD3+CD56+, Tn, Tcm, Tem, Temra, CAR+) of CD19.CAR-CIK cells produced in G-REX-day 7, with the SB plasmids ratio of 7.5+1.

FIG. 8B shows fold increase of CD19.CAR-CIK cells cultured both in Flasks and G-REX. Day 2/feeder free protocol was used.

FIG. 8C shows vector copy number (VCN) of CD19.CAR-CIK cells cultured in G-REX. Day 2/feeder free protocol was used.

FIG. 8D shows SB100X detection of CD19.CAR-CIK cells cultured in G-REX. Day 2/feeder free protocol was used.

FIG. 8E shows in vitro cytotoxicity of CD19.CAR-CIK cells cultured in G-REX and Flasks against the CD19+ cell line REH. Day 2/feeder free protocol used.

FIGS. 9A-9C show CD33.CAR CIK cells produced in Flasks and G-REX, by the day 2 feeder free method. FIG. 9A: total cell numbers of CD33.CAR-CIK cells cultured both in Flasks and G-REX. FIG. 9B: percentage of CD3+CAR+ CIK cells obtained at the end of the cell cultures (flasks and G-REX-day 7). FIG. 9C: Complete immunophenotype of CD33.CAR-CIK cells.

FIGS. 9D-9E show evolution of the memory phenotype (Tn, Tcm, Tem, Temra) of CD33.CAR-CIK cells produced in Flasks (FIG. 9D) and G-REX-day 7 (FIG. 9E) by using the day 2 feeder free method.

FIG. 10A shows short-term cytotoxicity assay against the CD33+ cell line THP-1 CD33.CARCIK cells generated by the Day 2 feeder free (either produced in Flasks or G-REX). “No DNA”=CIK cells generated in flasks without electroporation with genetic material.

FIG. 10B shows proliferation of CD33.CARCIK cells generated by the Day 2 feeder free (either produced in Flasks or G-REX) evaluated after co-culture with the Cell Tracker-labeled targets THP-1 (“THP-1”) or without THP-1 (“Alone”). “No DNA”=CIK cells generated in flasks without electroporation with genetic material.

FIGS. 10C-10D shows cytokine production (IL-2 (FIG. 10C) and IFN-γ (FIG. 10D)) by CD33.CARCIK cells generated by the Day 2 feeder free (either produced in Flasks or G-REX) evaluated after co-culture with the Cell Tracker-labeled targets THP-1 (“THP-1”) or without THP-1 (“Alone”). “No DNA”=CIK cells generated in flasks without electroporation with genetic material.

FIG. 11A shows absolute quantification of CD19+ DAUDI cells in mice untreated (Daudi only) and treated with of 5×10\hat{ }6 (G-REX 5) or 10×10\hat{ }6 (G-REX 10) CAR+cells/mouse during days 12, 20, 33, 40, 50, 61, 76, and 90.

FIG. 11B shows absolute quantification of CD3+ CAR-CIK cells in mice treated with of 5×10\hat{ }6 (G-REX 5) and 10×10\hat{ }6 (G-REX 10) CAR+cells/mouse during days 12, 20, 33, 40, 50, 61, 76, and 90.

FIG. 11C shows survival curve analysis in mice untreated (Daudi only) and treated with of 5×10\hat{ }6 (G-REX 5) or 10×10\hat{ }6 (G-REX 10) CAR+ cells/mouse.

FIGS. 11D-11F show representative dot plot flow cytometric analysis of in vivo specimens. FIG. 11D: Gating strategy to select human CD45+ cells out of mouse CD45+ cells. FIG. 11E: Dot plot of CD19+CD10+ DAUDI cells in the bone marrow. FIG. 11F: Dot plot to detect CD3+ CAR-CIK cells and CD19+ DAUDI cells.

FIG. 12 shows composition of CAR-CIK-CD19 cultures throughout the production process.

FIGS. 13A-13M show representative gating strategy to identify CD25, CD69, CD137 and HLA-DR positive cells on day 0. Single cells are identified by gating on FSC-H and FSC-A (FIG. 13A). Viable cells are shown in the Aquadye-negative gate (FIG. 13B). Viable CD3+ cells for day 0 PBMCs (FIG. 13C), gated CD3+CD4+ cells (FIG. 13D) and CD8+ cells (FIG. 13E) are shown. The activation markers CD25, CD69, CD137, and HLA-DR area reported for both CD8+ cells (FIG. 13F, FIG. 13G, FIG. 13H, FIG. 13I, respectively) and CD4+ cells (FIG. 13J, FIG. 13K, FIG. 13L, FIG. 13M, respectively) within each respective gate.

FIGS. 14A-14M show representative gating strategy to identify CD25, CD69, CD137 and HLA-DR positive cells on day 2. Single cells are identified by gating on FSC-H and FSC-A (FIG. 14A). Viable cells are shown in the Aquadye-negative gate (FIG. 14B). Day 2 cells are gated on all viable cells (FIG. 14C) then subgated to identify viable CD4+ cells (FIG. 14D) and CD8+ cells (FIG. 21E) are shown. The activation markers CD25, CD69, CD137, and HLA-DR area reported for both CD8+ cells (FIG. 14F, FIG. 14G, FIG. 14H, FIG. 14I, respectively) and CD4+ cells (FIG. 14J, FIG. 14K, FIG. 14L, FIG. 14M) within each respective gate.

FIGS. 15A-15B show activation marker upregulated on day 2 cells. The percentage of CD8+ cells (FIG. 15A) and CD4+ cells (FIG. 15B) on day 0 CD3+ cells (□ bars) and Day 2 CIK cells (▪bars) are shown for each of the activation markers CD25, CD69, CD137, and HLA-DR.

FIG. 16 shows time course of CAR expression during the 21 day culture period after electroporation with pT4-CD19-CAR plasmid DNA and pCMV-SB100X plasmid DNA. The percentage of CAR positive, CD3+ cells is shown for days 7, 14 and 21 of the cell culture period. Day 2 activated cells were electroporated with plasmid DNA encoding the CAR gene and SB100X transposase plasmid DNA in different electroporation buffers BioRad, CoStorSol Lonza and OptiMEM. Mock cells were electroporated in CoStorSol in the absence of any nucleic acids.

FIGS. 17A-17J show CAR positivity post electroporation with pT4-CD19-CAR plasmid DNA and pCMV-SB100X plasmid DNA. Day 2 activated cells were electroporated with pT4-CD19CAR plasmid DNA and pCMV-SB100X plasmid DNA in the four electroporation buffers tested, BioRad, CoStorSol, Lonza and OptiMEM. Representative pseudo-color dot plots showing the percentage of CAR positive cells (CAR+ positivity) are shown for days 14 (BioRad (FIG. 17B), CoStorSol (FIG. 17C), Lonza (FIG. 17D) and OptiMEM (FIG. 17E)) and 21 (BioRad (FIG. 17G), CoStorSol (FIG. 17H), Lonza (FIG. 17I) and OptiMEM (FIG. 17J)) of the cell culture periods. Mock refers to day 2 activated cells shown for day 14 (FIG. 17A) and day 21 (FIG. 17F) that were electroporated in the absence of nucleic acids. The plots shown represent the viable CD3+ cell population. Numbers in the black gated regions represent the percentage of CAR positive cells within the CD3+ cell population.

FIG. 18 show time course of CAR expression during the 21-day culture period after electroporation with pT4-CD19CAR plasmid DNA and Capped-SB100X RNA. The percentage of CAR positive, CD3+ cells is shown for days 7, 14 and 21 of the culture period. Day 2 activated cells were electroporated with plasmid DNA encoding the CAR gene and SB100X transposase RNA in different electroporation buffers BioRad, CoStorSol Lonza and OptiMEM. Mock cells were electroporated in CoStorSol in the absence of any nucleic acids.

FIGS. 19A-19J show CAR positivity post electroporation with pT4-CD19CAR plasmid DNA and Capped-SB100X RNA. Day 2 activated cells were electroporated with pT4-CD19CAR plasmid DNA and SB 100X transposase RNA in the four electroporation buffers tested, BioRad, CoStorSol, Lonza and OptiMEM. Representative pseudo-color dot plots showing the percentage of CAR positive cells (CAR+ positivity) are shown for days 14 (BioRad (FIG. 19B), CoStorSol (FIG. 19C), Lonza (FIG. 19D) and OptiMEM (FIG. 19E)) and 21 (BioRad (FIG. 19G), CoStorSol (FIG. 19H), Lonza (FIG. 19I) and OptiMEM (FIG. 19J)) of the cell culture periods. Mock refers to day 2 activated cells shown for day 14 (FIG. 19A) and day 21 (FIG. 19F) that were electroporated in the absence of nucleic acids. The plots shown represent the viable CD3+ cell population. Numbers in the black-gated regions represent the percentage of CAR positive cells within the CD3+ cell population.

FIGS. 20A-20C show T cell enrichment, T cell activation, and electroporation efficiency and CAR expression in lots initiated from cryopreserved PBMCs (frozen) versus fresh PBMCs (fresh). FIG. 20A shows the % of T cells in the culture on day 2 (% T cells (d2)). FIG. 20B shows T cell activation by evaluating the expression of CD25 activation marker on T cells (% CD25+ T cells). FIG. 20C shows % CD3+CAR+ expression on day 7. * p-value≤0.05, ** p-value≤0.01 by unpaired t test with Welch's correction; n=10; ns=no significant difference.

FIGS. 21A-21B show the effect of PBMC cryopreservation (frozen v. fresh PBMCs) on the fold expansion and memory phenotype of CARCIK-1918 cells. FIG. 21A shows a fold expansion calculated from day 2 (electroporation) to day 17 (D2-D17). FIG. 21B shows a memory phenotype (central memory and effector memory) of CD3+CAR+ cells at day 17. T-Unpaired t test with Welch's correction; n=6; ns=no significant difference.

FIGS. 22A-22B show in vitro function of CARCIK-1918 cells manufactured from fresh or frozen PBMCs. CARCIK-1918 cells were characterized by in vitro stimulation with CD19+ REH tumor cells to assess tumor killing activity (% cytotoxicity) (FIG. 22A) and IL-18 secretion (FIG. 22B). T-Unpaired t test with Welch's correction; n=2-5; ns=no significant difference.

FIG. 23 shows the representative plots of the expression of CD25 activation marker in viable T cells manufactured from fresh or frozen PBMCs.

FIG. 24 shows a diagram of bicistronic CAR transgenes linked to IL-18 gene. Plasmids A and B contain a 3^{rd} generation CAR transgene encoding the FMC63 scFv domain linked to the human IL-18 gene. Plasmids C and D contain a 2^{nd} generation CAR transgene encoding the SJ25C1 scFv domain linked to the human IL-18 gene. Plasmids B and D contain a CD28 signaling domain with the amino acid substitution YMNM→YSNV. All four plasmids contain a P2A cleavage site to allow the secretion of IL-18.

FIG. 25 shows a schematic representation of study design in NSG/Raji survival model.

FIG. 26 shows bioluminescence imaging (BLI) of mice treated with CARCIK-1918 cells. Mice were either left untreated or treated with Arm A (3^{rd} gen pT4-CD19CAR-IL18), Arm B (3^{rd} gen pT4-CD19CARYSNV-IL18), Arm C (2^{nd} gen pT4-1928z-IL18), and Arm D (2^{nd} gen pT4-1928YSNVz-18) CARCIK-1918 cells. BLI measurements were taken at weekly intervals, starting at week 1 and at weeks 2, 3, 4, and 5.

FIG. 27 shows an average radiance of tumor burden in mice treated with CARCIK-1918 cells. Quantification of BLI measured as average radiance (p/s/cm3/sr) for Region of Interest (ROI) for untreated mice and mice treated with Arm A (3^{rd} gen pT4-CD19CAR-IL18), Arm B (3^{rd} gen pT4-CD19CARYSNV-IL18), Arm C (2^{nd} gen pT4-1928z-IL18), and Arm D (2^{nd} gen pT4-1928YSNVz-18) CARCIK-1918 cells.

FIG. 28 shows the analysis of animal survival (probability of survival) using Kaplan-Meier curves for untreated mice and mice treated with Arm A (3^{rd} gen pT4-CD19CAR-IL18), Arm B (3^{rd} gen pT4-CD19CARYSNV-IL18), Arm C (2^{nd} gen pT4-1928z-IL18), and Arm D (2^{nd} gen pT4-1928YSNVz-18) CARCIK-1918 cells.

FIG. 29 shows measurement of animal body weight (grams) recorded at least twice weekly throughout the study until mice succumbed to disease or reached the humane endpoint (day (D) 0, D3, D7, D10, D14, D17, D22, D25, D28, D35, D38, D42, D45, D49, D52, D57, D59, D63, D66, D70, D73, D77, D84, and D86) for untreated mice and mice treated with Arm A (3^{rd} gen pT4-CD19CAR-IL18), Arm B (3^{rd} gen pT4-CD19CARYSNV-IL18), Arm C (2^{nd} gen pT4-1928z-IL18), and Arm D (2^{nd} gen pT4-1928YSNVz-18) CARCIK-1918 cells. Data is presented as mean±/−std for each group.

FIGS. 30A-30C show an exploratory analysis of CARCIK-1918 cell persistence in vivo. FIG. 30A shows numbers of human CD4+CD3+ cells and CD8+CD3+ cells present in the peripheral blood for sacrificed mice on day 22 either untreated or treated with Arm A (3rd gen pT4-CD19CAR-IL18) and Arm B CARCIK-1918 cells (3rd gen pT4-CD19CARYSNV-IL18). FIG. 30B shows numbers of human CD4+CD3+ cells and CD8+CD3+ cells present in the bone marrow for sacrificed animals on day 22 either untreated or treated with Arm A (3rd gen pT4-CD19CAR-IL18) and Arm B (3rd gen pT4-CD19CARYSNV-IL18) CARCIK-1918 cells. FIG. 30C shows numbers of human CD4+CD3+ cells and CD8+CD3+ cells present in the peripheral blood for one mouse sacrificed on day 46 treated with Arm D (2nd gen pT4-1928YSNVz-18) CARCIK-1918 cells. The number of CD4 and CD8 CD3 cells is reported from the viable cell gate as number of cells/mL of blood.

FIGS. 31A-31C show cytokine measurement in peripheral blood of mice treated with CARCIK-1918 cells. Peripheral blood was collected from untreated mice and mice treated with Arm A (3^{rd} gen pT4-CD19CAR-IL18), Arm B (3^{rd} gen pT4-CD19CARYSNV-IL18), Arm C (2^{nd} gen pT4-1928z-IL18), and Arm D (2^{nd} gen pT4-1928YSNVz-18) CARCIK-1918 cells on day 8. Cytokine levels for GM-CSF (FIG. 31A), IFN-γ (FIG. 31B) and TNF-α (FIG. 31C) in the peripheral blood were measured by cytokine bead array and reported as pg/mL of blood.

FIG. 32 shows bioluminescence imaging of mice treated with CARCIK-1918 cells. Mice were either left untreated or treated with Arm A (3^{rd} gen pT4-CD19CAR-IL18), Arm B (3^{rd} gen pT4-CD19CARYSNV-IL18), Arm C (2^{nd} gen pT4-1928z-IL18), and Arm D (2^{nd} gen pT4-1928YSNVz-18) CARCIK-1918 cells. BLI measurements were taken at weekly intervals, starting at week 2, and at weeks 3, 4 and 5.

FIG. 33 shows an average radiance of tumor burden in mice treated with CARCIK-1918. Quantification of BLI measured as average radiance (p/s/cm3/sr) for ROI for untreated mice and mice treated with Arm A (3^{rd} gen pT4-CD19CAR-IL18), Arm B (3^{rd} gen pT4-CD19CARYSNV-IL18), Arm C (2^{nd} gen pT4-1928z-IL18), and Arm D (2^{nd} gen pT4-1928YSNVz-18) CARCIK-1918 cells.

FIG. 34 shows an average radiance of tumor burden in individual mice treated with CARCIK-1918 cells, day 14. Quantification of BLI measured as average radiance (p/s/cm3/sr) for ROI for untreated mice and mice treated with Arm A (3^{rd} gen pT4-CD19CAR-IL18), Arm B (3^{rd} gen pT4-CD19CARYSNV-IL18), Arm C (2^{nd} gen pT4-1928z-IL18), and Arm D (2^{nd} gen pT4-1928YSNVz-18) CARCIK-1918 cells. Each symbol represents an individual animal. Mean and std are shown.

FIG. 35 shows animal survival (probability of survival) using Kaplan-Meier curves for untreated mice and mice treated with Arm A (3^{rd} gen pT4-CD19CAR-IL18), Arm B (3rd gen pT4-CD19CARYSNV-IL18), Arm C (2nd gen pT4-1928z-IL18), and Arm D (2nd gen pT4-1928YSNVz-18) CARCIK-1918 cells.

FIG. 36 shows measurement of animal body weight (grams) recorded at least twice weekly throughout the study until mice succumbed to disease or reached the humane endpoint (day (D) 2, D5, D8, D12, D15, D19, D22, D26, D29, D33, D40, D44, D47, and D51) for untreated mice and mice treated with Arm A (3^{rd} gen pT4-CD19CAR-IL18), Arm B (3^{rd} gen pT4-CD19CARYSNV-IL18), Arm C (2^{nd} gen pT4-1928z-IL18), and Arm D (2^{nd} gen pT4-1928YSNVz-18) CARCIK-1918 cells. Data is presented as mean±/−std for each group.

FIGS. 37A-37B show an exploratory analysis of tumor cell engraftment in mice treated with CARCIK-1918 cells. Peripheral blood was collected on day 8 for 3 mice from either untreated or treaded with Arm A (3^{rd} gen pT4-CD19CAR-IL18), Arm B (3^{rd} gen pT4-CD19CARYSNV-IL18), Arm C (2^{nd} gen pT4-1928z-IL18), and Arm D (2^{nd} gen pT4-1928YSNVz-18) CARCIK-1918 cells and the numbers (cells/ml) (FIG. 37A) and frequency (% of the viable cells) (FIG. 37B) of human CD19+ Raji tumor cells present in the peripheral blood was determined by flow cytometry. Data is reported as cells/mL of human CD19 positive cells in the viable cell gate.

FIGS. 38A-38B show an exploratory analysis of CARCIK-1918 cell persistence. Peripheral blood was collected on day 8 for 3 mice from either untreated or treaded with Arm A (3^{rd} gen pT4-CD19CAR-IL18), Arm B (3^{rd} gen pT4-CD19CARYSNV-IL18), Arm C (2^{nd} gen pT4-1928z-IL18), and Arm D (2^{nd} gen pT4-1928YSNVz-18) CARCIK-1918 cells and the numbers (cells/ml) (FIG. 37A) and frequency (% of the viable cells) (FIG. 37B) of human CD3+ cells present in the peripheral blood was determined by flow cytometry. Data is reported as cells/mL of human CD3 positive cells in the viable cell gate.

FIGS. 39A-39B show an exploratory analysis of CARCIK-1918 cell persistence in vivo in mice treated with CARCIK-1918 cells. FIG. 39A shows numbers of human CD4+CD3+ cells and CD8+CD3+ cells present in the peripheral blood collected on day 8 from animals untreated or treated with Arm A (3^{rd} gen pT4-CD19CAR-IL18), Arm B (3^{rd} gen pT4-CD19CARYSNV-IL18), Arm C (2^{nd} gen pT4-1928z-IL18), and Arm D (2^{nd} gen pT4-1928YSNVz-18) CARCIK-1918 cells. FIG. 39B shows numbers of human CD4+CD3+ cells and CD8+CD3+ cells present in the peripheral blood for one mouse sacrificed on day 40 treated with Arm D (2^{nd} gen pT4-1928YSNVz-18) CARCIK-1918 cells.

FIGS. 40A-40C show cytokine measurement in peripheral blood of mice treated with CARCIK-1918 cells. Peripheral blood was collected from untreated mice and mice treated with Arm A (3^{rd} gen pT4-CD19CAR-IL18), Arm B (3^{rd} gen pT4-CD19CARYSNV-IL18), Arm C (2^{nd} gen pT4-1928z-IL18), and Arm D (2^{nd} gen pT4-1928YSNVz-18) CARCIK-1918 cells. Cytokine levels for GM-CFS (FIG. 40A), IFN-γ (FIG. 40B) and TNF-α (FIG. 40C) in the peripheral blood were measured by cytokine bead array and reported as pg/mL of blood.

FIG. 41 shows a schematic representation of study design in NSG/Daudi survival model.

FIG. 42 shows the mean concentration of hCD45+/hCD19+ cells/mL of blood for each experimental group (DAUDI only, CARCIK-1918 5×10\hat{ }6, CARCIK-1918 10×10\hat{ }6) was plotted versus sampling day (days 10, 20, 30, 40, 50, 60, 70, 80, and 90) to monitor cell expansion over the course of the study.

FIG. 43 shows results of flow cytometry analysis of peripheral blood collected across the course of the study. CARCIK-1918 cells were identified by staining for the human CD45 and human CD3 cell surface markers. The mean concentration (cells/mL) in the blood was plotted versus the sampling day for each experimental group (CARCIK-1918 5×10\hat{ }6, CARCIK-1918 10×10\hat{ }6).

FIG. 44 shows mean body weight (g) of the mice in each experimental group (DAUDI only, CARCIK-1918 5×10\hat{ }6, CARCIK-1918 10×10\hat{ }6) monitored at least twice weekly until the animals succumbed to disease or reached the humane endpoint (days 0, 5, 9, 14, 20, 23, 26, 33, 37, 43, 49, 54, 58, 62, 65, 67, 72, 76, 83, and 90).

FIG. 45 shows an estimated animal survival (probability of survival) curves in each experimental group (DAUDI only, IL18 CAR 5×10\hat{ }6, IL18 CAR 10×10\hat{ }6) based on the Kaplan-Meier method.

FIGS. 46A-46D show percentage (%) of hCD45+/hCD19+ (filled circles, Daudi) and hCD45+/hCD3+ (open circles, hCD3+) cells in the bone marrow (BM) (FIG. 46A), peripheral blood (PB) (FIG. 46B), spleen (FIG. 46C), and kidney (FIG. 46D) at sacrifice for each experimental group as indicated on the X axes (DAUDI only, CARCIK-1918 5×10\hat{ }6, CARCIK-1918 10×10\hat{ }6).

### DETAILED DESCRIPTION OF THE DISCLOSURE

Disclosed herein are methods of generating genetically modified cytokine induced killer (CIK) cells, comprising the sequential steps of: (a) culturing a population of mononuclear cells in culture medium comprising at least one differentiating agent to induce differentiation of the mononuclear cells in a cell culture into Committed CIK Precursor cells; (b) adding at least one stimulating agent and at least one expanding agent to the cell culture; and (c) expanding the cells from the cell culture to obtain a cell population comprising Committed CIK Precursor cells, transfecting the cell population comprising the Committed CIK Precursor cells with one or more nucleic acids to produce genetically modified Committed CIK Precursor cells, and expanding the genetically modified Committed CIK Precursor cells in culture medium to produce the genetically modified CIK cells; or (c) expanding the cells from the cell culture to obtain a cell population comprising Committed CIK Precursor cells and transfecting the cell population comprising the Committed CIK Precursor cells with one or more nucleic acids; (d) formulating transfected cells comprising the Committed CIK Precursor cells for administration to a subject in need thereof; and (e) administering the transfected cells to the subject, wherein transfected Committed CIK Precursor cells are expanded to produce the genetically modified CIK cells in the subject; wherein steps (a), (b) and (c) are performed in the absence of non-irradiated or irradiated feeder cells. In some aspects, the mononuclear cells are PBMCs. In some aspects, the PBMCs were previously cryopreserved and thawed before culturing in step (a).

### I. DEFINITIONS

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

As used herein, the terms “comprises”, “comprising”, “includes”, “including”, “having,” and their conjugates mean “including but not limited to.”

As used herein, the term “consisting of” means “including and limited to.”

As used herein, the term “consisting essentially of” means the specified material of a composition, or the specified steps of a method, and those additional materials or steps that do not materially affect the basic characteristics of the material or method.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower). The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

As used herein, the term “approximately,” as applied to one or more values of interest, refers to a value that is similar to a stated reference value. In some aspects, the term “approximately,” like the term, “about,” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range, or integer range is to be understood to include the value of any integer within the recited range and, when appropriate, fractions thereof (such as one tenth and one hundredth of an integer), unless otherwise indicated.

The terms “antibody” and “antibodies” are terms of art and can be used interchangeably herein and refer to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity.

An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain an antigen recognition site of an intact antibody (e.g., complementarity determining regions (CDRs) sufficient to bind antigen). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

The term “antigen” is well understood in the art and includes substances which are immunogenic, i.e., immunogen. It will be appreciated that the use of any antigen is envisioned for use in the present disclosure and thus includes, but is not limited to a self-antigen (whether normal or disease-related), an infectious antigen (e.g., a microbial antigen, viral antigen, etc.), or some other foreign antigen (e.g., a food component, pollen, etc.). The term “antigen” or alternatively, “immunogen” applies to collections of more than one immunogen, so that immune responses to multiple immunogens can be modulated simultaneously. Moreover, the term includes any of a variety of different formulations of immunogen or antigen. Furthermore, the antigen can be from a cancer cell (e.g., a renal cancer cell, a multiple myeloma cell, and a melanoma cell) or a pathogen (e.g., HIV and HCV). The antigen can be delivered to the antigen presenting cell (APC) in the form of RNA isolated or derived from a cancer cell or a pathogen. “Derived from” includes, but is not limited recombinant variants of naturally occurring sequences, including fusions to unrelated or related sequences. Methods for RT-PCR of RNA extracted from any cell (e.g., a cancer cell or pathogen cell), and in vitro transcription are disclosed, for example in PCT/US05/053271.

A “native” or “natural” or “wild-type” antigen is a polypeptide, protein or a fragment which contains an epitope, which has been isolated from a natural biological source, and which can specifically bind to an antigen receptor, when presented as an HLA/peptide complex, in particular a T cell antigen receptor (TCR), in a subject.

As used herein, an “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). Epitopes formed from contiguous amino acids are typically, but not always, retained on exposure to denaturing solvents, whereas epitopes formed by tertiary folding are typically lost on treatment with denaturing solvents. An epitope typically includes at least 1, 2, 3, 4, 5, 6, 7, 8, 9, 10, 11, 12, 13, 14, 15, or 20 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from (e.g., CD16) are tested for reactivity with a given antibody (e.g., anti-human CD16 antibody). Methods of determining spatial conformation of epitopes include techniques in the art and those described herein, for example, x-ray crystallography, 2-dimensional nuclear magnetic resonance and HDX-MS (see, e.g., Epitope Mapping Protocols in Methods in Molecular Biology, Vol. 66, G. E. Morris, Ed. (1996)).

In certain aspects, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization can be accomplished using any of the known methods in the art (e.g., Giege R et al., (1994) Acta Crystallogr D Biol Crystallogr 50(Pt 4): 339-350; McPherson A (1990) Eur J Biochem 189:1-23; Chayen N E (1997) Structure 5:1269-1274; McPherson A (1976) J Biol Chem 251:6300-6303). Antibody: antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323). Mutagenesis mapping studies can be accomplished using any method known to one of skill in the art. See, e.g., Champe M et al., (1995) J Biol Chem 270:1388-1394 and Cunningham B C & Wells J A (1989) Science 244:1081-1085 for a description of mutagenesis techniques, including alanine scanning mutagenesis techniques.

The term “epitope mapping” refers to the process of identification of the molecular determinants for antibody-antigen recognition.

The terms “major histocompatibility complex” or “MHC” refers to a complex of genes encoding cell-surface molecules that are required for antigen presentation to T cells and for rapid graft rejection. In humans, the MHC is also known as the “human leukocyte antigen” or “HLA” complex. The proteins encoded by the MHC are known as “MHC molecules” and are classified into Class I and Class II MHC molecules. Class I MHC molecules include membrane heterodimeric proteins made up of an α chain encoded in the MHC noncovalently linked with the β₂-microglobulin. Class I MHC molecules are expressed by nearly all nucleated cells and have been shown to function in antigen presentation to CD8⁺ T cells. Class I molecules include HLA-A, B, and C in humans. Class II MHC molecules also include membrane heterodimeric proteins consisting of noncovalently associated α and β chains. Class II MHC molecules are known to function in CD4⁺ T cells and, in humans, include HLA-DP, -DQ, and -DR.

The term “culturing” as used herein refers to the controlled growth of cells ex vivo and/or in vitro. As used herein, “culturing” includes the growth of cells, e.g., one or more Committed CIK Precursor cell disclosed herein, during cell expansion, or cell engineering (e.g., transfecting the cell population comprising the Committed CIK Precursor cells with one or more nucleic acids (e.g., co-electroporation with non-viral DNA construct encoding CAR (e.g., pT4-CD19-CAR) and mRNA encoding transposase (e.g., mRNA encoding SB100X transposase)). In some aspects, the cultured cells are obtained from a subject, e.g., a human subject/patient (e.g., mononuclear cells are isolated from a human leukocyte antigen (HLA) matched donor). In some aspects, the cultured cells comprise cells obtained from a human subject. In some aspects, the cultured cells comprise one or more engineered cell disclosed herein (e.g., transfected cells comprising the Committed CIK Precursor cells). In some aspects, the cultured cells comprise PBMCs obtained from a human subject/patient.

The term “expand,” “expanding,” or “expansion,” as used herein in reference to cell culture refers to the process of stimulating or activating the cells and culturing the cells. The expansion process can lead to an increase in the proportion or the total number of desired cells, e.g., an increase in the proportion or total number of cells in a population of cultured cells, after the cells are stimulated or activated and cultured. Expansion does not require that all cell types in a population of cultured cells are increased in number. Rather, in some aspects, only a subset of cells in a population of cultured cells are increased in number during expansion, while the number of other cell types may not change or may decrease.

As used herein, the term “yield” refers to the total number of cells following a culture method or a portion thereof. In some aspects, the term “yield” refers to a particular population of cells, e.g., Committed CIK Precursor cells in a population cultured cells or genetically modified Committed CIK Precursor cells in a population cultured cells. The yield can be determined using any methods, including, but not limited to, estimating the yield based on a representative sample.

As used herein, the term “immune cell” refers to a cell of the immune system. In some aspects, the immune cell is selected from a T lymphocyte (“T cell”), B lymphocyte (“B cell”), natural killer (NK) cell, natural killer T lymphocytes (NKT cells), macrophage, eosinophil, mast cell, dendritic cell or neutrophil.

As used herein, a “population” of cells refers to a collection of more than one cell, e.g., a plurality of cells. In some aspects, the population of cells comprises more than one immune cell, e.g., a plurality of immune cells. In some aspects, the population of cells is comprises a heterogeneous mixture of cells, comprising multiple types of cells, e.g., a heterogeneous mixture of immune cells and non-immune cells. In some aspects, the population of cells comprises a population of mononuclear cells. In some aspects, the population of cells comprises a population comprising Committed CIK Precursor cells. In some aspects, the population of cells comprises a population comprising genetically modified CIK cells.

As used herein, the terms “T cell” and “T lymphocyte” are interchangeable and refer to any lymphocytes produced or processed by the thymus gland. Non-limiting classes of T cells include effector T cells and T helper (Th) cells (such as CD4⁺ or CD8⁺ T cells). In some aspects, the T cell is a Th1 cell. In some aspects, the T cell is a Th2 cell. In some aspects, the T cell is a Tc17 cell. In some aspects, the T cell is a Th17 cell. In some aspects, the T cell is a T_{reg} cell. In some aspects, the T cell is a tumor-infiltrating cell (TIL).

As used herein, the term “memory” T cells refers to T cells that have previously encountered and responded to their cognate antigen (e.g., in vivo, in vitro, or ex vivo) or which have been stimulated, e.g., with an anti-CD3 antibody (e.g., in vitro or ex vivo). Immune cells having a “memory-like” phenotype upon secondary exposure, such memory T cells can reproduce to mount a faster and strong immune response than during the primary exposure. In some aspects, memory T cells comprise central memory T cells (T_{CM} cells), effector memory T cells (T_{EM} cells), tissue resident memory T cells (T_{RM} cells), stem cell-like memory T cells (T_{SCM} cells), or any combination thereof.

As used herein, the term “stem cell-like memory T cells,” “T memory stem cells,” or “T_{SCM} cells” refers to memory T cells that express CD95, CD45RA, CCR7, and CD62L and are endowed with the stem cell-like ability to self-renew and the multipotent capacity to reconstitute the entire spectrum of memory and effector T cell subsets.

As used herein, the term “central memory T cells” or “T_{CM} cells” refers to memory T cells that express CD45RO, CCR7, and CD62L. Central memory T cells are generally found within the lymph nodes and in peripheral circulation.

As used herein, the term “effector memory T cells” or “T_{EM} cells” refers to memory T cells that express CD45RO but lack expression of CCR7 and CD62L. Because effector memory T cells lack lymph node-homing receptors (e.g., CCR7 and CD62L), these cells are typically found in peripheral circulation and in non-lymphoid tissues.

As used herein, the term terminally differentiated effector memory T cells,” “TEMRA” or “Temra” refers to CD8 T cells which constitute a preformed effector population with an enhanced expression of effector molecules that can be efficiently activated using TCR stimulation alone or in combination with common-gamma chain cytokines. (Tilly, G et al., TEMRA CD8 T Cells Are Highly Cytopathic Cells That Escape From Costimulatory Based-Therapy.: Abstract #B1159. Transplantation, 98:318-319 (2014).

As used herein, the term “tissue resident memory T cells” or “T_{RM} cells” refers to memory T cells that do not circulate and remain resident in peripheral tissues, such as skin, lung, and gastrointestinal tract. In some aspects, tissue resident memory T cells are also effector memory T cells.

As used herein, the term “naïve T cells,” “Tₙ cells,” or “T_{N} cells” refers to T cells that express CD45RA, CCR7, and CD62L, but which do not express CD95. T_{N} cells represent the most undifferentiated cell in the T cell lineage. The interaction between a T_{N} cell and an antigen presenting cell (APC) induces differentiation of the T_{N} cell towards an activated TEFF cell and an immune response.

As used herein, the term “cytokine” refers to refers to small, secreted proteins released by cells that have a specific effect on the interactions and communications between cells. In some aspects, cytokines can possess one or more of the following properties: ability to mediate and/or regulate immune defense functions by acting as messengers between the various immune cells; functioning over short distances with a brief half-life; produced by a variety of cells types; ability to act on diverse cell targets within the immune system and/or on organs; ability to stimulate and/or inhibit growth; and/or directly or indirectly causeing a cytokine cascade. In some aspects, cytokines can include interleukins, interferons, colony stimulating factors and tumour necrosis factor. Non-limiting examples of cytokines which can be used alone or in combination in the practice of the present disclosure include, tnterleukin-1 alpha (IL-1 alpha or IL-1a), interleukin-1 beta (IL-1 beta or IL-1b), interleukin-2 (IL-2), stem cell factor (SCF), interleukin-3 (IL-3), interleukin-4 (IL-4), interleukin-5 (IL-5), interleukin-6 (IL-6), interleukin-7 (IL-7), interleukin-10 (IL-10), interleukin-11 (IL-11), interleukin-12 (IL-12), interleukin-13 (IL-13), interleukin-15 (IL-15), interleukin-17 (IL-17), interleukin-18 (IL-18), interleukin-21 (IL-21), interleukin-22 (IL-22), interleukin-23 (IL-23), interleukin-27 (IL-27), interleukin-33 (IL-33), granulocyte-colony stimulating factor (G-CSF), granulocyte macrophage-colony stimulating factor (GM-CSF), interleukin-1 beta (IL-1β), interferon-α (IFN-α), interferon-γ (IFNγ), tumor necrosis factor-α (TNFα), prostaglandin E₂ (PGE₂), MIP-11, leukemia inhibitory factor (LIF), c-kit ligand, thrombopoietin (TPO), and flt3 ligand. Cytokines are commercially available from several vendors such as, for example, Genzyme (Framingham, Mass.), Genentech (South San Francisco, Calif.), Amgen (Thousand Oaks, Calif.), R&D Systems (Minneapolis, Minn.) and Immunex (Seattle, Wash.). It is intended, although not always explicitly stated, that molecules having similar biological activity as wild-type or purified cytokines (e.g., recombinantly produced or muteins thereof) are intended to be used within the spirit and scope of the disclosure.

As used herein, the term “cytokine receptor” refers to the cell-surface glycoproteins that bind specifically to cytokines and transduce their signals. These receptors enable cells to communicate with the extracellular environment by responding to signals generated in the vicinity or in other parts of the organism. Non-limiting examples of cytokine receptors which can be used alone or in combination in the practice of the present disclosure include, type I cytokine receptors (e.g., type 1 interleukin receptors (e.g., IL-15 receptor), erythropoietin receptor, GM-CSF receptor, G-CSF receptor, growth hormone receptor, prolactin receptor, oncostatin M receptor, eukemia inhibitory factor receptor); type II cytokine receptors (e.g., type II interleukin receptors, interferon-alpha/beta receptor, interferon-gamma receptor); members of the immunoglobulin superfamily (e.g., interleukin-1 receptor, CSF1, C-kit receptor, interleukin-18 receptor); tumor necrosis factor receptor family (e.g., CD27, CD30, CD40, CD120, lymphotoxin beta receptor); TGF-beta receptor family (TGF beta receptor 1, TGF beta receptor 2); and chemokine receptors. In some aspects, the cytokine receptor is interleukin-2 receptor alpha chain (also called CD25). In some aspects, the cytokine receptor is interleukin-2 receptor subunit beta (also called CD122; IL15RB; P70-75). In some aspects, the cytokine receptor is interleukin-2 receptor subunit gamma (also called CD122; IL-2RG).

As used herein, the term “chemokine” or “chemotactic cytokine” refers to a family of small cytokines or signaling proteins secreted by cells that induce directional movement of leukocytes, as well as other cell types, including endothelial and epithelial cells. In addition to playing a major role in the activation of host immune responses, chemokines are important for biological processes, including morphogenesis and wound healing, as well as in the pathogenesis of diseases like cancers. Chemokines have been classified into four main subfamilies: CXC, CC, CX3C and C. All of these proteins exert their biological effects by interacting with G protein-linked transmembrane receptors called chemokine receptors, that are selectively found on the surfaces of their target cells. Non-limiting examples of cytokine receptors which can be used alone or in combination in the practice of the present disclosure include, CCL1, CCL2, CCL3, CCL4, CCL5, CCL6, CCL7, CCL8, CCL9/CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, and XCL2, CX3CL1.

As used herein, the term “chemokine receptor” refers to cytokine receptors found on the surface of certain cells that interact with a type of cytokine called a chemokine. Chemokine receptors are divided into different families, CXC chemokine receptors, CC chemokine receptors, CX3C chemokine receptors and XC chemokine receptors that correspond to the 4 distinct subfamilies of chemokines they bind. Four families of chemokine receptors differ in spacing of cysteine residues near N-terminal of the receptor. Non-limiting examples of chemokine receptors which can be used alone or in combination in the practice of the present disclosure include, CXCR1, CXCR2, CXCR3, CXCR4, CXCR5, CXCR6, CCR1, CCR2, CCR3, CCR4, CCR5, CCR6, CCR7, CCR8, CCR9, CCR10, CCR11, XCR1, and CX3CR1.

As used herein, the term “cell adhesion molecule,” or “CAM,” refers to a subset of cell surface proteins that are involved in the binding of cells with other cells or with the extracellular matrix (ECM), in a process called cell adhesion. In essence, CAMs help cells stick to each other and to their surroundings. CAMs are crucial components in maintaining tissue structure and function. In fully developed animals, these molecules play an integral role in generating force and movement and consequently ensuring that organs are able to execute their functions normally. In addition to serving as “molecular glue,” CAMs play important roles in the cellular mechanisms of growth, contact inhibition, and apoptosis. Aberrant expression of CAMs can result in a wide range of pathologies, ranging from frostbite to cancer. Non-limiting examples of the cell adhesion molecules which can be used alone or in combination in the practice of the present disclosure include, (i) the immunoglobulin super family of cell adhesion molecules (IgCAMs) (e.g., neural cell adhesion molecule (N-CAM), intercellular adhesion molecule (ICAM)-1, ICAM-2, ICAM-3, ICAM-4, ICAM-5, vascular cell adhesion molecule-1 (VCAM-1), platelet endothelial cell adhesion molecule (PE-CAM-1), L1 family (e.g., L1-CAM, neuronal cell adhesion molecule (NRCAM), neurofascin (NFASC), neural cell adhesion molecule L1-like protein also known as close homolog of L1 (CHL1)), Nectin (e.g., poliovirus receptor-related 1 (PVRL1), PVRL2, PVRL3, cell adhesion molecule 1 (CADM1), CADM3, CD155)); (ii) Integrins (e.g., LFA-1 (CD11a+CD18), integrin alphaXbeta2 (CD11c+CD18), macrophage-1 antigen (CD11b+CD18), VLA-4 (CD49d+CD29), glycoprotein IIb/IIIa (ITGA2B+ITGB3)); (iii) cadherins (Classical (e.g., Cadherin-1 (CDH1), CDH2, CDH3; Desmosomal (e.g., desmoglein (DSG1, DSG2, DSG3, DSG4), desmocollin (DSC1, DSC2, DSC3)); Protocadherin (e.g., PCDH1, PCDH15, PCDH19); Unconventional/ungrouped (e.g., T-cadherin, CDH4, CDH5, CDH6, CDH8, CDH11, CDH12, CDH15, CDH16, CDH17, CDH9, CDH10); (iv) selectins (e.g., E-selectin, L-selectin, P-selectin); and (v) other (e.g., Lymphocyte homing receptor: CD44, L-selectin, integrin (VLA-4, LFA-1), carcinoembryonic antigen (CEA), CD22, CD24, CD44, CD146, CD164).

As used herein, the term “ligand,” refers to any molecule or atom that irreversibly binds to a receiving protein molecule, otherwise known as a receptor. When a ligand binds to its respective receptor, the shape and/or activity of the ligand is altered to initiate several different types of cellular responses. In some aspects, the ligand is a costimulatory ligand. In some aspects, the costimulatory ligand binds to the tumor necrosis factor receptor (TNFR), including OX40 (CD134), 4-1BB (CD137), CD27, glucocorticoid-induced TNFR (GITR; CD357), or CD27. In some aspects, the costimulatory ligand is OX40L, CD137L, CD70, or any combination thereof. In some aspects, the ligand is IL-3 zetakine.

As used herein, the term “enzyme,” refers to the protein that act as biological catalysts by accelerating chemical reactions. The molecules upon which enzymes can act are called substrates, and the enzyme converts the substrates into different molecules known as products. Non-limiting examples of enzymes that can be used in the present methods comprise a prodrug converting enzyme, an IgG-degrading enzyme of S. pyogenes (IdeS), a consensus variant 1 (CV1) protein, matrix metalloproteinase (MMP), heparinase, or any combination thereof.

As used herein, the term “checkpoint inhibitor,” refers to the inhibitor that blocks signaling through the particular immune checkpoint pathway. Non-limiting examples of immune checkpoint inhibitors that can be used in the present methods comprise a CTLA-4 antagonist (e.g., anti-CTLA-4 antibody or antigen binding fragment thereof), PD-1 antagonist (e.g., anti-PD-1 antibody or antigen binding fragment thereof, anti-PD-L1 antibody or antigen binding fragment thereof), TIM-3 antagonist (e.g., anti-TIM-3 antibody or antigen binding fragment thereof), or combinations thereof. In some aspects, the checkpoint inhibitor is a PD-1 antagonist. In some aspects, the checkpoint inhibitor is an anti-PD-1 antibody. In some aspects, the checkpoint inhibitor is an anti-PD-L1 antibody or antigen binding fragment thereof. In some aspects, the checkpoint inhibitor is a TIM-3 antagonist. In some aspects, the checkpoint inhibitor is an anti-TIM-3 antibody or antigen binding fragment thereof.

As used herein, the term “differentiating agent,” refers to an agent that induces differentiation of the mononuclear cells in a cell culture into Committed CIK Precursor cells. Non-limiting examples of differentiating agents that can be used in the present methods comprise IFN-γ, IL-4, IL-5, IL-7, IFN-α, IL-10, IL-12, IL-13, IL-6, IL-15, IL-17, IL-18, IL-21, IL-22, IL-23, IL-27, IL-1β, TGF-β, GM-CSF, CCL3, CCL4, CCL5, CCL17, CCL21, or any combination thereof. In some aspects, the differentiating agent is IFN-γ.

As used herein, the term “stimulating agent,” refers to an agent that supports the stimulation of T cells (e.g., CD3+ cells), before, during or after the transfer of nucleic acids. Non-limiting examples of stimulating agents that can be used in the present methods comprise an anti-CD3 antibody, an anti-TCR antibody, an anti-CD28 antibody, an anti-CD137 antibody, an anti-CD134 antibody, an anti-CD27 antibody, an anti-ICAM-1 antibody, an anti-CD3/CD28-coated beads, a superantigen, phytohaemaglutinin (PHA), phorbol 12-myristate 13-acetate (PMA), ionomycin, or any combination thereof. In some aspects, the stimulating agent is an anti-CD3 antibody. In some aspects, the anti-CD3 antibody is an OKT3 antibody.

As used herein, the term “expanding agent,” refers to an agent that expands cells (e.g., mononuclear cells, such as for example human peripheral blood mononuclear cells (PBMCs)) to generate cells and/or cell populations comprising, for example, the genetically modified CIK cells that express one or more T cell receptors (CIK-TCR), chimeric antigen receptors (CAR-CIK), a genetically modified cell adhesion molecule, a genetically modified ligand, a genetically modified cytokine receptor, a genetically modified chemokine receptor, a genetically modified cytokine, a genetically modified chemokine, an enzyme, or a checkpoint inhibitor. Non-limiting examples of expanding agents that can be used in the present methods IL-2, IL-4, IL-7, IL-9, IL-15, IL-18, IL-21, or any combination thereof. In some aspects, the expanding agent is IL-2.

As used herein, the terms “mononuclear cell,” “mononuclear cells,” and “MNCs” refer to a mixture of different types of cells and contain most of the different stem cells within this component of the marrow, but principally contain a number of immature and mature cell types of different myeloid, lymphoid, and erythroid lineages. Non-limiting examples of mononuclear cells that can be used in the present methods comprise umbilical cord blood derived mononuclear cells, peripheral blood mononuclear cells (PBMCs), bone marrow derived mononuclear cells, lymphocytes, monocytes, dendritic cells, macrophages, T cells, naive T cells, memory T cells, natural killer cells, hematopoietic stem cells, pluripotent embryonic stem cells, induced pluripotent stem cells, or any combination thereof. In some aspects, the mononuclear cells are umbilical cord blood derived mononuclear cells. In some aspects, the mononuclear cells are PBMCs. In some aspects, the PBMCs were previously cryopreserved and thawed before culturing in step (a).

In some aspects, the mononuclear cells are from a human leukocyte antigen (HLA) matched donor.

The term “antigen presenting cells (APCs)” refers to a class of cells capable of presenting one or more antigens in the form of peptide-MHC complex recognizable by specific effector cells of the immune system, and thereby inducing an effective cellular immune response against the antigen or antigens being presented. APCs can be intact whole cells such as macrophages, B-cells, endothelial cells, activated T-cells, and dendritic cells; or other molecules, naturally occurring or synthetic, such as purified MHC Class I molecules complexed to B2-microglobulin. While many types of cells may be capable of presenting antigens on their cell surface for T-cell recognition, only dendritic cells have the capacity to present antigens in an efficient amount to activate naive T-cells for cytotoxic T-lymphocyte (CTL) responses.

The term “immune effector cells” refers to cells capable of binding an antigen and which mediate an immune response. These cells include, but are not limited to, T cells, B cells, monocytes, macrophages, NK cells and cytotoxic T lymphocytes (CTLs), for example CTL lines, CTL clones, and CTLs from tumor, inflammatory, or other infiltrates.

A “naïve” immune effector cell is an immune effector cell that has never been exposed to an antigen capable of activating that cell. Activation of naive immune effector cells requires both recognition of the peptide: HLA complex and the simultaneous delivery of a costimulatory signal by a professional APC in order to proliferate and differentiate into antigen-specific armed effector T cells.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a T_{reg} cell, or activation or inhibition of any other cell of the immune system, e.g., NK cell.

As used herein, the term “educated, antigen-specific immune effector cell,” is an immune effector cell as defined above, which has previously encountered an antigen. In contrast to its naive counterpart, activation of an educated, antigen specific immune effector cell does not require a costimulatory signal. Recognition of the peptide: HLA complex is sufficient.

“Activated,” when used in reference to a T cell, implies that the cell is no longer in Go phase, and begins to produce one or more of cytotoxins, cytokines and other related membrane-associated proteins characteristic of the cell type (e.g., CD8⁺ or CD4⁺), and is capable of recognizing and binding any target cell that displays the particular peptide/HLA complex on its surface, and releasing its effector molecules.

“Immunotherapy” refers to the treatment of a subject afflicted with, or at risk of contracting or suffering a recurrence of, a disease by a method comprising inducing, enhancing, suppressing or otherwise modifying the immune system or an immune response.

“Immuno stimulating therapy” or “immuno stimulatory therapy” refers to a therapy that results in increasing (inducing or enhancing) an immune response in a subject for, e.g., treating cancer.

An increased ability to stimulate an immune response or the immune system, can result from an enhanced agonist activity of T cell co-stimulatory receptors and/or an enhanced antagonist activity of inhibitory receptors. An increased ability to stimulate an immune response or the immune system can be reflected by a fold increase of the EC50 or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity can be enhanced by at least 10%, 30%, 50%, 75%, 2 fold, 3 fold, 5 fold or more.

As used herein, the term “T cell-mediated response” refers to a response mediated by T cells, including effector T cells (e.g., CD8+ cells) and helper T cells (e.g., CD4+ cells). T cell mediated responses include, for example, T cell cytotoxicity and proliferation.

As used herein, the term “cytotoxic T lymphocyte (CTL) response” refers to an immune response induced by cytotoxic T cells. CTL responses are mediated primarily by CD8+ T cells.

The term “autologous” refers to any material derived from the same individual to which it is later to be re-introduced. For example, the tumor antigen that is autologous to the patient comprises administering to a subject the tumor antigen that was isolated from the same subject.

A “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. An example of a cancer that can be treated by the methods of the present disclosure includes, but is not limited to, renal cell cancer. In some aspects, the methods of the present disclosure can be used to reduce the tumor size of a tumor derived from, for example, renal cancer, breast cancer, pancreatic cancer, brain cancer (e.g., astrocytoma, glioblastoma multiforme), bone cancer, prostate cancer, colon cancer, lung cancer, cutaneous or intraocular malignant melanoma, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Waldenström macroglobulinaemia, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia (AML), chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, pituitary adenoma, Kaposi's sarcoma, epidermoid cancer, squamous cell cancer, T-cell lymphoma, environmentally induced cancers including those induced by asbestos, other B cell malignancies, and combinations of said cancers.

The term “tumor” as used herein refers to any mass of tissue that results from excessive cell growth or proliferation, either benign (non-cancerous) or malignant (cancerous), including pre-cancerous lesions.

The term “inflammatory disease” or “inflammatory disorder” refers to a medical condition at least partially characterised by inappropriate secretion of inflammatory mediators (e.g. highly toxic reactive oxygen intermediates (ROIs) or granule enzymes or cytokines) from granulocytes into an affected tissue. Examples of such conditions include, but are not limited to, rheumatoid arthritis, Behcet's disease, Anti-Neutrophil Cytoplasmic Antibody (ANCA)-associated vasculitis, systemic vasculitis, cystic fibrosis, asthma, Crohn's Disease, multiple sclerosis, autoimmune tyroiditis, diabetes mellitus (Juvenile onset diabetes), autoimmune uveoretinitis, myasthenia gravis, systemic lupus erythematosus (SLE), Sjögren's syndrome, celiac disease, alopecia, irritable bowel syndrome (IBS), psoriasis, and any combination thereof.

The term “autoimmune disease” or “autoimmune disorder” refers to diseases and disorders that occur as a result of the immune system attacking the body's own organs, tissues, and cells. Autoimmune diseases in mammals can generally be classified in one of two different categories: cell-mediated disease (i.e., T-cell) or antibody-mediated disorders. Non-limiting examples of cell-mediated autoimmune diseases include multiple sclerosis, rheumatoid arthritis, autoimmune tyroiditis, diabetes mellitus (Juvenile onset diabetes), and autoimmune uveoretinitis. Antibody-mediated autoimmune disorders include myasthenia gravis and systemic lupus erythematosus (or SLE).

The term “viral infection” refers to an infection from e.g., Classes I through V viruses. As used herein, the term “Class I-V viruses” refers to the different classes of virus identified by genome composition and strategy for mRNA synthesis, as described in Lodish, H. et al., Molecular Cell Biology, Fourth Edition, W. H. Freeman and Company (2000). Class I-V viruses are identified as follows:
Class I viruses contain a single molecule of double-stranded DNA;
Class II viruses contain a single molecule of single-stranded DNA;
Class III viruses contain double-stranded genomic RNA;
Class IV viruses contain a single strand of viral mRNA (also known as a positive/plus strand of genomic RNA), wherein the viral mRNA encodes proteins and is infectious by itself; and
Class V viruses contain a single strand of an RNA sequence that is complimentary to the genomic viral mRNA (also known as a negative/minus strand of genomic RNA), wherein the genomic RNA acts as a template for synthesis of mRNA but does not itself encode proteins.

Non-limiting examples of viruses that can treated by the present invention include arboviruses (including but not limited to dengue virus, yellow fever, etc.); adenoviruses (acute respiratory disease, pneumonia, conjunctivitis, gastroenteritis, pharynx) Inflammation, acute hemorrhagic cystitis, African swine fever, swine circovirus, swine adenovirus type A, type B, and type C); herpes virus [herpes simplex virus, varicella-zoster virus (Including, but not limited to, chicken pox and shingles), Epstein-Barr virus; human papillomavirus (including but not limited to HPV types 1-65); parvovirus (parvovirus B19, canine parvovirus) Including but not limited to: reovirus (orbivirus, rotavirus) Including, but not limited to, aquareovirus, cortivirus; picornavirus (including but not limited to enterovirus, rhinovirus, hepatovirus); coronavirus (including but not limited to coronavirus and torovirus) Flaviviruses (including but not limited to pestiviruses, hepatitis C-like viruses); togaviruses (including but not limited to alphaviruses and rubyviruses), orthomyxoviruses (influenzas) Including, but not limited to, A, B, and C viruses, avian influenza viruses, and togoviruses; Bunyaviruses (including but not limited to hantaviruses, nairoviruses, and levoviruses); Viruses (including but not limited to, rabies virus, ephemerovirus, becyclovirus); and paramyxoviruses (including but not limited to measles virus and mumps virus).

As used herein, the term a “composition” refers to a combination of active agent and another compound or composition, inert (for example, a detectable agent or label) or active, such as an adjuvant.

As used herein, the term a “pharmaceutical composition” as described herein refers to the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin REMINGTON'S PHARM. SCI., 18th Ed. (Mack Publ. Co., Easton (1990)).

As used herein, the term “formulating” refers to formulating transfected cells comprising the Committed CIK Precursor cells, as described herein, that are recovered post electroporation. In some aspects, transfected cells comprising the Committed CIK Precursor cells are for administration to the subject. In some aspects, transfected cells comprising the Committed CIK Precursor cells are formulated in cryopreservation media.

As used herein, “administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion.

The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation.

In some aspects, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject,” “patient,” “individual,” and “host” are used interchangeably herein. As used herein, the phrase “subject in need thereof” includes subjects, such as mammalian subjects, that would benefit, e.g., from administration of formulated transfected cells comprising the Committed CIK Precursor cells cultured using the methods provided herein, as described herein.

The term “therapeutically effective amount” or “therapeutically effective dosage” refers to an amount of an agent (e.g., formulated transfected cells comprising the Committed CIK Precursor cells cultured as described herein) that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. For example, in reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations.

The effective amount of the composition (e.g., formulated transfected cells comprising the Committed CIK Precursor cells cultured as described herein) can, for example, (i) reduce the number of cancer cells; (ii) reduce tumor size; (iii) inhibit, delay, slow to some extent and can stop cancer cell infiltration into peripheral organs; (iv) inhibit (i.e., slow to some extent and can stop tumor metastasis); (v) inhibit tumor growth; (vi) prevent or delay occurrence and/or recurrence of tumor; and/or (vii) relieve to some extent one or more of the symptoms associated with the cancer.

In some aspects, a “therapeutically effective amount” is the amount of a composition disclosed herein (e.g., formulated transfected cells comprising the Committed CIK Precursor cells cultured as described herein), which is clinically proven to effect a significant decrease in cancer or slowing of progression (regression) of cancer, such as an advanced solid tumor. The ability of a therapeutic agent of the present disclosure (e.g., formulated transfected cells comprising the Committed CIK Precursor cells cultured as described herein) to promote disease regression can be evaluated using a variety of methods known to the skilled practitioner, such as in human subjects during clinical trials, in animal model systems predictive of efficacy in humans, or by assaying the activity of the agent in in vitro assays.

The terms “effective” and “effectiveness” with regard to a treatment include both pharmacological effectiveness and physiological safety. Pharmacological effectiveness refers to the ability of a composition disclosed herein (e.g., immune cells modified and cultured as described herein) to promote cancer regression in the patient. Physiological safety refers to the level of toxicity, or other adverse physiological effects at the cellular, organ, and/or organism level (adverse effects) resulting from administration of a composition disclosed herein (e.g., immune cells modified and cultured as described herein).

The terms “cytokine-induced killer cell,” “cytokine-induced killer cells,” “CIK cell” and “CIK cells” as used herein, refer to multi functional cells that can express both T cell and natural killer T (NKT) cells markers (e.g., CD3+CD56+). Upon administration of the CIK cells into the patient, the terminally differentiated CD3− and CD56−positive subset of the CIK cells primarily exert the direct HLA-unrestricted tumor killing activity.

As used herein, the terms “committed cytokine-induced killer precursor cells,” “committed cytokine induced killer precursor cells,” and “committed CIK precursor cells” refer to activated day 2 cells derived from peripheral blood mononuclear cells (PBMCs) by the sequential addition of one or more cytokines, such as IFN-γ on day 0, and then stimulation through the CD3 receptor with the addition of IL-2 on day 1. Based on the fact that activated day 2 cells still have high CD28 and CCR7 together with the fact that the cells did not yet upregulate CD56 and NKG2D markers, they cannot be called cytokine induced killer (CIK) cells yet. However, these activated day 2 cells no longer require any modification with additional stimuli and are committed to CIK phenotype during their proliferation.

The terms “chimeric antigen receptor” and “CAR,” as used herein, refer to a set of polypeptides, typically two in the simplest form, which when in an immune effector cell, provides the cell with specificity for a target cell, typically a cancer cell, and with intracellular signal generation. In some aspects, a CAR comprises at least an extracellular antigen-binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspects, the set of polypeptides are in the same polypeptide chain, e.g., comprise a chimeric fusion protein. In some aspects, the set of polypeptides are not contiguous with each other, e.g., are in different polypeptide chains. In some aspects, the set of polypeptides include a dimerization switch that, upon the presence of a dimerization molecule, can couple the polypeptides to one another, e.g., can couple an antigen-binding domain to an intracellular signaling domain. In some aspects, the stimulatory molecule of the CAR is the zeta chain associated with the T cell receptor complex (e.g., CD3 zeta). In some aspects, the cytoplasmic signaling domain comprises a primary signaling domain (e.g., a primary signaling domain of CD3-zeta). In some aspects, the cytoplasmic signaling domain further comprises one or more functional signaling domains derived from at least one costimulatory molecule as defined below. In some aspects, the costimulatory molecule is chosen from the costimulatory molecules described herein, e.g., 4-1BB (i.e., CD137), CD27, and/or CD28.

In some aspects, the CAR comprises a chimeric fusion protein comprising an antigen-binding domain, a transmembrane domain, and an intracellular signaling domain comprising a functional signaling domain derived from a stimulatory molecule, wherein the antigen-binding domain and the transmembrane domain are linked by a CAR spacer. In some aspects, the CAR comprises a chimeric fusion protein comprising an antigen-binding domain linked to a transmembrane domain via a CAR spacer and an intracellular signaling domain comprising a functional signaling domain derived from a costimulatory molecule and a functional signaling domain derived from a stimulatory molecule. In some aspects, the CAR comprises a chimeric fusion protein comprising an antigen-binding domain linked to a transmembrane domain via a CAR spacer and an intracellular signaling domain comprising two functional signaling domains derived from one or more costimulatory molecule(s) and a functional signaling domain derived from a stimulatory molecule. In some aspects, the CAR comprises a chimeric fusion protein comprising an antigen-binding domain linked to a transmembrane domain via a CAR spacer and an intracellular signaling domain comprising at least two functional signaling domains derived from one or more costimulatory molecule(s) and a functional signaling domain derived from a stimulatory molecule. In some aspects, the CAR comprises an optional leader sequence at the amino-terminus (N-terminus) of the CAR. In some aspects, the CAR further comprises a leader sequence at the N-terminus of the antigen-binding domain, wherein the leader sequence is optionally cleaved from the antigen-binding domain (e.g., a scFv) during cellular processing and localization of the CAR to the cellular membrane.

The antigen-specific extracellular domain of a chimeric antigen receptor recognizes and specifically binds an antigen, typically a surface-expressed antigen of a malignancy. An antigen-specific extracellular domain specifically binds an antigen when, for example, it binds the antigen with an affinity constant or affinity of interaction (K_{D}) between about 0.1 pM to about 10 μM, for example, about 0.1 pM to about 1 μM or about 0.1 pM to about 100 nM. Methods for determining the affinity of interaction are known in the art. An antigen-specific extracellular domain suitable for use in a CAR of the present disclosure can be any antigen-binding polypeptide, a wide variety of which are known in the art. In some aspects, the antigen-binding domain is a single chain Fv (scFv). Other antibody-based recognition domains such as cAb VHH (camelid antibody variable domains) and humanized versions thereof, 1gNAR VH (shark antibody variable domains) and humanized versions thereof, sdAb VH (single domain antibody variable domains), and “camelized” antibody variable domains are also suitable for use in a CAR of the present disclosure. In some aspects, T cell receptor (TCR) based recognition domains, such as single chain TCR (scTv, i.e., single chain two-domain TCR containing VaVB) are also suitable for use in the chimeric binding proteins of the present disclosure.

As used herein, the term “T cell receptor” or “TCR” refers to a heterodimer composed of 2 different transmembrane polypeptide chains: an α chain and a β chain, each consisting of a constant region, which anchors the chain inside the T-cell surface membrane, and a variable region, which recognizes and binds to the antigen presented by HLAs. The TCR complex is associated with 6 polypeptides forming 2 heterodimers, CD3γε and CD3δε, and 1 homodimer CD3 ζ, which together forms the CD3 complex. T-cell receptor-engineered T-cell therapy utilizes the modification of T cells that retain these complexes to specifically target the antigens expressed by particular tumor cells. As used herein, the term “TCR” includes naturally occurring TCRs and engineered TCRs.

A “TCR mimic” or a “TCRm” refers to a type of antibody that recognize epitopes comprising both the peptide and the MHC-I molecule, similar to the recognition of such complexes by the TCR on T cells.

The term “genetically modified” refers to containing and/or expressing a foreign gene or nucleic acid sequence which in turn, modifies the genotype or phenotype of the cell or its progeny. In other words, it refers to any addition, deletion or disruption to a cell's endogenous nucleotides.

The terms “nucleic acids,” “nucleic acid molecules, “nucleotides,” “nucleotide(s) sequence,” and “polynucleotide” can be used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein. As described herein, in some aspects, a polynucleotide of the present disclosure can comprise a single nucleotide sequence encoding a single protein. In some aspects, a polynucleotide of the present disclosure is polycistronic (i.e., comprises two or more cistrons). In some aspects, each of the cistrons of a polycistronic polynucleotide can encode for a protein disclosed herein. In some aspects, each of the cistrons can be translated independently of one another.

As used herein, the term “polypeptide” encompasses both peptides and proteins, unless indicated otherwise. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

As used herein, the term “fragment” of a polypeptide refers to an amino acid sequence of a polypeptide that is shorter than the naturally-occurring sequence, N- and/or C-terminally deleted or any part of the polypeptide deleted in comparison to the naturally occurring polypeptide. Thus, a fragment does not necessary need to have only N- and/or C-terminal amino acids deleted. A polypeptide in which internal amino acids have been deleted with respect to the naturally occurring sequence is also considered a fragment.

As used herein, the term “functional fragment” or “functional portion” refers to a polypeptide fragment that retains polypeptide function. Accordingly, in some aspects, a functional fragment of an Ig hinge, retains the ability to position an antigen-binding domain (e.g., an scFv) in a chimeric binding protein at a distance from a target epitope (e.g., a tumor antigen) such that the antigen-binding domain (e.g., an scFv) can effectively interact with the target epitope (e.g., a tumor antigen).

A “recombinant” polypeptide or protein refers to a polypeptide or protein produced via recombinant DNA technology. Recombinantly produced polypeptides and proteins expressed in engineered host cells are considered isolated for the purpose of the disclosure, as are native or recombinant polypeptides which have been separated, fractionated, or partially or substantially purified by any suitable technique. The polypeptides encoded by the polynucleotides disclosed herein (e.g., chimeric binding protein) can be recombinantly produced using methods known in the art. In some aspects, the polypeptides encoded by the polynucleotides of the present disclosure (e.g., chimeric binding protein) are produced by cells, e.g., T cells, following transfection or modification with at least one polynucleotide or vector encoding the polypeptides described here.

As used herein, a “coding region,” “coding sequence,” or “translatable sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide.

The terms “complementary” and “complementarity” refer to two or more oligomers (i.e., each comprising a nucleobase sequence), or between an oligomer and a target gene, that are related with one another by Watson-Crick base-pairing rules. For example, the nucleobase sequence “T-G-A (5′ to 3′),” is complementary to the nucleobase sequence “A-C-T (3′ to 5′).” Complementarity can be “partial,” in which less than all of the nucleobases of a given nucleobase sequence are matched to the other nucleobase sequence according to base pairing rules. For example, in some aspects, complementarity between a given nucleobase sequence and the other nucleobase sequence can be about 70%, about 75%, about 80%, about 85%, about 90%, or about 95%. Accordingly, in some aspects, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a target nucleic acid sequence (e.g., miR-485 nucleic acid sequence). Or, there can be “complete” or “perfect” (100%) complementarity between a given nucleobase sequence and the other nucleobase sequence to continue the example. In some aspects, the degree of complementarity between nucleobase sequences has significant effects on the efficiency and strength of hybridization between the sequences.

The term “expression” as used herein refers to a process by which a polynucleotide produces a gene product, for example, a polypeptide. It includes, without limitation, transcription of the polynucleotide into messenger RNA (mRNA) and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

“Gene delivery,” “gene transfer,” “transfection” and the like, as used herein, are terms referring to the introduction of an exogenous polynucleotide into a host cell, irrespective of the method used for the introduction. Transfection refers to delivery of any nucleic acid to the interior of a cell. Gene delivery refers to the delivery of a nucleic acid that can be integrated into the host cell's genome, or that can replicate independently of the host cell genome. Transfection methods include a variety of techniques such as electroporation, protein-based, lipid-based and cationic ion based nucleic acid delivery complexes, viral vectors, “gene gun” delivery and various other techniques known to those of skill in the art. The introduced polynucleotide can be stably maintained in the host cell or can be transiently expressed. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are capable of mediating transfer of genes to mammalian cells, as is known in the art.

As used herein, the term “electroporation” refers to a physical transfection method that permeabilizes the cell membrane by applying an electrical pulse and moves molecules via the electrical field into the cell.

As used herein, the term “identity” refers to the overall monomer conservation between polymeric molecules, e.g., between polynucleotide molecules. The term “identical” without any additional qualifiers, e.g., polynucleotide A is identical to polynucleotide B, implies the polynucleotide sequences are 100% identical (100% sequence identity). Describing two sequences as, e.g., “70% identical,” is equivalent to describing them as having, e.g., “70% sequence identity.” A “reference nucleotide sequence,” when used herein as a comparison to a nucleotide sequence of the disclosure, refers to a polynucleotide sequence essentially identical to the nucleotide sequence of the disclosure except that sequence is not optimized.

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In some aspects, the length of a sequence aligned for comparison purposes is at least about 30%, at least about 40%, at least about 50%, at least about 60%, at least about 70%, at least about 80%, at least about 90%, at least about 95%, or about 100% of the length of the reference sequence. The amino acids at corresponding amino acid positions, or bases in the case of polynucleotides, are then compared.

When a position in the first sequence is occupied by the same amino acid or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs that can be used to align different sequences (e.g., polynucleotide sequences) are available from various sources. One suitable program to determine percent sequence identity is b12seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In some aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at tcoffee.org, and alternatively available, e.g., from the EBI. It will also be appreciated that the final alignment used to calculate percent sequence identity can be curated either automatically or manually.

As used herein, the terms “isolated,” “purified,” “extracted,” and grammatical variants thereof are used interchangeably and refer to the state of a preparation of desired composition of the present disclosure that has undergone one or more processes of purification. In some aspects, isolating or purifying as used herein is the process of removing, partially removing (e.g., a fraction) of a composition of the present disclosure.

In some aspects, an isolated composition has no detectable undesired activity or, alternatively, the level or amount of the undesired activity is at or below an acceptable level or amount. In some aspects, an isolated composition has an amount and/or concentration of desired composition of the present disclosure, at or above an acceptable amount and/or concentration and/or activity. In some aspects, the isolated composition is enriched as compared to the starting material from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material.

In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first polynucleotide sequence to a second polynucleotide sequence at the 5′-end or the 3′-end, but also includes insertion of the whole first polynucleotide sequence (or the second polynucleotide sequence) into any two nucleotides in the second polynucleotide sequence (or the first polynucleotide sequence, respectively). The first polynucleotide sequence can be linked to a second polynucleotide sequence by a phosphodiester bond or a linker. The linker can be, e.g., a polynucleotide.

“Administering” (and grammatical variants thereof) refers to the physical introduction of a therapeutic agent (e.g., an engineered cell described herein) to a subject, using any of the various methods and delivery systems known to those skilled in the art. Exemplary routes of administration include intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural, intrasterna, oral, rectal, topical, epidermal, mucosal, intranasal, vaginal, rectal, sublingual administration, and combinations thereof. Administering can also be performed, for example, once, a plurality of times, and/or over one or more extended periods.

“Treatment” or “therapy” (including any grammatical derivatives thereof) of a subject refers to any type of intervention or process performed on, or the administration of an active agent to, a subject with the objective of reversing, alleviating, ameliorating, inhibiting, slowing down, or preventing the onset, progression, development, severity, or recurrence of a symptom, complication, condition, or biochemical indicia associated with a disease. In some aspects, the term refers to inducing an immune response in a subject against an antigen.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

As used herein the term “therapeutically effective amount” is the amount of reagent or pharmaceutical compound comprising a composition disclosed herein (e.g., modified immune cell described herein) that is sufficient to a produce a desired therapeutic effect, pharmacologic and/or physiologic effect on a subject in need thereof.

A therapeutically effective amount can be a “prophylactically effective amount” as prophylaxis can be considered therapy. As used herein, “prophylactic” refers to a therapeutic or course of action used to prevent the onset of a disease or condition, or to prevent or delay a symptom associated with a disease or condition. As used herein, a “prophylaxis” refers to a measure taken to maintain health and prevent the onset of a disease or condition, or to prevent or delay a symptom associated with a disease or condition.

As used herein, the term “promoter” refers to DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

As used herein, a solution, e.g., buffer, is considered “isotonic” when the concentration of solutes in the buffer is equivalent to the concentration of solutes inside the cell.

As used herein, the terms “ug” and “uM” are used interchangeably with “μg” and “μM,” respectively.

Various aspects described herein are described in further detail in the following subsections.

### II. Methods of the Disclosure

The present disclosure is directed to methods of generating genetically modified cytokine induced killer (CIK) cells, comprising the sequential steps of:
(a) culturing a population of mononuclear cells in culture medium comprising at least one differentiating agent to induce differentiation of the mononuclear cells in a cell culture into Committed CIK Precursor cells;
(b) adding at least one stimulating agent and at least one expanding agent to the cell culture; and
(c) expanding the cells from the cell culture to obtain a cell population comprising Committed CIK Precursor cells, transfecting the cell population comprising the Committed CIK Precursor cells with one or more nucleic acids to produce genetically modified Committed CIK Precursor cells, and expanding the genetically modified Committed CIK Precursor cells in culture medium to produce the genetically modified CIK cells;
wherein steps (a), (b) and (c) are performed in the absence of non-irradiated or irradiated feeder cells.

The present disclosure is also directed methods of of generating genetically modified cytokine induced killer (CIK) cells, comprising the sequential steps of:
(a) culturing a population of mononuclear cells in culture medium comprising at least one differentiating agent to induce differentiation of the mononuclear cells in a cell culture into Committed CIK Precursor cells;
(b) adding at least one stimulating agent and at least one expanding agent to the cell culture;
(c) expanding the cells from the cell culture to obtain a cell population comprising Committed CIK Precursor cells and transfecting the cell population comprising the Committed CIK Precursor cells with one or more nucleic acids;
(d) formulating transfected cells comprising the Committed CIK Precursor cells for administration to a subject in need thereof; and
(e) administering the transfected cells to the subject, wherein transfected Committed CIK Precursor cells are expanded to produce the genetically modified CIK cells in the subject;
wherein steps (a), (b) and (c) are performed in the absence of non-irradiated or irradiated feeder cells.

The present disclosure is directed to methods of generating genetically modified cytokine induced killer (CIK) cells, comprising the sequential steps of:
(a) culturing peripheral blood mononuclear cells (PBMCs) in culture medium comprising at least one differentiating agent to induce differentiation of the PBMCs in a cell culture into Committed CIK Precursor cells;
(b) adding at least one stimulating agent and at least one expanding agent to the cell culture; and
(c) expanding the cells from the cell culture to obtain a cell population comprising Committed CIK Precursor cells, transfecting the cell population comprising the Committed CIK Precursor cells with one or more nucleic acids to produce genetically modified Committed CIK Precursor cells, and expanding the genetically modified Committed CIK Precursor cells in culture medium to produce the genetically modified CIK cells;
wherein steps (a), (b) and (c) are performed in the absence of non-irradiated or irradiated feeder cells; and
wherein the PBMCs were previously cryopreserved and thawed before culturing in step (a).

The present disclosure is also directed methods of of generating genetically modified cytokine induced killer (CIK) cells, comprising the sequential steps of:
(a) culturing peripheral blood mononuclear cells (PBMCs) in culture medium comprising at least one differentiating agent to induce differentiation of the PBMCs in a cell culture into Committed CIK Precursor cells;
(b) adding at least one stimulating agent and at least one expanding agent to the cell culture;
(c) expanding the cells from the cell culture to obtain a cell population comprising Committed CIK Precursor cells and transfecting the cell population comprising the Committed CIK Precursor cells with one or more nucleic acids;
(d) formulating transfected cells comprising the Committed CIK Precursor cells for administration to a subject in need thereof; and
(e) administering the transfected cells to the subject, wherein transfected Committed CIK Precursor cells are expanded to produce the genetically modified CIK cells in the subject;
wherein steps (a), (b) and (c) are performed in the absence of non-irradiated or irradiated feeder cells; and
wherein the PBMCs were previously cryopreserved and thawed before culturing in step (a).

### II.A Differentiating Agents

In some aspects, the differentiating agent is selected from the group consisting of: IFN-γ, IL-4, IL-5, IL-7, IFN-α, IL-10, IL-12, IL-13, IL-6, IL-15, IL-17, IL-18, IL-21, IL-22, IL-23, IL-27, IL-1β, TGF-β, GM-CSF, CCL3, CCL4, CCL5, CCL17, CCL21, and any combination thereof.

In some aspects, the differentiating agent is IFN-γ.

In some aspects, the differentiating agent is added in an amount of about 10 U/ml to about 10000 U/ml.

In some aspects, the differentiating agent is added in an amount of about 10 U/ml, about 20 U/ml, about 30 U/ml, about 40 U/ml, about 50 U/ml, about 60 U/ml, about 70 U/ml, about 80 U/ml, about 90 U/ml, about 100 U/ml, about 200 U/ml, about 300 U/ml, about 400 U/ml, about 500 U/ml, about 600 U/ml, about 700 U/ml, about 800 U/ml, about 900 U/ml, about 1000 U/ml, about 2000 U/ml, about 3000 U/ml, about 4000 U/ml, about 5000 U/ml, about 6000 U/ml, about 7000 U/ml, about 8000 U/ml, about 9000 U/ml, or about 10000 U/ml.

In some aspects, the differentiating agent is added in an amount of about 1000 U/ml.

### II.B Expanding and Stimulating Agents

In some aspects, the stimulating agent is selected from the group consisting of: an anti-CD3 antibody, an anti-TCR antibody, an anti-CD28 antibody, an anti-CD137 antibody, an anti-CD134 antibody, an anti-CD27 antibody, an anti-ICAM-1 antibody, an anti-CD3/CD28-coated beads, a superantigen, phytohaemaglutinin (PHA), phorbol 12-myristate 13-acetate (PMA), ionomycin, and any combination thereof.

In some aspects, the stimulating agent is an anti-CD3 antibody. In some aspects, the anti-CD3 antibody is an OKT3 antibody.

In some aspects, the stimulating agent is added in an amount of about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 31 ng/ml, about 32 ng/ml, about 33 ng/ml, about 34 ng/ml, about 35 ng/ml, about 36 ng/ml, about 37 ng/ml, about 38 ng/ml, about 39 ng/ml, about 40 ng/ml, about 41 ng/ml, about 42 ng/ml, about 43 ng/ml, about 44 ng/ml, about 45 ng/ml, about 46 ng/ml, about 47 ng/ml, about 48 ng/ml, about 49 ng/ml, about 50 ng/ml, about 51 ng/ml, about 52 ng/ml, about 53 ng/ml, about 54 ng/ml, about 55 ng/ml, about 56 ng/ml, about 57 ng/ml, about 58 ng/ml, about 59 ng/ml, about 60 ng/ml, about 61 ng/ml, about 62 ng/ml, about 63 ng/ml, about 64 ng/ml, about 65 ng/ml, about 66 ng/ml, about 67 ng/ml, about 68 ng/ml, about 69 ng/ml, about 70 ng/ml, about 71 ng/ml, about 72 ng/ml, about 73 ng/ml, about 74 ng/ml, about 75 ng/ml, about 76 ng/ml, about 77 ng/ml, about 78 ng/ml, about 79 ng/ml, about 80 ng/ml, about 81 ng/ml, about 82 ng/ml, about 83 ng/ml, about 84 ng/ml, about 85 ng/ml, about 86 ng/ml, about 87 ng/ml, about 88 ng/ml, about 89 ng/ml, about 90 ng/ml, about 91 ng/ml, about 92 ng/ml, about 93 ng/ml, about 94 ng/ml, about 95 ng/ml, about 96 ng/ml, about 97 ng/ml, about 98 ng/ml, about 99 ng/ml, or about 100 ng/ml.

In some aspects, the stimulating agent is added in an amount of about 50 ng/ml.

In some aspects, the expanding agent is selected from the group consisting of: IL-2, IL-4, IL-7, IL-9, IL-15, IL-18, IL-21, and any combination thereof.

In some aspects, the expanding agent is IL-2.

In some aspects, the expanding agent is added in an amount of about 10 U/ml to about 1000 U/ml.

In some aspects, the expanding agent is added in an amount of about 300 U/ml.

In some aspects, the stimulating agent and the expanding agent are added to the cell culture approximately 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours after initiating step (a).

In some aspects, the stimulating agent and the expanding agent are added to the cell culture approximately 18 hours after initiating step (a).

In some aspects, the stimulating agent and the expanding agent are added to the cell culture 18 hours after initiating step (a).

In some aspects, the stimulating agent and the expanding agent are added to the cell culture approximately 19 hours after initiating step (a).

In some aspects, the stimulating agent and the expanding agent are added to the cell culture 19 hours after initiating step (a).

In some aspects, the stimulating agent and the expanding agent are added to the cell culture approximately 20 hours after initiating step (a).

In some aspects, the stimulating agent and the expanding agent are added to the cell culture 20 hours after initiating step (a).

In some aspects, the stimulating agent and the expanding agent are added to the cell culture approximately 21 hours after initiating step (a).

In some aspects, the stimulating agent and the expanding agent are added to the cell culture 21 hours after initiating step (a).

In some aspects, the stimulating agent and the expanding agent are added to the cell culture approximately 22 hours after initiating step (a).

In some aspects, the stimulating agent and the expanding agent are added to the cell culture 22 hours after initiating step (a).

In some aspects, the stimulating agent and the expanding agent are added to the cell culture approximately 23 hours after initiating step (a).

In some aspects, the stimulating agent and the expanding agent are added to the cell culture 23 hours after initiating step (a).

In some aspects, the stimulating agent and the expanding agent are added to the cell culture approximately 24 hours after initiating step (a).

In some aspects, the stimulating agent and the expanding agent are added to the cell culture 24 hours after initiating step (a).

### II.C Transfection/Cell Culture

In some aspects, the Committed CIK Precursor cells are transfected with one or more nucleic acids approximately 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours after initiating step (b).

In some aspects, the Committed CIK Precursor cells are transfected with one or more nucleic acids approximately 18 hours after initiating step (b).

In some aspects, the Committed CIK Precursor cells are transfected with one or more nucleic acids approximately 19 hours after initiating step (b).

In some aspects, the Committed CIK Precursor cells are transfected with one or more nucleic acids approximately 20 hours after initiating step (b).

In some aspects, the Committed CIK Precursor cells are transfected with one or more nucleic acids approximately 21 hours after initiating step (b).

In some aspects, the Committed CIK Precursor cells are transfected with one or more nucleic acids approximately 22 hours after initiating step (b).

In some aspects, the Committed CIK Precursor cells are transfected with one or more nucleic acids approximately 23 hours after initiating step (b).

In some aspects, the Committed CIK Precursor cells are transfected with one or more nucleic acids approximately 24 hours after initiating step (b).

In some aspects, the cell population comprising the Committed CIK Precursor cells is transfected using electroporation.

In some aspects, the electroporation in performed in an isotonic buffer.

In some aspects, the electroporation in performed in CoStorSol (Preservation Solutions Inc).

In some aspects, the electroporation in performed in Opti-MEM.

In some aspects, the electroporation in performed in Lonza isotonic buffer (cat #PBP3-02250).

In some aspects, the transfection is selected from the group consisting of: a non-viral transfer of one or more nucleic acids encoding an antigen receptor, a chimeric antigen receptor, a T cell receptor, a suicide gene, a gene encoding an inducible caspase 9 system, and any combination thereof into the cell population comprising the Committed CIK Precursor cells in the cell culture.

In some aspects, the non-viral transfer of nucleic acids comprises the use of the group consisting of: a transposon-based integration system, Zn-finger nucleases, integrases, transcription activator-like effectors, clustered regularly interspaced short palindromic repeats (CRISPR), sequence-specific recombinase systems able to integrate nucleic acids by recombination between attachment sites, and any combination thereof.

In some aspects, the transposon-based system is a Sleeping Beauty (SB) transposon-based system.

In some aspects, the SB transposon-based system comprises the use of Sleeping Beauty transposase SB100X (see e.g., Hudecek et al., Crit. Rev. in Biochem and Mol Bio 52(4):355-380 (2017)).

In some aspects, the Committed CIK Precursor cells are transfected with an RNA encoding SB100X transposase and a DNA encoding a Sleeping Beauty compatible chimeric antigen receptor (CAR) transposon.

In some aspects, one or more nucleic acids encode T cell receptors, chimeric antigen receptors, an adhesion molecule, a costimulatory ligand, a cytokine receptor, a chemokine receptor, a cytokine, a chemokine, an enzyme, a secreted drug, a checkpoint inhibitor, or a ligand.

In some aspects, one or more nucleic acids encode a prodrug converting enzyme, an IgG-degrading enzyme of S. pyogenes (IdeS), a consensus variant 1 (CV1) protein, Intercellular Adhesion Molecule 1 (ICAM-1), CD137L, OX40L, CD70, IL-15 receptor, CCR5, CCR4, CD25, CD122, CD132, C-X-C chemokine receptor type 4 C-X-C chemokine receptor type 4 (CXCR4), IL-15, IL-18, IL-21 IL-23 IL-33, IL-1a, IL-1b, matrix metalloproteinase (MMP), heparinase, an anti-PD-1 antibody or antigen binding fragment thereof, an anti-T cell immunoglobulin and mucin-domain containing-3 (TIM-3) antibody or antigen binding fragment thereof, IL-3 zetakine, or any combination thereof.

In some aspects, the prodrug converting enzyme is carboxypeptidase G2 (CPG2) or β-lactamase.

In some aspects, the chimeric antigen receptors are specific for CD19, CD123, TIM-3, C-type lectin-like molecule-1 (CLL-1), CD70, mucin 1 (MUC-1), CD20, CD22, B-cell activating factor receptor (BAFFR), CD23, cytokine receptor like factor 2 (CRLF2), CD79b, CD79d, CD7, CD43, CD5, CD25, Lewis Y (LeY), natural killer group 2 member D (NKG2D), receptor tyrosine kinase like orphan receptor 1 (ROR1), receptor tyrosine kinase like orphan receptor 2 (ROR2), Wilms' tumor 1 (WT1), CD44 variant 6 (CD44v6), CD33, CD3ε, human epidermal growth factor receptor 2 (Her2), epidermal growth factor receptor (EGFR), epidermal growth factor receptor variant III (EGFRvIII), CA125, CD138, prostate-specific membrane antigen (PSMA), B7 homolog 3 protein (B7-H3), CD30, disialoganglioside GD-2, disialoganglioside GD-3, CD171, mesothelin (MSLN), ephrin type-A receptor 2 (EphA2), carcinoembryonic antigen (CEA), vascular endothelial growth factor receptor (VEGFR), IL13Rα2, prostate stem cell antigen (PSCA), epithelial cellular adhesion molecule (EpCAM), chondroitin sulfate proteoglycan 4 (CSPG4), folate receptor alpha (FRα), fibroblast activation protein (FAP), carbonic anhydrase IX (CAIX), B-cell maturation antigen (BCMA), signaling lymphocytic activation molecule family member 7 (SLAM7), CD126, mesenchymal-epithelial transition factor (c-MET), AXL receptor tyrosine kinase (AXL), CD133, tumor endothelial marker 8 (TEM8), tumor-associated calcium signal transducer 2 (TROP2), programmed death-ligand 1 (PD-L1), delta-like ligand 3 (DLL3), melanoma-associated antigen 1 (MAGE-1), protein tyrosine kinase 7 (PTK7), c-type lectin domain containing 14A (CLEC14A), C-X-C chemokine receptor type 4 (CXCR4), New York esophageal squamous cell carcinoma 1 (NY-ESO-1) CD126, tumor-associated glycoprotein 72 (TAG-72), Guanylate cyclase 2C (GUCY2C), Cadherin-6 (CDH6), cadherin 17 (CDH17), claudin18.2 (CLDN18.2), glypican-3 (GPC3), CD147, CD16, glycoprotein 100 (gp100)/human leukocyte antigen-A2 (HLA-A2) complex, MACEP, urokinase-type plasminogen activator receptor (uPAR), alkaline phosphatase, placental-like 2 (ALPPL2), CD47, olfactory receptor 2H1 (OR2H1), matrix metalloproteinase-2 (MMP-2), podoplanin (PDPN), GDNF family receptor alpha-4 (GFRα4), thyrotropin receptor (TSHR), L1 cell adhesion molecule (L1-CAM), or any combination thereof.

In some aspects, the chimeric antigen receptor is specific for CD19.

In some aspects, one or more nucleic acids are DNA and/or RNA.

In some aspects, one or more nucleic acids are RNA.

In some aspects, the methods disclosed herein further comprise (d) replacing a portion of the culture medium with a fresh culture medium comprising at least one expanding agent approximately 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after initiating step (c).

In some aspects, the methods disclosed herein further comprise (d) replacing a portion of the culture medium with a fresh culture medium comprising at least one expanding agent approximately 18 hours after initiating step (c).

In some aspects, the methods disclosed herein further comprise (d) replacing a portion of the culture medium with a fresh culture medium comprising at least one expanding agent approximately 19 hours after initiating step (c).

In some aspects, the methods disclosed herein further comprise (d) replacing a portion of the culture medium with a fresh culture medium comprising at least one expanding agent approximately 20 hours after initiating step (c).

In some aspects, the methods disclosed herein further comprise (d) replacing a portion of the culture medium with a fresh culture medium comprising at least one expanding agent approximately 21 hours after initiating step (c).

In some aspects, the methods disclosed herein further comprise (d) replacing a portion of the culture medium with a fresh culture medium comprising at least one expanding agent approximately 22 hours after initiating step (c).

In some aspects, the methods disclosed herein further comprise (d) replacing a portion of the culture medium with a fresh culture medium comprising at least one expanding agent approximately 23 hours after initiating step (c).

In some aspects, the methods disclosed herein further comprise (d) replacing a portion of the culture medium with a fresh culture medium comprising at least one expanding agent approximately 24 hours after initiating step (c).

In some aspects, the methods disclosed herein further comprise (e) passaging and culturing transfected cells in culture medium comprising at least one expanding agent approximately8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after initiating step (d).

In some aspects, the methods disclosed herein further comprise (e) passaging and culturing transfected cells in culture medium comprising at least one expanding agent approximately 72 hours after initiating step (d).

In some aspects, the methods disclosed herein further comprise (e) passaging and culturing transfected cells in culture medium comprising at least one expanding agent approximately 73 hours after initiating step (d).

In some aspects, the methods disclosed herein further comprise (e) passaging and culturing transfected cells in culture medium comprising at least one expanding agent approximately 74 hours after initiating step (d).

In some aspects, the methods disclosed herein further comprise (e) passaging and culturing transfected cells in culture medium comprising at least one expanding agent approximately 75 hours after initiating step (d).

In some aspects, the methods disclosed herein further comprise (e) passaging and culturing transfected cells in culture medium comprising at least one expanding agent approximately 76 hours after initiating step (d).

In some aspects, the methods disclosed herein further comprise (e) passaging and culturing transfected cells in culture medium comprising at least one expanding agent approximately 77 hours after initiating step (d).

In some aspects, the methods disclosed herein further comprise (e) passaging and culturing transfected cells in culture medium comprising at least one expanding agent approximately 78 hours after initiating step (d).

In some aspects, the methods disclosed herein further comprise (e) passaging and culturing transfected cells in culture medium comprising at least one expanding agent approximately 79 hours after initiating step (d).

In some aspects, the methods disclosed herein further comprise (e) passaging and culturing transfected cells in culture medium comprising at least one expanding agent approximately 80 hours after initiating step (d).

In some aspects, the methods disclosed herein further comprise (e) passaging and culturing transfected cells in culture medium comprising at least one expanding agent approximately 81 hours after initiating step (d).

In some aspects, the methods disclosed herein further comprise (e) passaging and culturing transfected cells in culture medium comprising at least one expanding agent approximately 82 hours after initiating step (d).

In some aspects, the methods disclosed herein further comprise (e) passaging and culturing transfected cells in culture medium comprising at least one expanding agent approximately 83 hours after initiating step (d).

In some aspects, the methods disclosed herein further comprise (e) passaging and culturing transfected cells in culture medium comprising at least one expanding agent approximately 84 hours after initiating step (d).

In some aspects, the methods disclosed herein further comprise (e) passaging and culturing transfected cells in culture medium comprising at least one expanding agent approximately 85 hours after initiating step (d).

In some aspects, the methods disclosed herein further comprise (e) passaging and culturing transfected cells in culture medium comprising at least one expanding agent approximately 86 hours after initiating step (d).

In some aspects, the methods disclosed herein further comprise (e) passaging and culturing transfected cells in culture medium comprising at least one expanding agent approximately 87 hours after initiating step (d).

In some aspects, the methods disclosed herein further comprise (e) passaging and culturing transfected cells in culture medium comprising at least one expanding agent approximately 88 hours after initiating step (d).

In some aspects, the methods disclosed herein further comprise (e) passaging and culturing transfected cells in culture medium comprising at least one expanding agent approximately 89 hours after initiating step (d).

In some aspects, the methods disclosed herein further comprise (e) passaging and culturing transfected cells in culture medium comprising at least one expanding agent approximately 90 hours after initiating step (d).

In some aspects, the methods disclosed herein further comprise (e) passaging and culturing transfected cells in culture medium comprising at least one expanding agent approximately 91 hours after initiating step (d).

In some aspects, the methods disclosed herein further comprise (e) passaging and culturing transfected cells in culture medium comprising at least one expanding agent approximately 92 hours after initiating step (d).

In some aspects, the methods disclosed herein further comprise (e) passaging and culturing transfected cells in culture medium comprising at least one expanding agent approximately 93 hours after initiating step (d).

In some aspects, the methods disclosed herein further comprise (e) passaging and culturing transfected cells in culture medium comprising at least one expanding agent approximately 94 hours after initiating step (d).

In some aspects, the methods disclosed herein further comprise (e) passaging and culturing transfected cells in culture medium comprising at least one expanding agent approximately 95 hours after initiating step (d).

In some aspects, the methods disclosed herein further comprise (e) passaging and culturing transfected cells in culture medium comprising at least one expanding agent approximately 96 hours after initiating step (d).

In some aspects, after step (e) the transfected cells in culture medium are transferred to a cell culture bag, a cell culture flask, or a culture device.

In some aspects, the transfected cells in culture medium are transferred to the cell culture bag.

In some aspects, the culture device is a bioreactor.

In some aspects, the transfected cells are expanded by culturing the transfected cells in culture medium comprising at least one expanding agent about every 2 to 3 days or about every 3 to 4 days until about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, or about 28 days after initiating step (a).

In some aspects, the transfected cells are expanded until about 10 days after initiating step (a).

In some aspects, the transfected cells are expanded until about 14 days after initiating step (a).

In some aspects, the transfected cells are expanded until about 17 days after initiating step (a).

In some aspects, the transfected cells are expanded until about 21 days after initiating step (a).

In some aspects, the transfected cells are expanded until about 28 days after initiating step (a).

In some aspects, the methods disclosed herein further comprise (f) isolating the cells from the cell culture to obtain a cell population comprising the genetically modified CIK cells.

In some aspects, the transfected cells are cultured to obtain a cell population comprising the genetically modified CIK cells.

In some aspects, the methods disclosed herein further comprise the step of freezing the genetically modified CIK cells.

### III. Cells

In some aspects, the genetically modified CIK cells disclosed herein express one or more T cell receptors (TCR-CIK), chimeric antigen receptors (CAR-CIK), a genetically modified adhesion molecule, a genetically modified costimulatory ligand, a genetically modified cytokine receptor, a genetically modified chemokine receptor, a genetically modified cytokine, a genetically modified chemokine, an enzyme, a secreted drug, a checkpoint inhibitor, or a ligand.

In some aspects, the genetically modified CIK cells express a prodrug converting enzyme, an IgG-degrading enzyme of S. pyogenes (IdeS), a consensus variant 1 (CV1) protein, Intercellular Adhesion Molecule 1 (ICAM-1), CD137L, OX40L, CD70, IL-15 receptor, CCR5, CCR4, CD25, CD122, CD132, C-X-C chemokine receptor type 4 (CXCR4), IL-15, IL-18, IL-21 IL-23 IL-33, and IL-1a, IL-1b, matrix metalloproteinase (MMP), an enzyme for extracellular matrix (ECM) degradation, heparinase, an anti-PD-1 antibody or fragment thereof, an anti-T cell immunoglobulin and mucin-domain containing-3 (TIM-3) antibody or fragment thereof, IL-3 zetakine, alloimmune defense receptor (ADR)-41BBL, or any combination thereof.

### Chimeric Antigen Receptor (CAR)

In some aspects, the cell, e.g., the genetically modified CIK cell, comprises a CAR (e.g., a CAR-CIK cell).

In some aspects, the CAR is designed as a standard CAR, a split CAR, an off-switch CAR, an on-switch CAR, a first-generation CAR, a second-generation CAR, a third-generation CAR, or a fourth-generation CAR. In some aspects, the CAR comprises antigen-binding domain, a transmembrane domain, a costimulatory domain, an intracellular signaling domain, or any combinations thereof.

In some aspects, the CAR specifically binds (i.e., target) one or more antigens expressed on a tumor cell, such as a malignant B cell, a malignant T cell, or a malignant plasma cell.

In some aspects, the the chimeric antigen receptor (CAR) is specific for CD19, CD123, TIM-3, C-type lectin-like molecule-1 (CLL-1), CD70, mucin 1 (MUC-1), CD20, CD22, B-cell activating factor receptor (BAFFR), CD23, cytokine receptor like factor 2 (CRLF2), CD79b, CD79d, CD7, CD43, CD5, CD25, Lewis Y (LeY), natural killer group 2 member D (NKG2D), receptor tyrosine kinase like orphan receptor 1 (ROR1), receptor tyrosine kinase like orphan receptor 2 (ROR2), Wilms' tumor 1 (WT1), CD44 variant 6 (CD44v6), CD33, CD3ε, human epidermal growth factor receptor 2 (Her2), epidermal growth factor receptor (EGFR), epidermal growth factor receptor variant III (EGFRvIII), CA125, CD138, prostate-specific membrane antigen (PSMA), B7 homolog 3 protein (B7-H3), CD30, disialoganglioside GD-2, disialoganglioside GD-3, CD171, mesothelin (MSLN), ephrin type-A receptor 2 (EphA2), carcinoembryonic antigen (CEA), vascular endothelial growth factor receptor (VEGFR), IL13Rα2, prostate stem cell antigen (PSCA), epithelial cellular adhesion molecule (EpCAM), chondroitin sulfate proteoglycan 4 (CSPG4), folate receptor alpha (FRα), fibroblast activation protein (FAP), carbonic anhydrase IX (CAIX), B-cell maturation antigen (BCMA), signaling lymphocytic activation molecule family member 7 (SLAM7), CD126, mesenchymal-epithelial transition factor (c-MET), AXL receptor tyrosine kinase (AXL), CD133, tumor endothelial marker 8 (T_{EM}8), tumor-associated calcium signal transducer 2 (TROP2), programmed death-ligand 1 (PD-L1), delta-like ligand 3 (DLL3), melanoma-associated antigen 1 (MAGE-1), protein tyrosine kinase 7 (PTK7), c-type lectin domain containing 14A (CLEC14A), C-X-C chemokine receptor type 4 (CXCR4), New York esophageal squamous cell carcinoma 1 (NY-ESO-1) CD126, tumor-associated glycoprotein 72 (TAG-72), Guanylate cyclase 2C (GUCY2C), Cadherin-6 (CDH6), cadherin 17 (CDH17), claudin18.2 (CLDN18.2), glypican-3 (GPC3), CD147, CD16, glycoprotein 100 (gp100)/human leukocyte antigen-A2 (HLA-A2) complex, MACEP, urokinase-type plasminogen activator receptor (uPAR), alkaline phosphatase, placental-like 2 (ALPPL2), CD47, olfactory receptor 2H1 (OR2H1), matrix metalloproteinase-2 (MMP-2), podoplanin (PDPN), GDNF family receptor alpha-4 (GFRα4), thyrotropin receptor (TSHR), L1 cell adhesion molecule (L1-CAM), or any combination thereof.

In some aspects, the chimeric antigen receptor is specific for CD19.

In some aspects, the costimulatory domain comprises a costimulatory domain of an interleukin-2 receptor (IL-2R), interleukin-12 receptor (IL-12R), IL-7, IL-21, IL-23, IL-15, CD2, CD3, CD4, CD7, CD8, CD27, CD28, CD30, CD40, 4-1BB/CD137, ICOS, lymphocyte function-associated antigen-1 (LFA-1), LIGHT, NKG2C, OX40, DAP10, or any combination thereof. In some aspects, the costimulatory domain comprises a 4-1BB/CD137 costimulatory domain.

In some aspects, the transmembrane domain comprises a transmembrane domain of KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, CD19, or any combination thereof. In some aspects, the transmembrane domain comprises a CD28 transmembrane domain.

In some aspects, the intracellular signaling domain comprises an intracellular signaling domain derived from CD3 zeta, FcR gamma, common FcR gamma (FCER1G), Fc gamma RIIa, FcR beta (Fc Epsilon Rib), CD3 gamma, CD3 delta, CD3 epsilon, CD22, CD79a, CD79b, CD278 (“ICOS”), FcERI, CD66d, CD32, DAP10, DAP12, or any combination thereof. In some aspects, the intracellular signaling domain comprises a CD3 zeta intracellular signaling domain.

### T Cell Receptor-Engineered (TCR) Cells

In some aspects, an immune cell, e.g., the genetically modified CIK cell, disclosed herein comprises a T cell receptor (TCR), e.g., an engineered TCR. In some aspects, the TCR specifically binds to a tumor antigen. As used herein, the term “engineered TCR” or “engineered T-cell receptor” refers to a T-cell receptor (TCR) engineered to specifically bind with a desired affinity to HLA/peptide target antigen that is selected, cloned, and/or subsequently introduced into a population of immune cells, e.g., T cells, NK cells, and/or TILs.

In some aspects, the TCR specifically binds (i.e., targets) one or more antigens expressed on a tumor cell, such as a malignant B cell, a malignant T cell, or a malignant plasma cell. In some aspects, the TCR specifically binds a tumor antigen/HLA complex. In some aspects, the tumor antigen is, or is derived from, AFP, CD19, BCMA, CLL-1, CS1, CD38, CD19, TSHR, CD123, CD22, CD30, CD171, CD33, EGFRVIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-11Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E₂, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIRI, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, neoantigen, or any combinations thereof.

In certain aspects, an engineered cell of the present disclosure can express a T cell receptor (TCR) targeting an antigen. In some aspects, the TCR engineered cells can target main types: shared tumor-associated antigens (shared TAAs) and unique tumor-associated antigens (unique TAAs), or tumor-specific antigens. The former can include, without any limitation, cancer-testis (CT) antigens, overexpressed antigens, and differentiation antigens, while the latter can include, without any limitation, neoantigens and oncoviral antigens. Human papillomavirus (HPV) E6 protein and HPV E7 protein belong to the category of oncoviral antigens.

In some aspects, the TCR engineered cells can target a CT antigen, e.g., melanoma-associated antigen (MAGE) including, but not limited to, MAGE-A1, MAGE-A2, MAGE-A3, MAGE-A4, MAGE-A6, MAGE-A8, MAGE-A9.23, MAGE-A10, and MAGE-A12. In some aspects, the TCR engineered cells can target glycoprotein (gp100), melanoma antigen recognized by T cells (MART-1), and/or tyrosinase, which are mainly found in melanomas and normal melanocytes. In some aspects, the TCR engineered cells can target Wilms tumor 1 (WT1), i.e., one kind of overexpressed antigen that is highly expressed in most acute myeloid leukemia (AML), acute lymphoid leukemia, almost every type of solid tumor and several critical tissues, such as heart tissues. In some aspects, the TCR engineered cells can target mesothelin, another kind of overexpressed antigen that is highly expressed in mesothelioma but is also present on mesothelial cells of several tissues, including trachea.

In some aspects, the TCR engineered cells can target any neoantigen, which can be formed by random somatic mutations specific to individual tumors. In some aspects, the TCR specifically binds to (i.e., targets) a cancer antigen selected from the group consisting of AFP, Braf, CD19, TRAC, TCRβ, BCMA, CLL-1, CS1, CD3ε, CD19, TSHR, CD123, CD22, CD30, CD171, CD33, EGFRVIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-1 IRa, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRCSD, CXORF61, CD97, CD179a, ALK, Polysialic acid, PLAC1, GloboH, NY-BR-1, UPK2, HAVCR, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYPIB1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIRI, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, or any combinations thereof.

In certain aspects, the TCR specifically binds (i.e., targets) hTERT. In some aspects, the TCR specifically binds (i.e., targets) KRAS. In some aspects, the TCR specifically binds (i.e., targets) Braf. In some aspects, the TCR specifically binds (i.e., targets) TGFBRII. In some aspects, the TCR specifically binds (i.e., targets) MAGE A10/A4. In some aspects, the TCR specifically binds (i.e., targets) AFP. In some aspects, the TCR specifically binds (i.e., targets) PRAME. In some aspects, the TCR specifically binds (i.e., targets) MAGE A1. In some aspects, the TCR specifically binds (i.e., targets) WT-1. In some aspects, the TCR specifically binds (i.e., targets) NY-ESO. In some aspects, the TCR specifically binds (i.e., targets) PRAME. In some aspects, the TCR specifically binds (i.e., targets) NY-ESO. In some aspects, the TCR specifically binds (i.e., targets) CD19.

In some aspects, the TCR comprises an intracellular gamma/delta domain. In some aspects, the TCR is an antibody-T-cell receptor (AbTCR) (see, e.g., Xu et al., Cell Discovery 4:62 (2018), which is incorporated by reference herein in its entirety.

### IV. Pharmaceutical Compositions and Formulations

Pharmaceutical compositions suitable for administration to human patients are typically formulated for parenteral administration, e.g., in a liquid carrier, or suitable for reconstitution into liquid solution or suspension for intravenous administration.

In general, such compositions typically comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions can be employed as carriers, particularly for injectable solutions. Liquid compositions for parenteral administration can be formulated for administration by injection or continuous infusion. Routes of administration by injection or infusion include intravenous, intraperitoneal, intramuscular, intrathecal and subcutaneous.

In some aspects, the genetically modified cytokine induced killer (CIK) cells obtained by the methods disclosed herein are present in a pharmaceutical composition. As such, some aspects of the present disclosure are directed to a pharmaceutical composition comprising the genetically modified CIK cells obtained by the methods disclosed herein with a pharmaceutically acceptable carrier, diluent, solubilizer, emulsifier, preservative and/or adjuvant.

In some aspects, acceptable formulation materials preferably are nontoxic to recipients at the dosages and concentrations employed. In some aspects, the formulation material(s) are for s.c. and/or I.V. administration. In some aspects, the pharmaceutical composition comprises formulation materials for modifying, maintaining or preserving, for example, the pH, osmolality, viscosity, clarity, color, isotonicity, odor, sterility, stability, rate of dissolution or release, adsorption or penetration of the composition. In some aspects, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995).

In some aspects, the optimal pharmaceutical composition will be determined by one skilled in the art depending upon, for example, the intended route of administration, delivery format and desired dosage. See, for example, Remington's Pharmaceutical Sciences, supra.

In some aspects, the primary vehicle or carrier in a pharmaceutical composition is either aqueous or non-aqueous in nature. For example, in some aspects, a suitable vehicle or carrier is water for injection, physiological saline solution or artificial cerebrospinal fluid, possibly supplemented with other materials common in compositions for parenteral administration. In some aspects, the saline comprises isotonic phosphate-buffered saline. In some aspects, neutral buffered saline or saline mixed with serum albumin are further exemplary vehicles. In some aspects, pharmaceutical compositions comprise Tris buffer of about pH 7.0-8.5, or acetate buffer of about pH 4.0-5.5. In some aspects, the pharmaceutical composition further comprises sorbitol or a suitable substitute therefore. In some aspects, a composition comprising the genetically modified CIK cells is prepared for storage by mixing the selected composition having the desired degree of purity with optional formulation agents (Remington's Pharmaceutical Sciences, supra) in the form of a lyophilized cake or an aqueous solution.

In some aspects, the pharmaceutical composition is selected for parenteral delivery.

In some aspects, the formulation components are present in concentrations that are acceptable to the site of administration. In some aspects, buffers are used to maintain the composition at physiological pH or at a slightly lower pH, typically within a pH range of from about 5 to about 8.

In some aspects, when parenteral administration is contemplated, a therapeutic composition is in the form of a pyrogen-free, parenterally acceptable aqueous solution comprising the genetically modified CIK cells obtained by the methods of the present disclosure, in a pharmaceutically acceptable vehicle. In some aspects, a vehicle for parenteral injection is sterile distilled water in which the genetically modified CIK cells obtained by the methods disclosed herein formulated as a sterile, isotonic solution, and properly preserved. In some aspects, the preparation involves the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In some aspects, hyaluronic acid is also used. Hyaluronic acid, when present, can have the effect of promoting sustained duration in the circulation. In some aspects, implantable drug delivery devices are used to introduce the desired molecule.

In some aspects, a pharmaceutical composition involves the genetically modified CIK cells obtained by the methods of the present disclosure in a mixture with non-toxic excipients which are suitable for the manufacture of tablets. In some aspects, by dissolving the tablets in sterile water, or another appropriate vehicle, solutions are prepared in unit-dose form. In some aspects, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving the genetically modified CIK cells obtained by the methods of the present disclosure in sustained- or controlled-delivery formulations. In some aspects, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In some aspects, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly(2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra) or poly-D(−)-3-hydroxybutyric acid (EP 133,988). In some aspects, sustained release compositions can also include liposomes, which can be prepared by any of several methods known in the art. See, e.g., Eppstein et al, Proc. Natl. Acad. Sci. USA, 82:3688-3692 (1985); EP 036,676; EP 088,046 and EP 143,949.

The pharmaceutical composition to be used for in vivo administration typically is sterile. In some aspects, this is accomplished by filtration through sterile filtration membranes. In some aspects, where the composition is lyophilized, sterilization using this method is conducted either prior to or following lyophilization and reconstitution. In some aspects, the composition for parenteral administration is stored in lyophilized form or in a solution. In some aspects, parenteral compositions generally are placed into a container having a sterile access port, for example, an intravenous solution bag or vial having a stopper pierceable by a hypodermic injection needle.

In some aspects, once the pharmaceutical composition has been formulated, it is stored in sterile vials as a solution, suspension, gel, emulsion, solid, or as a dehydrated or lyophilized powder. In some aspects, such formulations are stored either in a ready-to-use form or in a form (e.g., lyophilized) that is reconstituted prior to administration.

### V. Methods of Treatment

Some aspects of the present disclosure are directed to methods of treating a subject in need thereof comprising administering to the subject a population of the genetically modified CIK cells obtained by the methods of the present disclosure.

In some aspects, the genetically modified CIK cells obtained by the methods of the present disclosure or the compositions as disclosed herein, for use in the prevention or treatment of cancers, tumors, viral infections, inflammatory diseases and disorders, autoimmune diseases and disorders, or any combination thereof.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986);); Crooks, Antisense drug Technology: Principles, strategies and applications, 2^{nd} Ed. CRC Press (2007) and in Ausubel et al. (1989) Current Protocols in Molecular Biology (John Wiley and Sons, Baltimore, Md.).

The following examples are offered by way of illustration and not by way of limitation.

### EXAMPLES

### Example 1

### Expansion of CAR-CIK Cells in Culture

CAR-CIK cells were prepared by a continuous process using fresh donor-derived whole blood as the starting material without requiring irradiated or non-irradiated feeder cells. See e.g., Table 2 below. The manufacturing process can be divided into four distinct steps: donor peripheral blood mononuclear cells (PBMCs) isolation, culture and differentiation, committed CIK precursor cell stimulation and expansion, committed CIK precursor cell electroporation, and expansion of electroporated, committed precursor cells into CAR-CIK cells.

### Donor PBMC Isolation, Culture and Differentiation

On Day 0, a healthy donor whole blood was diluted 1:1 with phosphate buffered saline (PBS), layered over Histopaque®-1077 gradient (MilliporeSigma) and centrifuged. The PBMCs layer was collected and pooled. PBMCs were washed with PBS and resuspended in Advanced RPMI 1640 media supplemented with 10% heat-inactivated Fetal Bovine Serum (FBS), 2 mM L-glutamine and 1,000 IU/mL INF gamma-1b (Actimmune®) at 37±1 °C and 5±1% CO₂ at density of 3×10\hat{ }6 cells/mL.

### Committed CIK Precursor Cell Stimulation and Expansion

The next day (day 1), cells were stimulated with 50 ng/ml GMP grade anti-CD3 monoclonal antibody (Clone OKT3; Takara Bio) and 300 IU/mL recombinant human IL-2 (Proleukin®). On Day 2, after overnight incubation with OKT3 and IL-2, the resulting Committed CIK precursor cells were collected by centrifugation, washed with Phosphate Buffered Saline (PBS) to prepare for electroporation.

### Committed CIK Precursor Cell Stimulation and Expansion

Committed CIK precursor cells were resuspended at a concentration of 50 million cells/ml in electroporation medium (CoStorSol®; Preservation Solutions, Inc, Elkhorn, WI, USA) for non-viral genetic modification. CoStorSol® is a sterile, non-pyrogenic solution for hypothermic flushing and storage of organs and is used to cool cellular material and lower its metabolic requirements during the electroporation step. Thirty million cells in 0.6 mL suspension were added to each electroporation cuvette (gap size 4 mm) for processing with the Gene Pulser Xcell™ device (BioRad). Each cuvette contained premixed 0.2 μg of SB100X RNA and 1.5 μg of 3^{rd} generation pT4-CD19-CAR plasmid per million cells. Each cuvette was pulsed at ambient temperature with the defined Square Wave program listed in Table 1 below. Program settings were verified prior to proceeding with the electroporation. Electroporators were calibrated on a routine schedule.

TABLE 1
Parameters for Square Wave Electroporation
in Gene Pulser Xcell ™ Device

| Parameter | Setting | |
|---|---|---|
| Voltage | 500 | volts |
| Pulse Length | 5 | milliseconds |
| Pulse Number | 2 | pulses |
| Pulse Interval | 0.1 | second |

The electroporation step was monitored by Voltage and Droop readings on the device for each cuvette.

Immediately following electroporation, cells were transferred from the cuvette to a T25 cell culture flask containing 12 mL pre-warmed Advanced RPMI+20% FBS+2 mM L-glutamine for a seeding density of 2.5×10\hat{ }5 cells/ml and allowed to recover for 12-18 hrs in an incubator at 37.0±1.0 °C, 5.0±1.0% CO₂. Each cuvette corresponds to one T25 flask.

The following day (Day 3), one half of the medium was replaced with an equal volume of Advanced RMPI supplemented with 10% FBS, 2 mM L-glutamine, and 600 IU/mL IL-2 (Proleukin®) (for final culture concentration of 300 IU/mL). Cells were incubated for a further 96 hours (4 days).

Expansion of Electroporated, Committed Precursor Cells into CAR-CIK Cells

On Day 7 the cells were split for in either tissue culture flasks, gas-permeable cell culture bags, or Gas Permeable Rapid Expansion (G-Rex®) closed-system bioreactor (Wilson Wolf Manufacturing, New Brighton, MN).

Cells cultured in flasks (Nunc™ EasYFlask™) were seeded at a density of 7.5×10⁵ cells/mL in Advanced RMPI supplemented with 10% FBS, 2 mM L-glutamine and 300 IU/mL IL-2 whereas cells cultured in bags (Saint-Gobain VueLife™ C-Series, FEP) were seeded at a density of 5.0 to 5.2×10⁵ cells/mL in the same medium. Both sets of cells (cultured in flasks and cell culture bags) were counted and expanded in the same medium, at their respective seeding densities, every 3-4 days for 17 or 21 days total (since the process start) prior to harvest and characterization. Cells were harvested. The harvested cells were characterized side by side for fold-expansion, cell viability and immunophenotype (see Table 3 below).

Cells cultured in G-Rex were seeded at density of 0.5×10\hat{ }6 cells/cm² in Advanced RMPI supplemented with 10% FBS, 2 mM L-glutamine and 300 IU/mL IL-2. At day 14, 70% of medium was replaced. At day 17 (since the process start) cells were harvested by centrifugation and characterized.

TABLE 2
Expansion of CAR-CIK Cells in Culture in the Absence of Irradiated Feeder Cells

Day 0: Cell processing from PB and separation of mononuclear cells on on density gradient at 2400 rpm
for 10 minutes at 21 degrees C (Acceleration = 5, Deceleration = 2)
1 Wash with PBS followed by centrifugation at 2400 rpm for 10 minutes at 21 degrees C (Acceleration =
MAX, Deceleration = MAX)
1 Wash with PBS followed by centrifugation at 1400 rpm for 10 minutes at 21 degrees C (Acceleration =
MAX, Deceleration = MAX)
1 Wash with complete medium (RPMI-160 Advanced Medium + 10% FBS + 2 mM L-glutamine) followed
by sampling for cell count and centrifugation at 1400 rpm for 10 minutes at 21 degrees C
(Acceleration = MAX, Deceleration = MAX)
Day 0: Cell culture initiation with RPMI-1640 Advanced medium, 10% FBS at 3 million cells/mL and
treatment with 1,000 IU/mL IFN-gamma
Day +1: Stimulation with cytokines at the final concentration: OKT3 50 ng/ml and rhIL-2 300 U/ml.
*Day 2: Harvest cells by centrifugation at 200 × g for 10 minutes at 21 degrees C (Acceleration = MAX,
Deceleration = MAX)
1 wash with PBS followed by centrifugation at 200 × g for 10 minutes at 21 degrees C (Acceleration =
MAX, Deceleration = MAX)
Resuspend in CoStorSol to 50 million cells/ml (100 million cells/mL)
Electroporate 30 million cells (0.6 mL) (10 million cells, 0.1 mL) per cuvette with 0.2 μg of SB100X RNA
and 1.5 μg of pT4 transposon donor plasmid per million cells
Seed post-electroporation cells at 2.5 million cells/ml: 12 mL complete medium (RPMI-160 Advanced
Medium + 20% FBS + 2 mM L-glutamine) in a T25 cm² cell culture flask (4 mL in a single well of 6-well
plate)
Day +3: Change of half medium with complete medium supplemented with rhIL-2 at the final
concentration of 300 U/ml.
Day +7: Cell count, phenotypic analysis and transfer to cell culture bags or flasks. Cell expansion with
complete medium supplemented with rhIL-2 at the final concentration of 300 U/ml.
On day +10: +14: expand by diluting cells to 500,000 cells/mL in complete medium supplemented with
300 U/mL rhIL-2.
At the end of production (Day +17 or 21): cell count, phenotypic analysis, execution of functional tests
and freezing.
The electroporation information is for 4 mm cuvettes.
*In bold: conditions for a different size of cuvettes with 2 mm gap which can be used.

TABLE 3
Evaluation of cell phenotype in cultures expanded using flasks or culture bags*

| | | | | | | | | Day 21 |
|---|---|---|---|---|---|---|---|---|
| | | Seeding | | | | Day 21 | Day 21 | vector copy |
| | | Density | Overall | Day 21 | Day 21 | % | % | number |
| | Expansion | on day 7, | Fold | % | % | CD3+ | CD3+ | (VCN) |
| Donor | Vessel | Cells/mL | Expansion | Viability | CD3+ | CD56+ | CAR+ | cp/cell |
| 1 | Flasks | 7.5 × 10⁵ | 142 | 89 | 96.4 | 48.4 | 72.1 | 2.4 |
| | Bags | 5.0 × 10⁵ | 227 | 91 | 96.3 | 46.6 | 79.2 | 3.5 |
| 2 | Flasks | 7.5 × 10⁵ | 172 | 82 | 98.8 | 47.4 | 41.6 | Not |
| | | | | | | | | determined |
| | Bags | 5.15 × 10⁵ | 299 | 79 | 97.9 | 59.4 | 73.4 | Not |
| | | | | | | | | determined |

*CAR-CIK cells were engineered using the SB transposon system using 3^{rd} generation pT4-CD19CAR plasmid.

These results demonstrate that achieved phenotype of the cells as characterized by CD3, CD56, and CAR markers is comparable between experimental arms.

Electroporation on day 2, eliminated the need for the use of feeder cells (e.g., gamma irradiated or non-irradiated same donor PBMCs). The elimination of feeder cells (e.g., gamma irradiated or non-irradiated same donor PBMCs) is advantageous because, for example: (i) it is more practical for commercial production of CAR-CIKs (e.g., the need for irradiation increases the cost which will ultimately be reflected in the cost of a drug product); (ii) it eliminates the requirement for additional testing of e.g., feeder cells and raw material during production; and (iii) it decreases the risk of rejection of CAR-CIK cells (e.g., if there are irradiated feeders in the final product present they could prime host against CAR-CIK reaction).

Tables 4-8 show evaluation of large-scale manufacturing process for genetically modified CAR-CIK cells in bags.

At the end of this cellular production process, regardless of the harvest day, cells in the final preparation typify the CAR-CIK immuno-phenotype as indicated in Table 4. Summary data from 5 lots of CAR-CIK cells harvested on Day 17 and 5 lots harvested on Day 21 are presented.

TABLE 4
Phenotype of CAR-CIK Cells*

| | | Mean ± s.d. | Mean ± s.d. |
|---|---|---|---|
| Parameter | Target | Day 17 Harvest | Day 21 Harvest |
| % CD3⁺ | ≥90% | 98 ± 0.9% | 98 ± 1.5% |
| % CD56⁺ | ≥30% | 58 ± 14.3% | 68 ± 13.1% |
| (of % CD3⁺) | | | |
| % CAR⁺ | ≥20% | 68 ± 6.8% | 58 ± 16.6% |
| (of % CD3⁺) | | | |

*CAR-CIK cells were engineered using the SB transposon system using 3^{rd} generation pT4 CD19CAR plasmid.

As shown in Table 5, CAR-CIK cells exhibit the expected potency against target antigen-positive cancer cells in an in vitro cytotoxicity assay regardless of the day of harvest. Cytotoxicity of CAR-CIK cells (were engineered using the SB transposon system using 3^{rd} generation pT4 CD19CAR plasmid) was evaluated with a 4-hour co-culture assay. CARCIK-CD19 cells were co-cultured with CD19+ REH target cells at an effector to target ratio (E:T) of 5:1. Immediately prior to co-culture the REH cells were stained with 0.15 uM CFSE to distinguish them from CARCIK-CD19 cells. CFSE staining was quenched after 7 minutes by adding FBS and holding for 3 minutes. The REH cells were washed and resuspended in complete RPMI media at a final concentration of 4×10⁵ cells/mL. An equal volume of 2×10⁶ CARCIK-CD19 cells were added to the stained REH cells and incubated for 4 hours at 37 °C and 5% CO2. At the end of incubation, the cells were washed in PBS and resuspended in Annexin-V binding buffer and stained with Annexin V-EnzoGold for 15 minutes. Cells were immediately analyzed by flow cytometry by first gating on forward and side scatter which will include both CARCIK-CD19 and REH cells. Cells were further gated on CFSE positive cells to distinguish REH cells and then gated on Annexin+ cells. The percentage of cytotoxicity was calculated as the % of Annexin-V+ cells in the co-culture normalized for the % Annexin-V+ cells in the REH only well.

TABLE 5
Potency of CAR-CIK Cells* in vitro

| | | Mean ± s.d. | Mean ± s.d. |
|---|---|---|---|
| Parameter | Target | Day 17 Harvest | Day 21 Harvest |
| Cytotoxicity | ≥25% lysis of | 66 ± 21.3% | 40 ± 7.3% |
| | target cells¹ | | |

¹Lysis of CD19⁺ REH cells in a co-culture with CAR-CIK cells
*CAR-CIK cells were engineered using the SB transposon system using 3^{rd} generation pT4 CD19CAR plasmid.

The level of cellular expansion of the CAR-CIK cells over the production process was lower for lots harvested on Day 17 as compared to cells that underwent one additional passage for harvest on Day 21 as shown in Table 6.

TABLE 6
Fold Cellular Expansion from Day 2 to Harvest*

| | Mean ± s.d. | Mean ± s.d. |
|---|---|---|
| Parameter | Day 17 Harvest | Day 21 Harvest |
| Cellular Expansion | 45 ± 25.3-fold | 287 ± 137.1-fold |
| Day 2 to Harvest¹ | | |

¹Overall fold-expansion is the cumulative product of the fold-expansion at each passage relative to the number of cells electroporated on Day 2
*CAR-CIK cells were engineered using the SB transposon system using 3^{rd} generation pT4 CD19CAR plasmid.

The earlier harvest was also associated with a shift in the memory phenotype of the CAR-CIK cells toward a more naïve and central memory phenotype and away from the more differentiated effector phenotypes as shown in Table 7.

TABLE 7
Memory Phenotype of CAR-CIK Cells*

| | Mean ± s.d. | Mean ± s.d. |
|---|---|---|
| Parameter | Day 17 Harvest | Day 21 Harvest |
| Naive | 10.9 ± 7.23 | 2.2 ± 1.35 |
| CD3⁺/CAR⁺/CD45 RO⁻/CD62L⁺ | | |
| Central Memory | 44.9 ± 16.21 | 18.4 ± 11.67 |
| CD3⁺/CAR⁺/CD45 RO⁺/CD62L⁺ | | |
| Effector Memory | 38.1 ± 15.10 | 74.6 ± 9.54 |
| CD3⁺/CAR⁺/CD45 RO⁺/CD62L− | | |
| Effector | 6.1 ± 3.37 | 4.8 ± 2.51 |
| CD3⁺/CAR⁺/CD45 RO⁻/CD62L⁻ | | |

*CAR-CIK cells were engineered using the SB transposon system using 3^{rd} generation pT4 CD19CAR plasmid.

The earlier harvest of CAR-CIK cells on Day 17 still permitted a sufficient number of cell divisions to dilute away residual non-integrated CAR transgene-bearing plasmid such that the final product maintained a vector copy number below the widely accepted safety threshold of 5.0 as shown in Table 8. The labile nature of the RNA encoding the transposase source ensures a minimal risk of re-mobilization of the integrated CAR transgenes.

TABLE 8
Vector Copy Number Measurements from CAR-CIK Cells*

| | | Mean ± s.d. | Mean ± s.d. |
|---|---|---|---|
| Parameter | Target | Day 17 Harvest | Day 21 Harvest |
| Vector Copy Number | <5.0 cp/cell | 2.1 ± 0.66 | 1.4 ± 1.35 |

*CAR-CIK cells were engineered using the SB transposon system using 3^{rd} generation pT4 CD19CAR plasmid.

### Example 2

Comparison of Feeder Free CIK Process to the Process with Feeder Cells

CAR-CIK cells generation process utilizing feeder cells and electroporation on day 0 was compared to CAR-CIK cells generation feeder cells free process coupled with electroporation on day 2.

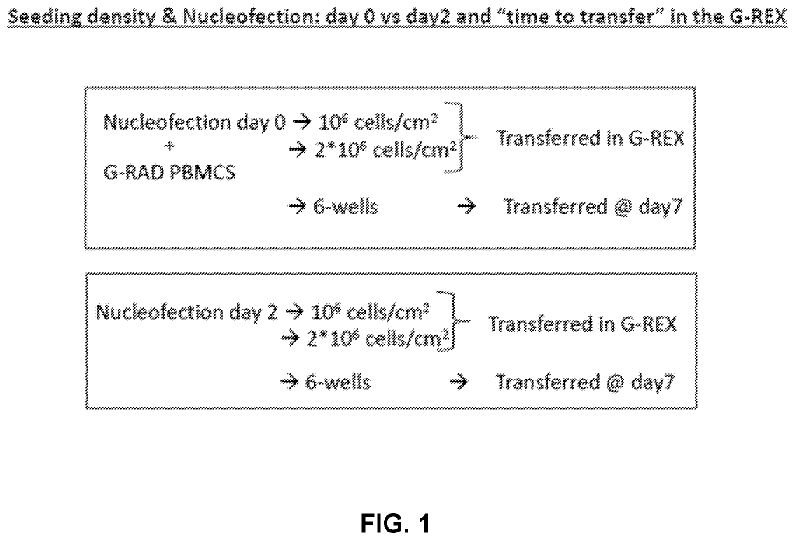

FIG. 1 shows schematic representation of experimental flow in experiments comparing feeder free CIK process to the original process with feeder cells. Both work flows utilize two seeding densities of cells transferred in the G-REX at day 0/day2: one million or two million of cells per square centimeter as indicated in the schema. Both work flows consider the additional variable of keeping the cells in the 6-wells after electroporation and then transferring the cells in the G-REX at day 7 (at a cell density of 10×⁶/cm²).

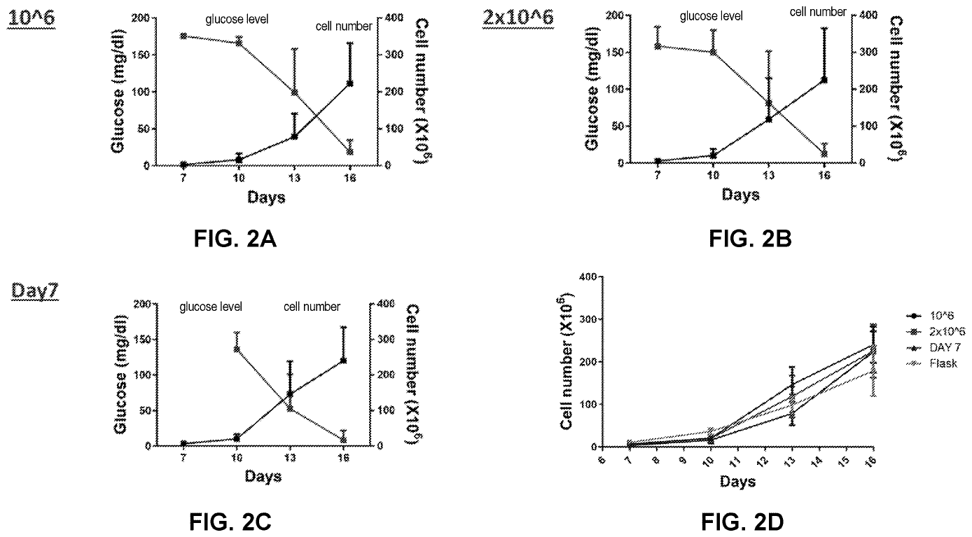

Cell expansions utilizing “the original CIK generation process” (Day 0 electroporation+γ irradiated feeder cells and expanded in flasks) were compared to “the Day 2 feeder free CIK generation process” (Day 2 electroporation without feeder cells; plating the cells immediately after electroporation at 1×10⁶ cells/cm² cell density (FIG. 2A) or 2×10⁶ cells/cm² cell (FIG. 2B) density; expanding the cells in the 6-well plates until day 7); transferring cells to G-Rex for expansion on Day 7 (FIG. 2C)). FIGS. 2A-2D demonstrate that cell expansion parameters measured such as cell numbers and level of glucose in the media are similar within the different culture conditions in feeder free, Day 2 electroporation process.

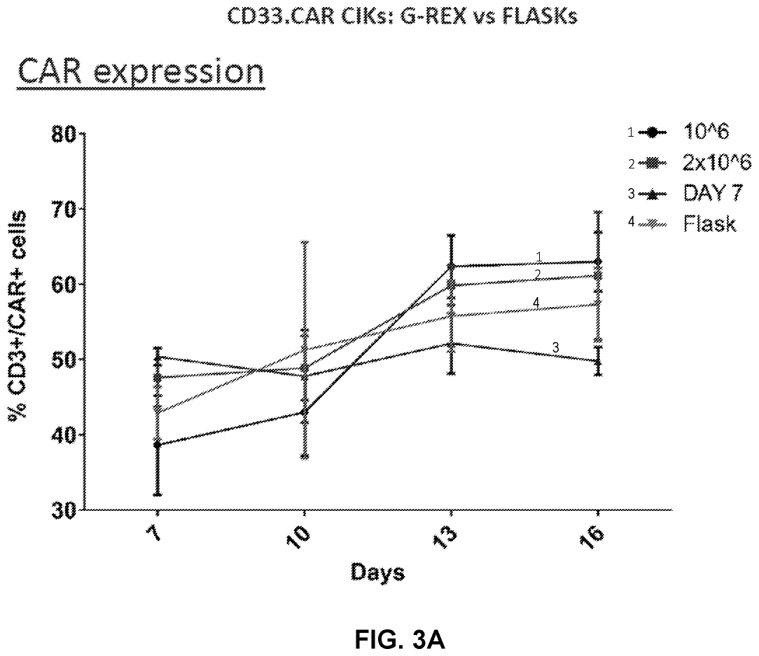

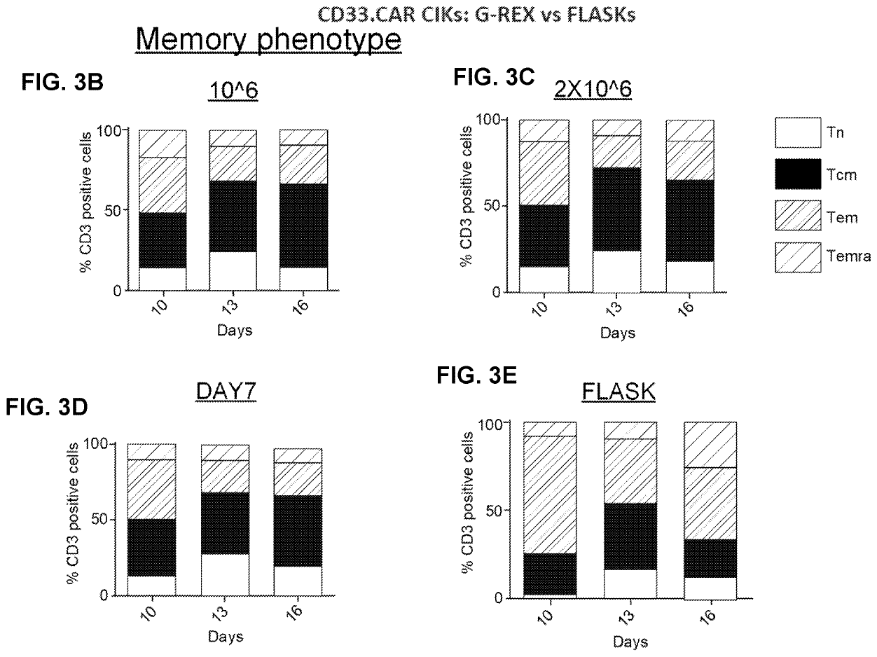

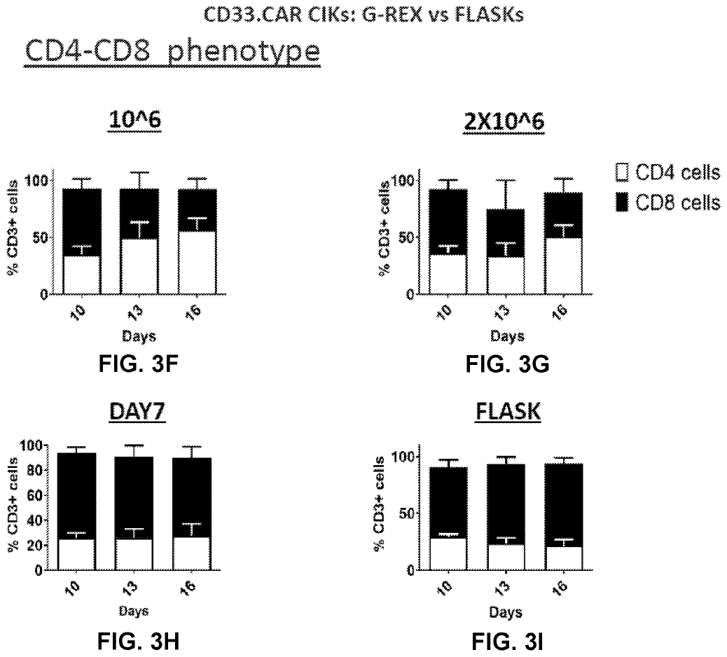

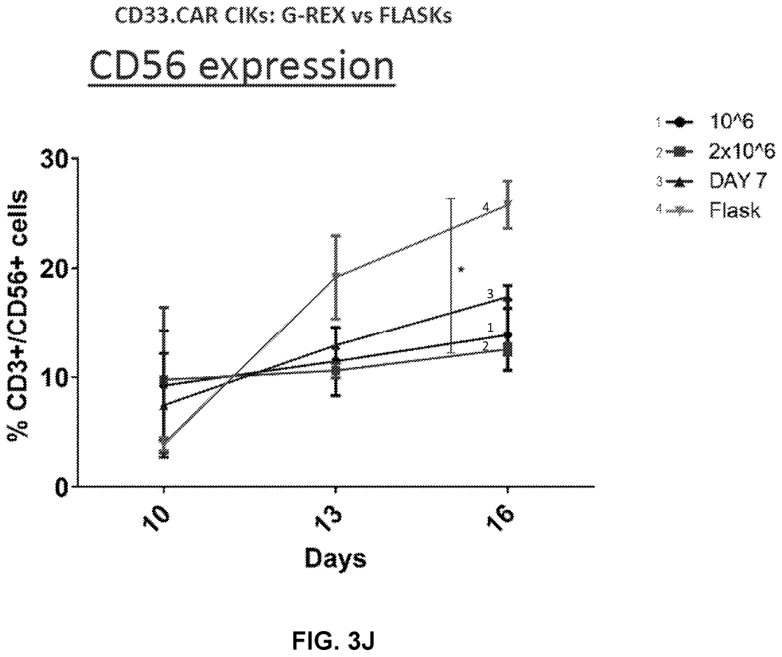

FIG. 3A shows that it is possible to obtain a high CAR expression (>50%) in all the conditions tested, similarly to the classical cell culture in flasks.

### Evolution of the Memory Phenotype

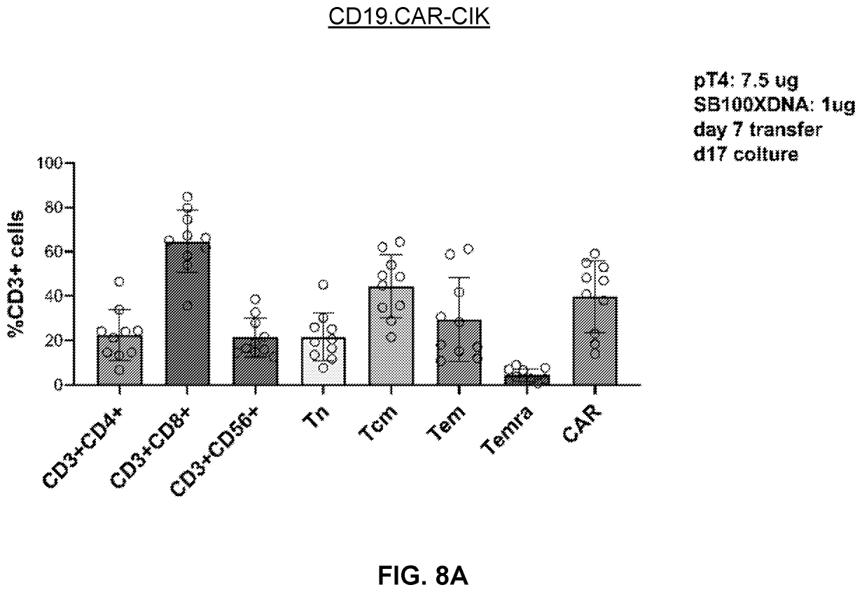

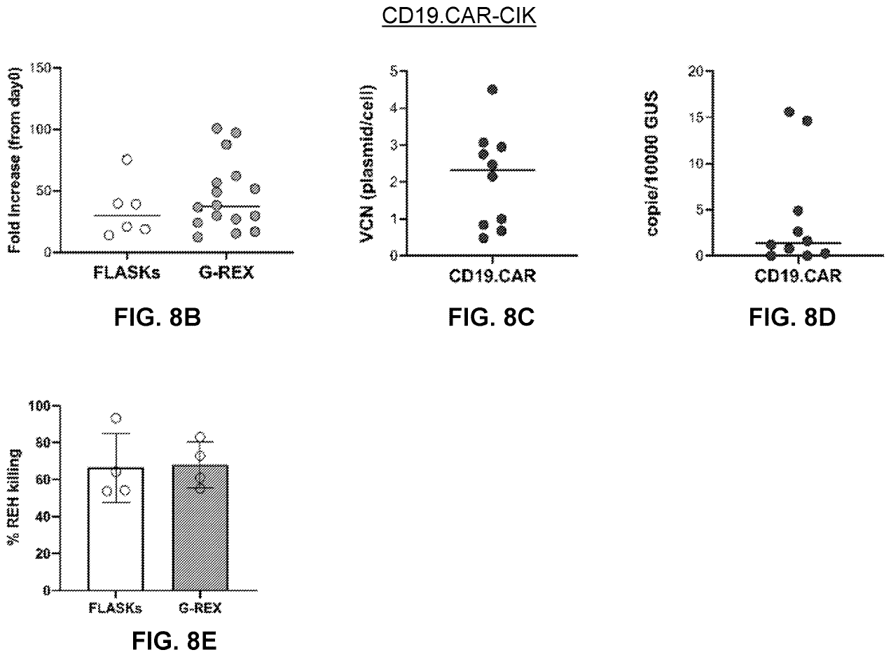

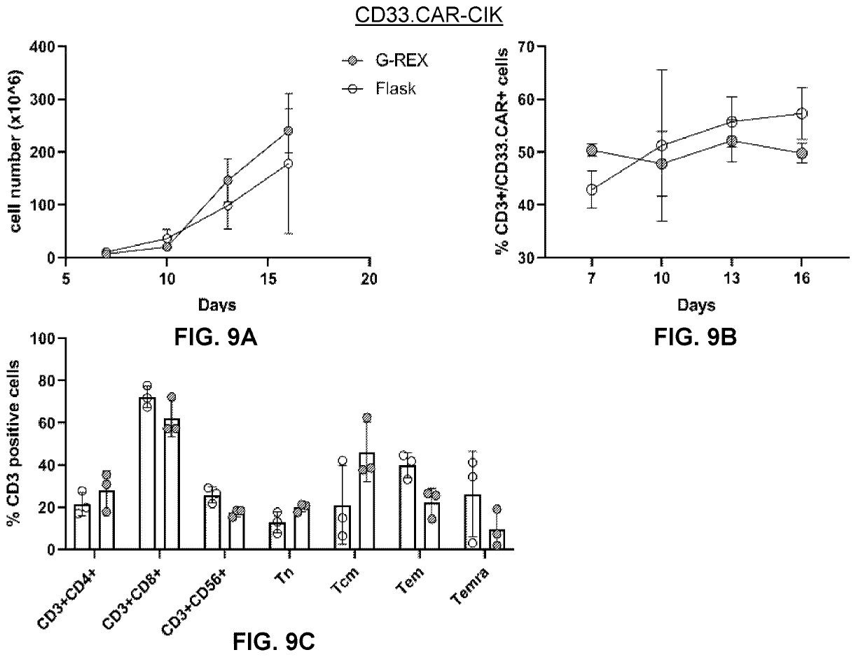

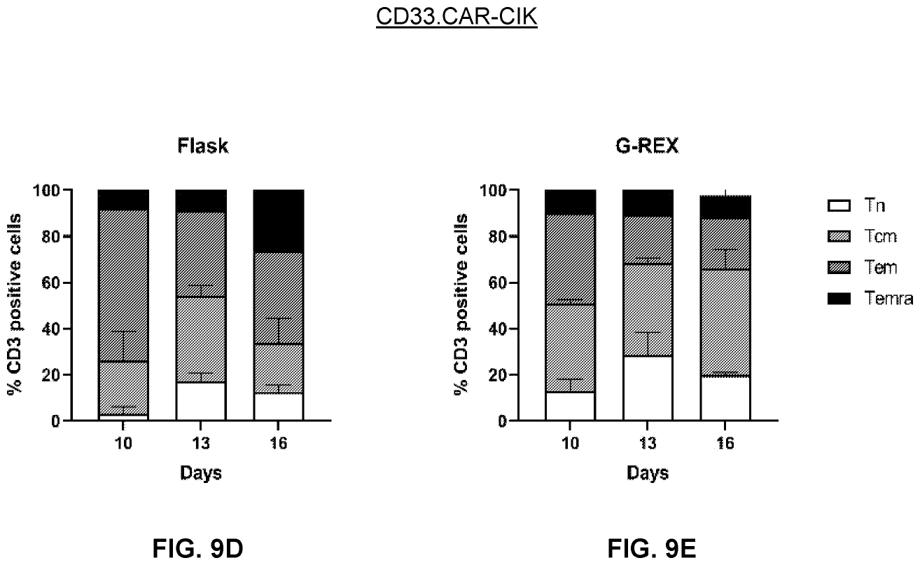

The comparison between the different conditions highlights how the Day 2 feeder free cell culture (CIK generation process: day 2 electroporation without feeder cells; plating the cells immediately after electroporation at 1×10⁶ cells/cm² cell density (FIG. 3B) or 2×10⁶ cells/cm² cell density (FIG. 3C); expanding the cells in the 6-well plates until day 7; transferring to G-Rex for expansion on day 7 (10×⁶ cells/cm²) (FIG. 3D) was able to better preserve a more immature memory phenotype of T central memory (Tcm) and T naïve (Tn) cells, as compared to the flask condition (FIG. 3E), which predominantly differentiate the cells in effector memory (Tem) and terminally differentiated Temra, the latter ones being associated to the lesser persistence in vivo. See also FIG. 8A and FIGS. 9D and 9E. These data show that the Day 2 feeder free CIK generation process enables production of healthy cells, with a good T cell fitness.

Displaying a more immature memory phenotype is recognized as one of the main variables implicated in the CAR T cell persistence in vivo. Therefore, enriching the memory compartments of Tem and In represents a further optimization of the CAR-CIK product.

### CD4-CD8 Phenotype Evolution

FIGS. 3F-3I show that in all the conditions tested both CD4+ and CD8+ cells were differentiated, with the Day 7 condition better mimicking the ratio of CD4:CD8 typical of CAR-CIK cells cultured in flasks.

### CD56 Phenotype Evolution

FIG. 3J shows that the CD56 expression in the G-Rex conditions is lower as compared to the flask. This is due to the fact that the cells are less manipulated in the bioreactor, so they are less activated and differentiated (as also observed by the memory phenotype).

### Short-Term Cytotoxicity Assay

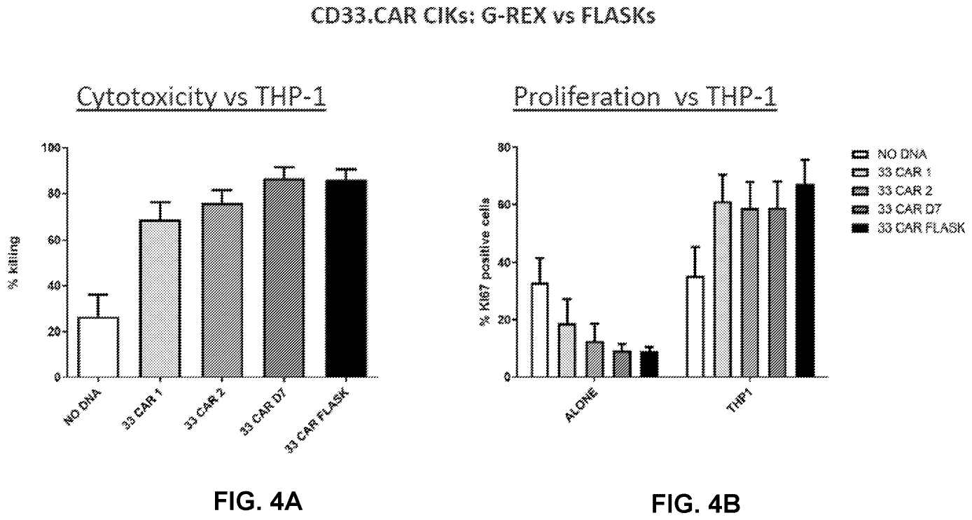

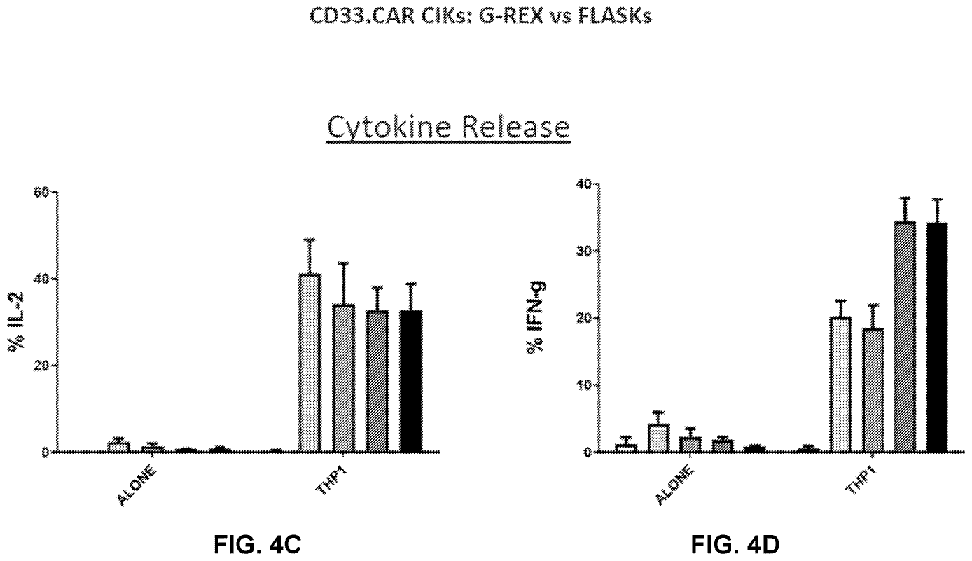

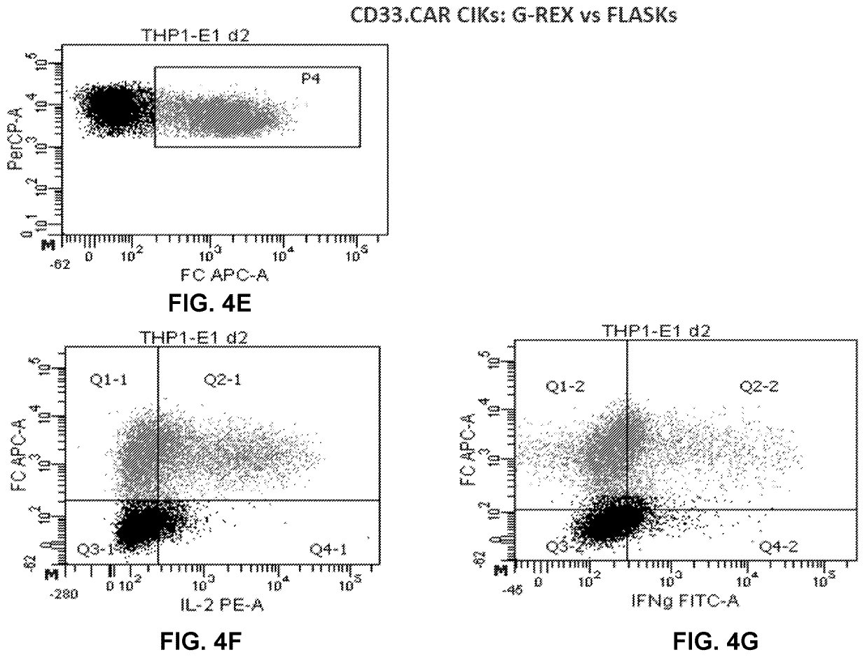

Target cells were labeled with PE-Cell Tracker. At the end of the incubation, target cell killing was measured through apoptosis detection by flow cytometry, after annexin V and 7-amino-actinomycin D (7-AAD) (AnnV-7AAD) staining, gating in the Cell Tracker PE+. The percentage of killed cells was calculated considering AnnV+ and AnnV+7AAD+ cells as compared to the basal positivity of AnnV and 7AAD of the target cells plated alone. FIG. 4A shows that CAR-CIK cells generated by the Day 2 feeder free method and expanded in G-REX have similar lytic activity to the CAR-CIK-CD33 cells generated using the original CIK generation process (day 0 electroporation+γ irradiated feeder cells and expanded in Flasks).

### Proliferation Assay

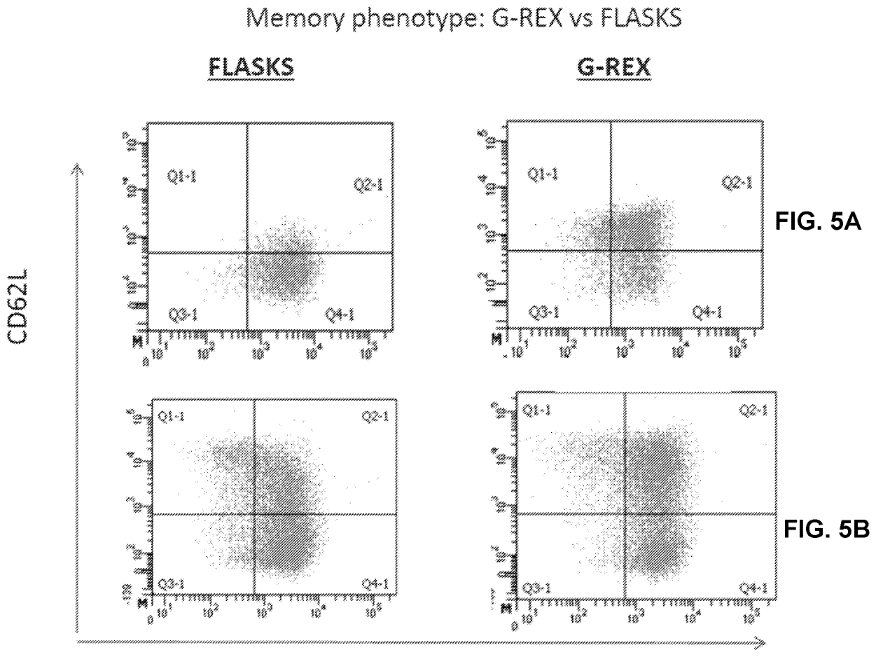

The proliferation ability of CAR-CIK cells was evaluated after co-culture with the Cell Tracker-labeled targets THP-1, irradiated at 100 Gy g-radiations at an E:T ratio of 1:1. After a 72 h co-culture, the cells were collected, immunostained for intracellular Ki-67, and then analyzed by flow cytometry by performing detection of Cell Tracker negative-Ki-67+ cells (therefore the proliferating CAR-CIK cells). FIG. 5B shows CAR-CIK cells generated by the Day2 feeder free method and expanded in G-REX have similar proliferative ability, when encountering the target cells, to the cells generated using the original CIK generation process (day 0 electroporation+γ irradiated feeder cells and expanded in Flasks).

### Cytokine Secretion Profile

Intracellular Cytokine Staining CAR-CIK cell ability to produce cytokines was evaluated following a stimulation with the target cell at an ET ratio of 1:3. After a 2 hr and 30 min co-culture, BD GolgiStop was added. The co-culture was then maintained for an additional period of 2 hr and 30 min, after which the cells were collected and stained for anti-CD3 and anti-Fc surface molecule (CAR) detection. Finally, intracellular cytokine staining (ICS) for IL-2 and IFN-γ was performed using the BD Cytofix/Cytoperm kit, according to the manufacturer's protocol. Specimens were then analyzed by flow cytometry. FIGS. 4E-4G show the cytokine production is CAR-specific in response to the target challenge.

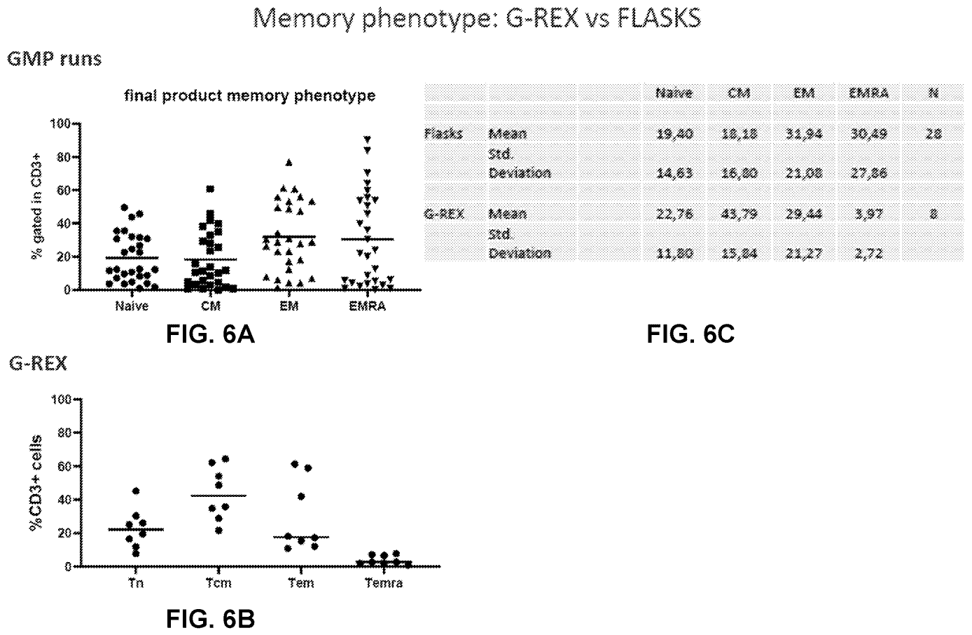

FIGS. 5A and 5B show the difference of the memory state between flasks and G-REX, starting from the same donor source (Donor A (FIG. 5A) and Donor B (FIG. 5B), suggesting that the G-REX device allows the cells to be more immature in their memory penotype. See also FIGS. 6A-6C.

### Day 0 Plasmid Titration

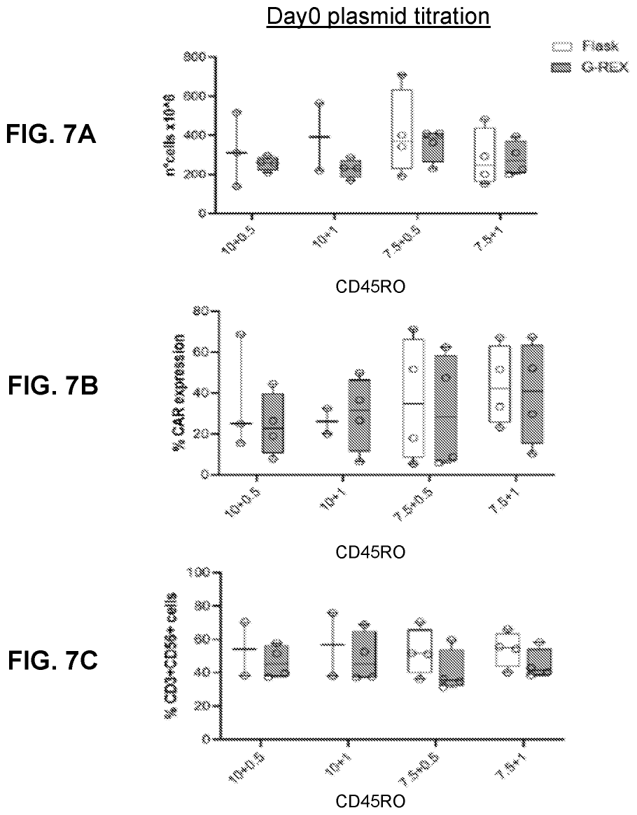

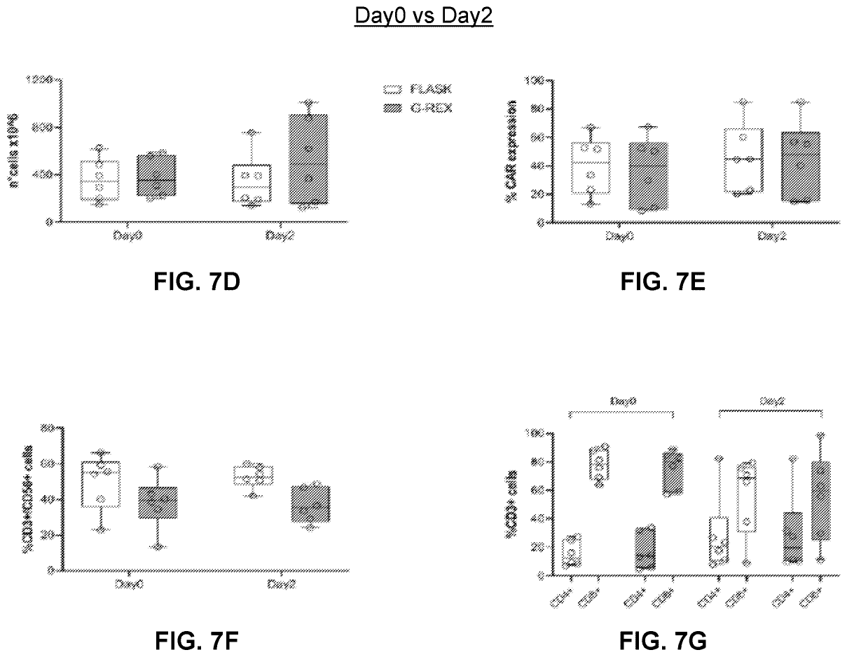

These experiments were done to titrate the novel SB molecules of pT4 transposon (carrying the CD19-CAR) and SB100×DNA. Different pT4: SB100X ratios were tested: 10+0.5, 10+1, 7.5+0.5, 7.5+1. FIGS. 7A-7C show that a good production of CAR-CIK cells was achieved in all the conditions, with the 7.5+1 as the preferred one, in terms of using less pT4 and thus total amount of DNA and in terms of cell numbers and CAR positivity.

FIGS. 7D-7G show Day 0 (+ feeder cells) and Day 2 (feeder free) comparison by using pT4 transposon and SB100×DNA (7.5+1). The Day 2 feeder free process ensures the production of high numbers of CAR-CIK cells carrying a high expression of the CAR, both in Flasks and G-REX, that is comparable to the standard Day 0 (+feeder cells) method.

Fold increase, vector copy number (VCN), SB100X detection, and in vitro cytotoxicity of CD19.CAR-CIK cells produced with the Day 2 feeder free method, with 7.5+1 SB plasmid ratio were tested.

### Fold Increase

The fold increase was calculated as the ratio between the number of cells expanded at the end of the culture and the number of cells stimulated at day 0.

### VCN Analysis

DNA was extracted from CD19.CAR-CIK cells using QIAamp DNA mini kit (QIAGEN, Hilden, Germany) and quantitative real-time PCR was performed on 100 ng of genomic DNA. Primers and probes were specific for the inverted repeat/direct repeat (IR/DR) sequences were as follows: forward primer 50-CTCGTTTTTCAACT ACTCCACAAATTTCT-30; reverse primer 50-GTGTCATGCAC AAAGTAGATGTCCTA-30; (FAM)-labeled probe 50-CTGACTT GCCAAAACT-30.

### Quantitative Real-Time PCR for Detection of Transposase

Enzyme RNA was extracted from CD19.CAR-CIK cells using the QIAamp RNA mini kit (QIAGEN) and retrotrascribed with SuperScript IV VILO master mix (Invitrogen). Amplification by qPCRs of homologous regions within the SB100X transcripts was performed using TaqMan gene expression master mix (Applied Biosystems, Thermo Fisher Scientific) and FastStart universal probe master (Roche Diagnostic, Mannheim, Germany). Primers were as follows: forward primer, 50-AAGCCGAAGAACACCATCC-30; reverse primer, 50-AGCACCCC CACAACATGA-30; UPL probe #87, 50 CTGACTTGCCAAAACT-30. The number of copies was quantified based on a seven-point standard curve of plasmidic DNA diltutions from 107 to 20 copies of transposase. b-glucuronidase gene (GUS) was used for data normalization by quantitative RT-PCR using the Ipsogen GUS control gene kit (QIAGEN), and analyzing data in agreement with the statistical delta-delta CT (ddCT) method.

### Short-Term Cytotoxicity Assays

In the short-term cytotoxic assay, CIK cells were co-cultured for 4 h with the CD19+ REH target cells (previously labeled with PE-cell tracker) at an E:T ratio of 5:1. At the end of the incubation, target cell killing was measured through apoptosis detection by flow cytometry, after annexin V and 7-amino-actinomycin D (7-AAD) (AnnV-7AAD) staining, gating in the Cell Tracker PE+ cells. The percentage of killed cells was calculated according to the following formula: (% annexin V+ target cells+% annexin V+7AAD+ target cells) after co-culture with CIK cells)−(% annexin V+ target cells+% annexin V+7AAD+) target cells alone/100−(% annexin V+ target cells+% annexin V+7AAD+ target cells alone).

FIGS. 8B-8E show that the Day 2 feeder free method grants the production of high numbers of CD19.CAR-CIK cells, carrying a high CAR expression, being compliant with the release criteria of VCN and SB100X transposase detection thresholds, and displaying a high cytotoxic activity against a CD19+ cell line.

FIGS. 9A-9C show that the day 2 feeder free method, either applied to the standard culturing using Flasks or using G-Rex device, grants high numbers of CAR+CIK cells, that display the typical phenotype of CIK cells in terms of CD4-CD8 ratio, CD56 positivity, and memory phenotype.

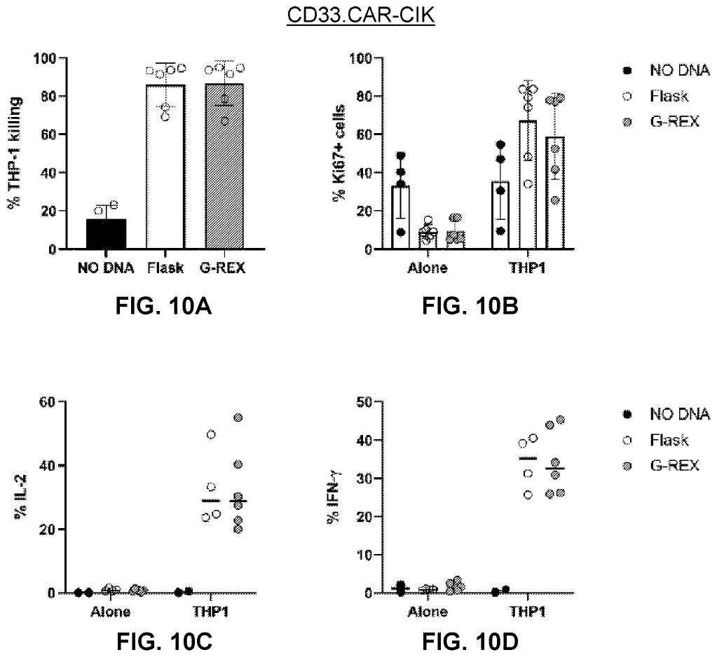

FIG. 10A shows that CAR-CIK cells generated by the Day 2 feeder free (either produced in Flasks or G-REX) display high lytic activity against a CD33+ cell line.

FIG. 10B shows CD33.CAR-CIK cells generated by the Day2 feeder free (either produced in Flasks or G-REX) proliferate in a CAR-specific manner when challenged with a CD33+ cell line

FIGS. 10C-10D show that CD33.CAR-CIK cells generated by the Day 2 feeder free process (either produced in Flasks or G-REX) produce IL-2 (FIG. 10C) and IFN-γ (FIG. 10D) in a CAR-specific manner when challenged with a CD33+ cell line.

In vivo Experiment

CD19.CAR-CIK cells produced with the Day 2 feeder free process and cultured in G-REX-day7 were tested in vivo, by exploiting a model of CD19+ DAUDI-engrafted in NOD scid gamma (NSG) mice (Jackson Laboratory). A dose finding titration was done, considering the doses of 5×10\hat{ }6 (G-REX 5) and 10×10\hat{ }6 (G-REX 10) CAR+ cells/mouse. Briefly, NSG mice were irradiated at sub-lethal doses of 200 rad and 0.5×10\hat{ }5 DAUDI cells (ATCC (American Type Culture Collection); Manassas, VA)/mouse were infused. Two days after, CD19.CAR-CIK cells were infused at different cell doses, and mice were followed during the days.

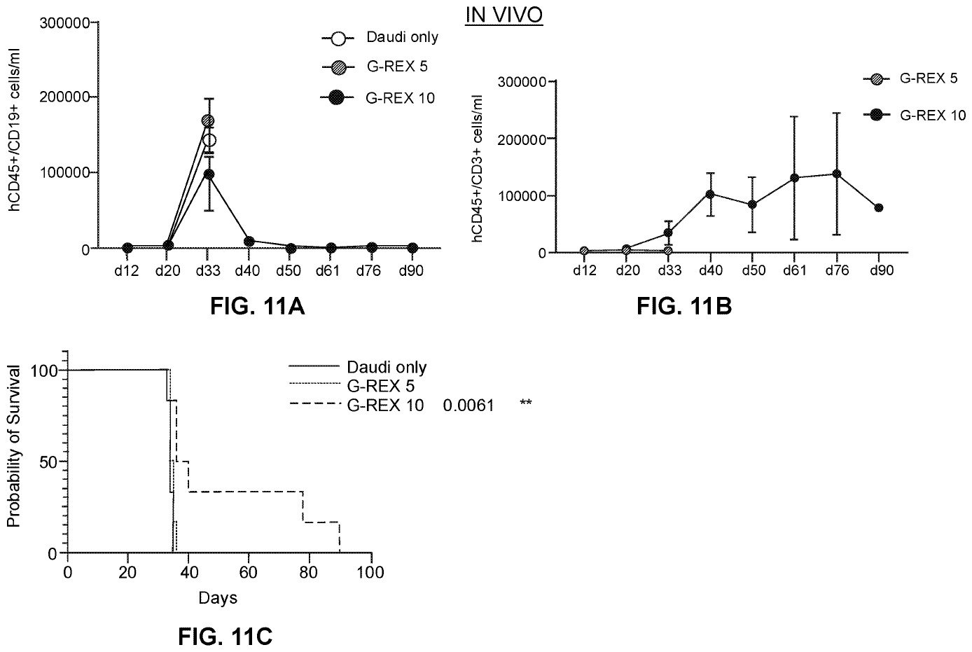

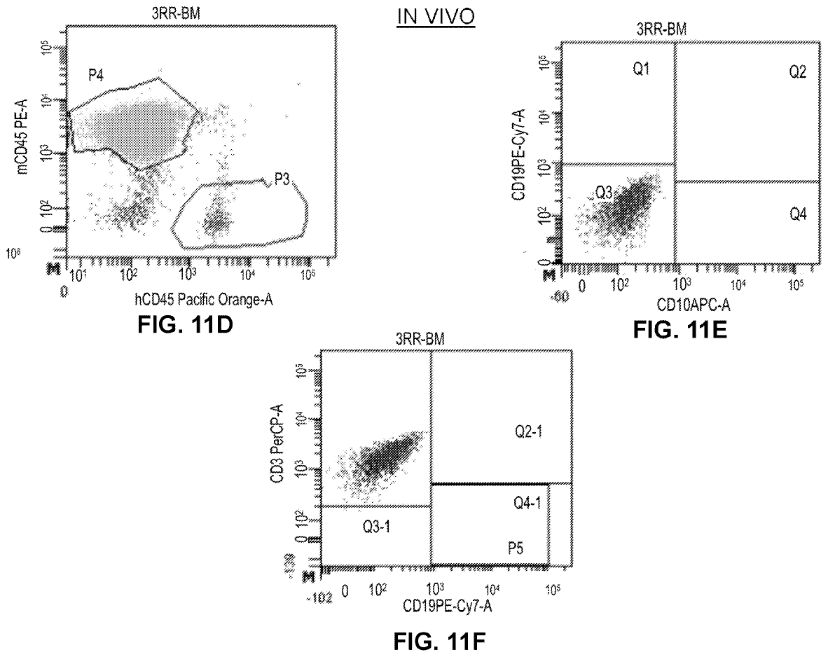

FIG. 11C shows that the dose of 10×10\hat{ }6 CAR+ cells/mouse showed a better tumor control as compared to the lowest dose of 5×10\hat{ }6 cells/mouse. FIG. 11E shows that the mouse was treated with the 10×10\hat{ }6 CD19.CAR-CIK cell dose, and no DAUDI cells were detected at the endpoint sacrifice analysis. FIG. 11F shows presence of CD3+ cells at the endpoint sacrifice analysis. In conclusion, mice treated with the highest dose (10×10\hat{ }6 CAR+ cells/mouse) showed a lower tumor burden, in parallel with a better expansion of CAR-CIK cells and a prolonged survival.

### Example 3

### Purity of CAR-CIK Cells

CIK cell is a population of CD3 cells bearing CD56 and NKG2D markers. Other cell types remaining at the end of CAR-CIK-CD19 cell production are composed of B cells, NK cells and myeloid cells. To characterize the cellular make up of cultures during the manufacturing cycle and on the day of harvest, samples were taken on day 0 and throughout the culture period described in Example 1 above. Samples were analyzed using antibodies specific for different cell types; CD3 (T cell marker), CD56/CD16 (NK cell marker), CD19 (B cell marker) CD14 and CD15 (myeloid cell markers).

Fluorochrome conjugated antibodies were used to stain cells for the following markers; for T cells anti-CD3 (antibody clone UCHT1), for NK cells anti-CD56 (antibody clone NCAM1) and anti-CD16 (antibody clone B73.1), for B cells anti-CD19 (antibody clone HIB19), for myeloid cells anti-CD14 (antibody clone MφP9) and anti-CD15 (antibody clone HI98) either on PBMC (day 0) or on CARCIK-CD19 cells during the culture period (Days 7, 14 and 21). Day 0 PBMC were isolated from whole blood collections by ficoll gradient centrifugation and the percentages of CD3+ T cell, CD56+/CD16+NK cells, CD19+ B cells, CD14+ and CD15+ myeloid cells were determined on the viable CD45 positive cell population. CD45 positive cells were detected with anti-CD45 antibody clone HI30 which binds to all human mononuclear cells. Viable cells were identified by the addition of a fixable Aqua dye that labels only permeable dead cells leaving viable cells un-labeled. During CAR-CIK-CD19 cell production the percentages of CD3+ T cell, CD56+/CD16+NK cells, CD19+ B cells, CD14+ and CD15+ myeloid cells present at day 7, day 14 and day 21 was determined on the viable CD45 positive cell population. Cell fluorescence was measured on a flow cytometer, and data was analyzed using FlowJo version 10.8.1 software (TreeStar).

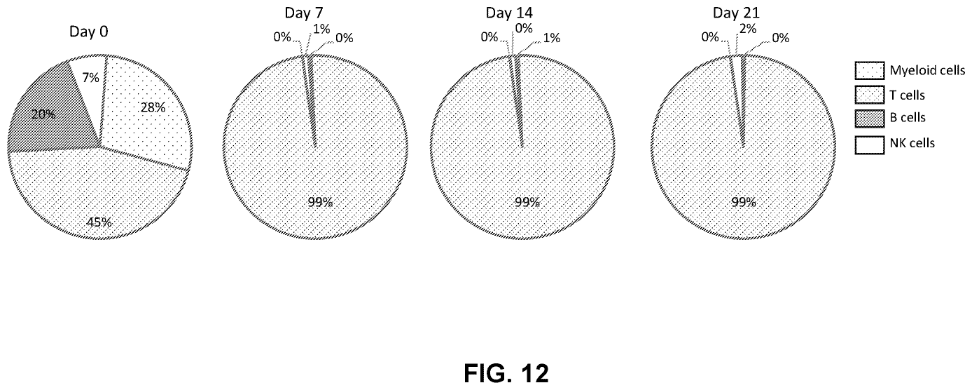

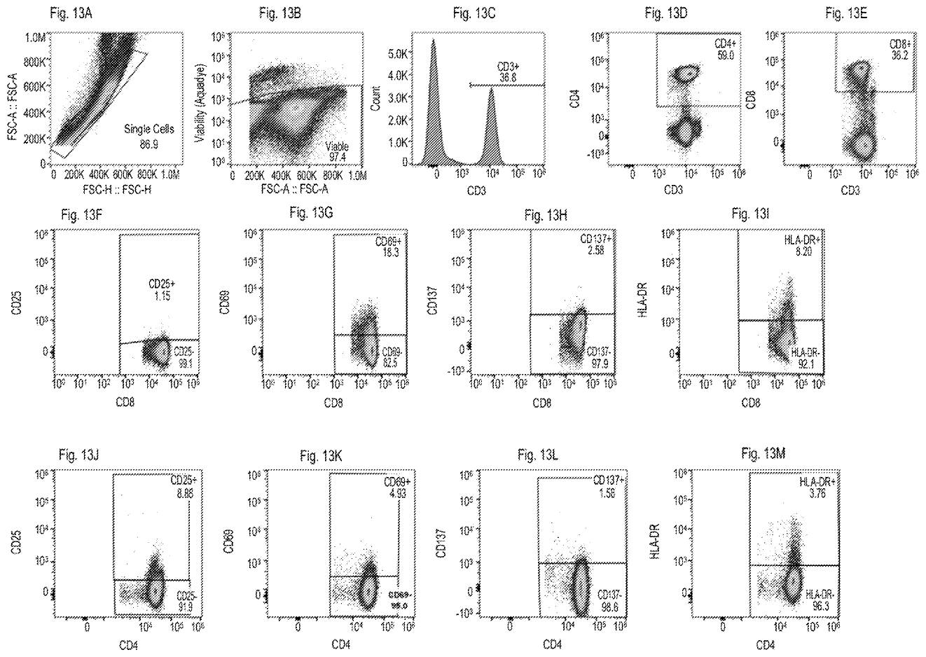

FIG. 12 shows data from a representative development run Development Run ID and the percentage of each population. Cells were harvested on days 0, 7, 14, and 21. This observation was repeated in a number of large scale pre-clinical runs with data summarized in Table 9.

TABLE 9
Summary of quantitation of residual cellular impurities
in four large scale development runs*

| | | Impurities, % | | |
|---|---|---|---|---|
| Development | | | | Monocytes and |
| Run ID | CD3+ cells | NK cells | B lymphocyte | Granulocyte |
| 1 | 97.9 | 1.6 | 0.01 | 0.4 |
| 2 | 98.7 | 0.5 | 0.00 | 0.1 |
| 3 | 97.9 | 1.6 | 0.00 | 0.2 |
| 4 | 96.6 | 2.5 | 0.00 | 0.4 |

*CAR-CIK cells were engineered using the SB transposon system using 3^{rd} generation pT4 CD19CAR plasmid.

These data demonstrate that at the end of manufacturing cycle (day 21) the majority of the cellular population is comprised of CAR-CIK-CD19 cells that are CD3 positive with little to no residual cell subsets.

### Example 4

Characterization of CIK cells During the Two Days of CAR-CIK Manufacturing Process

Cytokine induced killer (CIK) cells are activated cells derived from peripheral blood mononuclear cells (PBMCs) by the sequential addition of cytokines, such as IFN-γ and then stimulation through the CD3 receptor with the addition of IL-2. Based on the fact that day 2 and day 7 cells still have high CD28 and CCR7 together with the fact that the cells did not yet upregulate CD56 and NKG2D markers, they cannot be called CIK cells yet. However, these activated day 2 cells no longer require any modification with additional stimuli and are committed to CIK phenotype during their proliferation. As used herein, the term for these cells is “Committed CIK Precursor cells.” Furthermore, by day 2, activation of Committed CIK Precursor cells renders them permissible for delivery of CAR transgene gene products via electroporation. However, the activation state of the cell prior to electroporation can influence efficiency of transgene delivery. Therefore, a study was undertaken to characterize the activation state of day 2 activated cells prior to electroporation by tracking the expression of the following surface markers: CD25, CD69 and CD137, which are upregulated on the cell surface. The CD25 marker is the receptor for IL-2 and responsible for cell proliferation, CD69 allows activated cells to interact with antigen presenting cells, transducing growth signals, and CD137 is a costimulatory receptor responsible for memory cell differentiation. HLA-DR is an MHC class II molecule involved in antigen presentation and upregulated during cell activation. Tracking the cell surface expression of these markers characterized the activation state of the cell prior to electroporation.

### Methods

To generate CIK cells, PBMC were isolated from whole blood collection by ficoll gradient centrifugation and seeded at 3×10\hat{ }6 cells/ml in advanced RPMI/10% FBS. Activation was accomplished by the sequential addition of the cytokine IFN-g (1000 U/mL) on day 0 followed by stimulation through the CD3 receptor via an anti-CD3 specific antibody at 50 ng/ml (clone OKT3) plus the cytokine IL-2 (300 U/mL) on day 1 as described in Example 1 above. For generation of gene modified CIK cells, the activated cells were harvested on day 2 for electroporation with DNA plasmid pT4-CD19-CAR encoding the CAR transgene and SB100X transposase RNA (Capped SB100X). To characterize the activation state of cells prior to electroporation, multi-color flow cytometry was used to identify distinguishing cell surface marker proteins expressed by the activated cells.

### Methods and Gating Strategy to Identify CIK Cell Surface Marker Expression

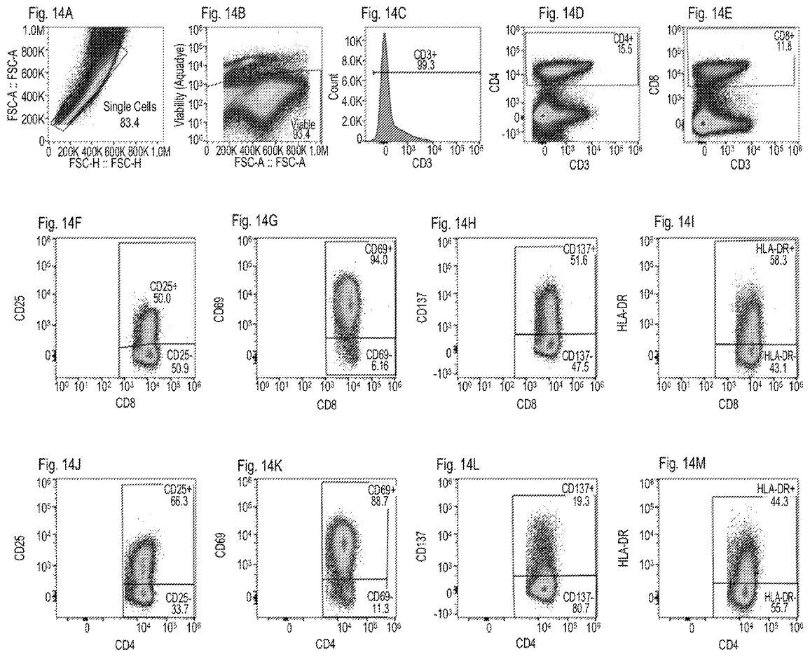

Prior to stimulation bulk PBMCs (day 0 cells) or activated day 2 cells were stained with the indicated antibodies listed in Table 10. Viable cells were identified by the addition of a fixable Aquadye that labels only permeable dead cells leaving viable cells un-labeled. Viable cells labeled with the indicated antibodies were then acquired on a flow cytometer and data was analyzed using FlowJo version 10.8.1 software (TreeStar). The gating strategy is shown in FIGS. 13A-13M for day 0 cells and FIGS. 14A-14M for day 2 cells. Single cells were identified by gating on forward scatter height (FSC-H) and forward scatter height area (FSC-A) (FIG. 13A and FIG. 14A). Viable cells were gated on the Aquadye-negative cell population (FIG. 13B and FIG. 14B). The viable cell population was gated on CD3+ cell events for day 0 PBMCs (FIG. 13C). The CD4+ cell population (FIG. 13D) and CD8+ cell population (FIG. 13E) were gated within CD3+ cell population. The activation markers CD25, CD69, CD137, and HLA-DR area were reported for both CD8+ cells (FIG. 13F, FIG. 13G, FIG. 13H, FIG. 13I, respectively) and CD4+ cells (FIG. 13J, FIG. 13K, FIG. 13L, FIG. 13M, respectively).

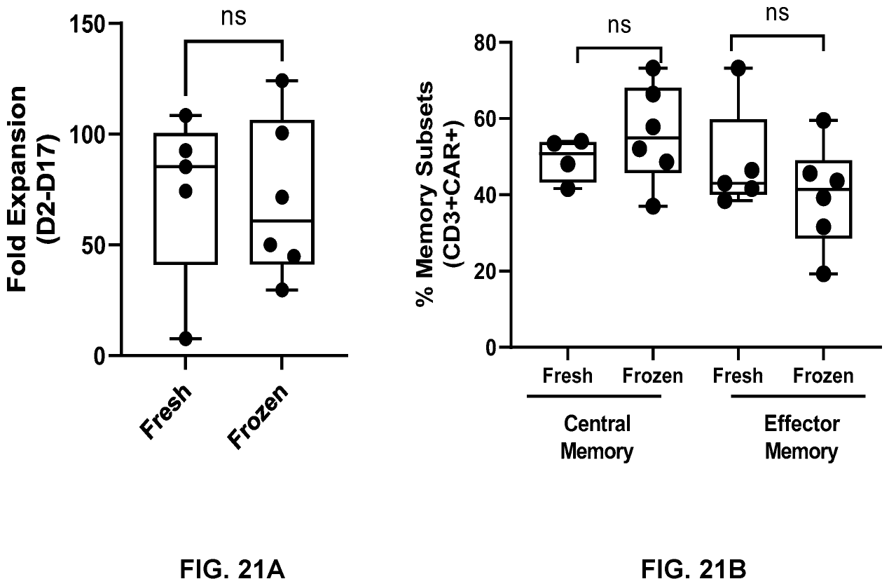

On day 2 of culture, detection of CD3 was prevented by the anti-CD3 (OKT3) antibody used to stimulate the CIK cells blocking the anti-CD3 antibody used to detect CD3 surface expression. Therefore, all events in the viable cell gate were included for the day 2 sample analysis (FIG. 14C). The CD4+ cell population (FIG. 14D) and CD8+ cell population (FIG. 14E) The activation markers CD25, CD69, CD137, and HLA-DR area were reported for both CD8+ cells (FIG. 14F, FIG. 14G, FIG. 14H, FIG. 14I, respectively) and CD4+ cells (FIG. 14J, FIG. 14K, FIG. 14L, FIG. 21M, respectively).

TABLE 10
Antibody clones for detection of CIK cell surface markers

| Surface | | Fluorochrome | Antibody |
|---|---|---|---|
| marker | Marker description | conjugate | Clone |
| CD3 | Cell signaling receptor | CD3 APC-efluor 780 | UCHT1 |
| CD4 | Helper cell marker | CD4 FITC | SK3 |
| CD8 | Cytotoxic cell marker | CD8 PE | HIT8a |
| CD25 | Receptor for IL-2 | CD25 Alexa Fluor 700 | MEM-181 |
| CD69 | C-type lectin, early | CD69 Pacific Blue | FN50 |
| | activation marker | | |
| CD137 | Costimulatory receptor | CD137 PE Cy7 | 4B4-1 |
| HLA-DR | MCH class II cell | HLA-DR APC | L243 |
| | surface receptor | | |

### Results

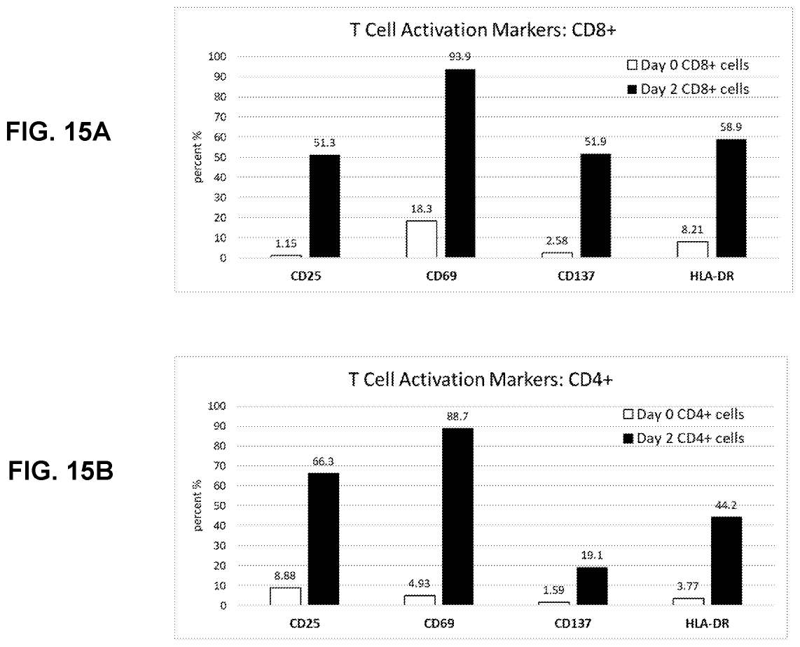

The PBMC population consists of monocytes, B cells, CD3+/CD4+ T cells, and CD3+/CD8+ T cells. All cells that will become CIK cells at the end of manufacturing process can be characterized by the CD3 marker. Those cells can be further divided to CD4 cells and CD8 Cells. The process of commitment to CIK cell phenotype is achieved by the sequential addition of IFN-γ followed by anti-CD3 stimulation plus IL-2. FIGS. 15A and 15B show data from one representative lot. Low levels of the activation markers CD25, CD69, CD137 and HLA-DR were detected on both CD8+ cells (FIG. 15A) and CD4+ cells (FIG. 15B). However, on day 2 post activation all the markers were upregulated on both CD8+ cells and CD4+ cells (FIGS. 15A and 15B). Table 11 shows data collected at day 0 on both CD8+ cells and CD4+ cells from eight separate cellular production runs. Table 12 shows data collected at day 2 post activation on both CD8+ cells and CD4+ cells from eight separate cellular production runs, with lot number PD008-15 being split into 2 additional arms A and B for a total of nine samples. HLA-DR was only tested on five out of the nine samples on day 2.

A similar trend was seen for both CD8+ and CD4+ cells, with upregulation of all markers tested (Table 12). CD25 expression was upregulated to 41.8% to 88.5% for both CD8+ cells and CD4+ cells. CD69 was upregulated to greater than 80%, with some lots of cells reaching greater than 90% for both CD8+ cells and CD4+ cells. Overall, a higher percentage of CD8+ cells expressing CD137 (range of 46.4% to 88.8%) was seen compared to CD4+ cells (range of 10.4% to 60.9%). HLA-DR was upregulated on both CD8+ cells and CD4+ cells to ranges of 50% to 80.2% and 30.1% to 78.9% respectively.

Table 13 shows the min, max and median values derived at day 0 and day 2 for all the markers tested. To set an acceptable threshold of upregulation for each marker from day 0 to day 2 post activation, the change in the median values from day 0 to day 2 was reported as a percent increase in surface marker expression. Therefore, values greater than 50% expression for CD25, CD69, CD137, and HLA-DR on CD8+ cells indicated upregulation of these markers post activation. Values greater than 60% expression for CD25 and CD69 on CD4+ cells indicated upregulation of CD25 and CD69 post activation. With regards to CD137 and HLA-DR, on activated CD4+ cells, given the lower median values of 34.8% and 44.3% respectively, values greater than 30% would be considered an acceptable upregulation of CD137 and HLA-DR on CD4+ cells. Given the broad range of upregulation of the four activation markers post stimulation, day 2 Committed CIK Precursor cells exhibit an activated phenotype that would render them applicable for electroporation.

TABLE 11
Detection of activation markers on day 0 cells*

| | | Lot number | | | | | | | |
|---|---|---|---|---|---|---|---|---|---|
| | Marker | PD008-09 | PD008-10 | PD008-11 | PD008-12 | PD008-13 | PD008-14 | PD-008-15 | PD-008-16 |
| CD8+ | CD25 | 3.5 | 1.2 | 1.3 | 1.2 | 0.8 | 0.4 | 0.7 | 0.2 |
| cells | CD69 | 12.7 | 13.6 | 17.2 | 18.3 | 10.5 | 5.6 | 8.6 | 3.3 |
| of the | CD137 | 16.6 | 2.9 | 4.9 | 2.6 | 1.6 | 3.0 | 14.2 | 1.3 |
| viable | HLA-DR | 11.1 | 3.2 | 3.4 | 8.2 | 12.8 | 0.8 | 2.5 | 0.2 |
| CD3+ | | | | | | | | | |
| cells | | | | | | | | | |
| CD4+ | CD25 | 12.4 | 6.2 | 4.6 | 8.9 | 1.6 | 1.4 | 2.7 | 1.5 |
| cells | CD69 | 15.5 | 5.3 | 6.7 | 4.9 | 10.6 | 3.2 | 4.8 | 1.9 |
| of the | CD137 | 16.7 | 1.0 | 1.7 | 1.6 | 1.8 | 0.6 | 7.4 | 0.7 |
| viable | HLA-DR | 11.7 | 2.3 | 2.2 | 3.8 | 47.6 | 2.6 | 34.2 | 0.8 |
| CD3+ | | | | | | | | | |
| cells | | | | | | | | | |

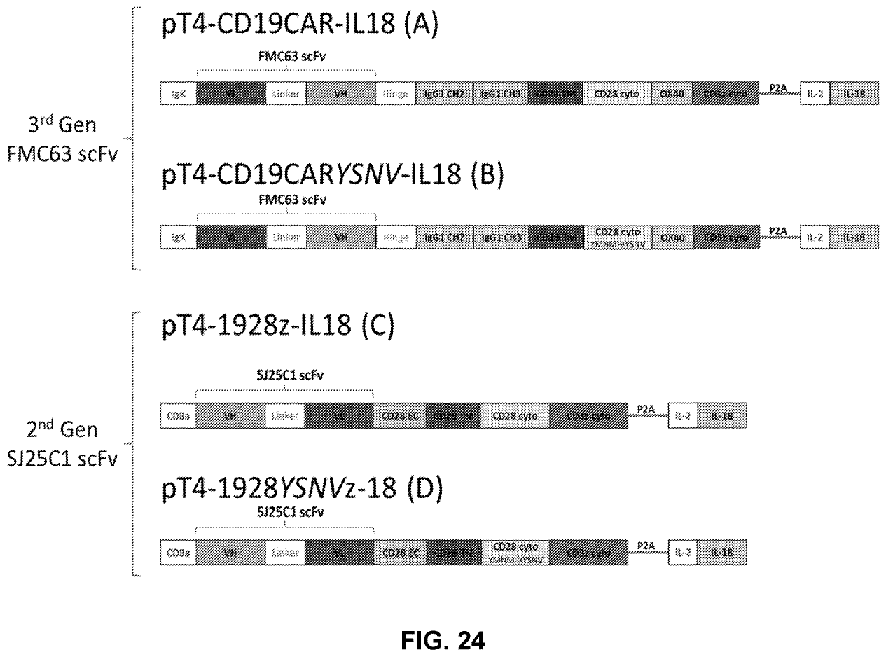

The percentage of the activation markers CD25, CD69, CD137 and HLA-DR within the CD8+ and CD4+ viable CD3+ cell population for each lot of PD008 cellular material is shown for day 0.
*CAR-CIK cells were engineered using the SB transposon system using 3^{rd} generation pT4 CD19CAR-IL18 bicistronic plasmid (see (FIG. 24, pT4-CD19CAR-IL18 plasmid A).

TABLE 12
Detection of activation markers on day 2 cells*

| | | Lot number | | | | | | | | |
|---|---|---|---|---|---|---|---|---|---|---|
| | Marker | PD008-09 | PD008-10 | PD008-11 | PD008-12 | PD008-13 | PD008-14 | PD008-15a | PD008-15b | PD008-16 |
| CD8+ | CD25 | 52.4 | 41.8 | 44.3 | 50.0 | 73.5 | 82.5 | 56.0 | 66.4 | 7.8 |
| cells | CD69 | 94.7 | 94.4 | 98.3 | 94.0 | 92.7 | 98.6 | 98.3 | 98.7 | 89.2 |
| of the | CD137 | 73.5 | 46.4 | 53.6 | 51.6 | 78.3 | 88.8 | 79.7 | 77.1 | 58.3 |
| viable | HLA-DR | 61.5 | 50.0 | 53.0 | 58.3 | 80.2 | nt | nt | nt | nt |
| CD3+ | | | | | | | | | | |
| cells | | | | | | | | | | |
| CD4+ | CD25 | 57.0 | 40.3 | 64.3 | 66.3 | 82.0 | 88.5 | 75.8 | 79.7 | 38.2 |
| cells | CD69 | 89.6 | 81.6 | 96.0 | 88.7 | 95.0 | 96.4 | 96.1 | 97.2 | 89.1 |
| of the | CD137 | 29.3 | 10.4 | 11.8 | 19.3 | 37.4 | 60.9 | 48.1 | 46.3 | 34.8 |
| viable | HLA-DR | 51.3 | 30.1 | 30.4 | 44.3 | 78.9 | nt | nt | nt | nt |
| CD3+ | | | | | | | | | | |
| cells | | | | | | | | | | |

The percentage of the activation markers CD25, CD69, CD137 and HLA-DR within the CD8+ and CD4+ viable CD3+ cell population for each lot of PD008 cellular material is shown for day 2. nt = not tested.
*CAR-CIK cells were engineered using the SB transposon system using 3^{rd} generation pT4 CD19CAR-IL18 bicistronic plasmid (see (FIG. 24, pT4-CD19CAR-IL18 plasmid A).

TABLE 13
Min, max and median values for surface marker expression*

| | | | | | | | | Increase in |
|---|---|---|---|---|---|---|---|---|
| | | | | | | | | median |
| | CD8+ cells Day 0 | | | | CD8+ cells Day 2 | | | value Day |
| | Min | Max | Median | | Min | Max | Median | 0 to Day 2 |
| Marker | (%) | (%) | (%) | Marker | (%) | (%) | (%) | (% of cells) |
| CD25 | 0.2 | 3.5 | 1 | CD25 | 7.8 | 82.5 | 52.4 | 51.4 |
| CD69 | 3.3 | 18.3 | 11.6 | CD69 | 89.2 | 98.7 | 94.7 | 83.1 |
| CD137 | 1.3 | 16.6 | 3.0 | CD137 | 46.4 | 88.8 | 73.5 | 70.5 |
| HLA-DR | 0.2 | 12.8 | 3.3 | HLA-DR | 50 | 80.2 | 58.3 | 55.0 |
| | CD4+ cells Day 0 | | | | CD4+ cells Day 2 | | | |
| CD25 | 1.4 | 12.4 | 3.7 | CD25 | 38.2 | 88.5 | 66.3 | 62.6 |
| CD69 | 1.9 | 15.5 | 5.1 | CD69 | 81.6 | 97.2 | 95 | 89.9 |
| CD137 | 0.6 | 16.7 | 1.7 | CD137 | 10.4 | 60.9 | 34.8 | 33.1 |
| HLA-DR | 0.8 | 47.6 | 3.2 | HLA-DR | 30.1 | 78.9 | 44.3 | 41.1 |

The minimum and maximum values are reported for cell lot number values shown in table 11 for day 0 and table 12 for day 2. The median was calculated for the tested lot numbers. The percent increase from day 0 to day 2 was calculated by subtracting the value reported on day 0 from the value reported for day 2 for each marker and represents the percent increase in surface marker expression post activation.
*CAR-CIK cells were engineered using the SB transposon system using 3^{rd} generation pT4 CD19CAR-IL18 bicistronic plasmid (see (FIG. 24, pT4-CD19CAR-IL18 plasmid A).

### Example 5

### Use of Feeder Free Cells for Electroporation Delivery of CAR Transgene Products

As discussed above in Example 4, Committed CIK Precursor cells are activated cells derived from peripheral blood mononuclear cells (PBMCs) by the sequential addition of cytokines, such as IFN-γ and then stimulation through the CD3 receptor with the addition of IL-2. By day 2, activation of these cells renders them permissible for delivery of CAR transgene gene products and further expansion in culture without the addition of feeder cells. The delivery of the CAR transgene to feeder free activated cells using the sleeping beauty transposase (SB100X) encoded either by plasmid DNA or as RNA was tested. Co-delivery of the CAR transgene and SB100X transposase by electroporation after 2 days of activation resulted in stable surface expression of the CAR transgene on CD3+ cells through a 21 day culture period. The successful delivery of the CAR transgene to feeder free activated cells using the sleeping beauty transposase (SB100X) encoded either by plasmid DNA or as RNA was shown. Furthermore, it was shown that both SB100X RNA and SB100×DNA plasmid encoded transposase, different electroporation devices and buffers can be used to facilitate successful CAR transgene delivery under the conditions tested.

### Methods

PBMCs were isolated by ficoll gradient centrifugation from leukapheresis products. Isolated PBMCs were frozen in FBS with 10% DMSO and stored under liquid nitrogen. PBMCs were thawed, washed with advanced RPMI containing 10% FBS media, resuspended at 3×10\hat{ }6 cell/mL in advanced RPMI/10% FBS media and stimulated by the sequential addition of the cytokine IFN-γ (1000 U/mL) on day 0 followed by stimulation through the CD3 receptor via an anti-CD3 specific antibody at 50 ng/ml (clone OKT3) plus the cytokine IL-2 (300 U/mL) on day 1. For generation of gene modified CIK cells, the activated cells were harvested on day 2 for electroporation with DNA plasmid pT4-CD19-CAR encoding the CAR transgene and SB100X transposase. The SB100X transposase is encoded by either plasmid DNA (pCMV-SB100X) or RNA (Capped SB100X). Day 2 activated cells were resuspended in different buffers for testing during electroporation with a BioRad Gene Pulser Xcell. Cells were resuspended at 1×10\hat{ }8cell/mL in either BioRad, CoStorSol, Lonza or OptiMEM buffers and 1×10\hat{ }7cells in 100 ml buffer were transferred to 2 mm gap cuvettes for electroporation. The transposase, plasmid, and cell concentration along with the electroporation voltages and buffers are listed in Table 14. The mock cells were electroporated in CoStorSol buffer in the absence of nucleic acids. Post electroporation, each cuvette of cells was added to a single well of a 6 well plate in 5 mL of complete media (Advanced RPMI/20% FBS). On day 3 media was exchanged by removing half of the media and replacing the volume with fresh media (advanced RPMI/10% FBS containing 600 U/mL IL-2, for a final concentration of 300 U/mL). On day 7, cells were counted and transferred to flasks and seeded at a density of 1×10\hat{ }6 cells/mL. On days 10, 14 and 17 cells were counted and reseeded at 1×10\hat{ }6/mL in flasks and then harvested on day 21. The advanced RPMI/10% FBS supplemented with 300 U/mL IL-2 was used for day 7 feedings onward.

TABLE 14
Electroporation Parameters*

| | | Electro- | pT4-CD19-CAR | SB100X DNA |
|---|---|---|---|---|
| | | poration | plasmid D-NA | pCMV-SB100X |
| Sample# | Voltage | buffer | mg/1 × 10\hat{ }7 cells | mg/1 × 10\hat{ }7 cells |
| | 250 | Mock | 0 | 0 |
| 1 | 250 | CoStorSol | 15 | 0.5 |
| 2 | 250 | OptiMEM | 15 | 0.5 |
| 3 | 250 | Lonza | 15 | 0.5 |
| 4 | 250 | BioRad | 15 | 0.5 |
| | | Electro- | pT4-CD19-CAR | Capped SB100X |
| | | poration | plasmid DNA | RNA |
| Sample# | Voltage | buffer | mg/1 × 10\hat{ }7 cells | mg/1 × 10\hat{ }7 cells |
| 5 | 350 | CoStorSol | 15 | 0.5 |
| 6 | 350 | OptiMEM | 15 | 0.5 |
| 7 | 350 | Lonza | 15 | 0.5 |
| 8 | 350 | BioRad | 15 | 0.5 |

Day 2 activated cells are harvested and washed and resuspended in the indicated electroporation buffers listed above. For electroporation with CAR plasmid DNA plus SB100X plasmid DNA the voltage was set at 250 V square wave 1 pulse 10 ms (samples 1-4). SB100X plasmid DNA was added to cells at a final concentration of 0.5 mg/1 × 10\hat{ }7 cells in 100 ml. For electroporation with CAR plasmid DNA plus SB100X RNA the voltage was set at 350 V square wave 1 pulse 10 ms (samples 5-8). SB100X RNA was added to cells at a final concentration of 0.5 mg/1 × 10\hat{ }7 cells in 100 ml. Mock cells were electroporated without nucleic acids and serve as controls to set gates for measuring CAR positive cells.
*CAR-CIK cells were engineered using the SB transposon system using 3rd generation pT4 CD19CAR plasmid.

### Results

Electroporation of day 2 activated cells in the absence of added feeder cells, with the combination of pT4-CD19-CAR plasmid DNA and pCMV-SB100X plasmid DNA resulted in the expression of the CAR transgene.

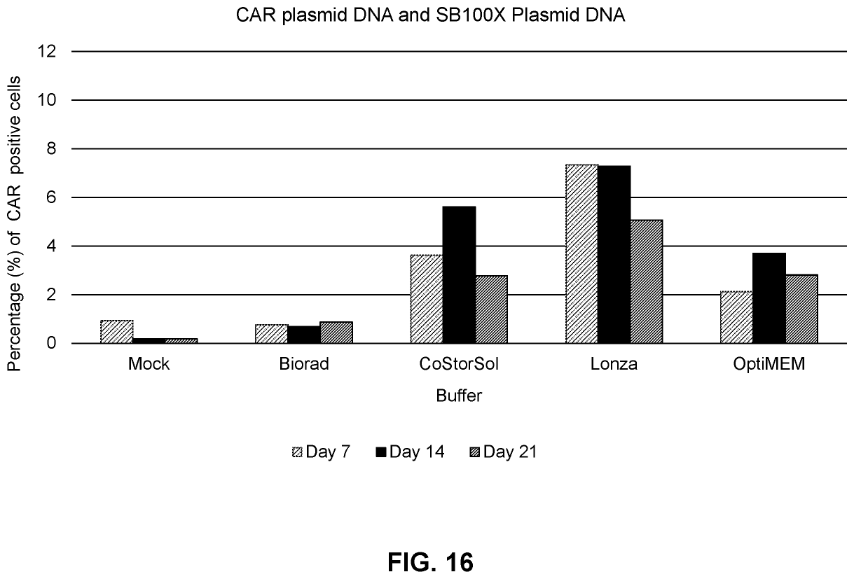

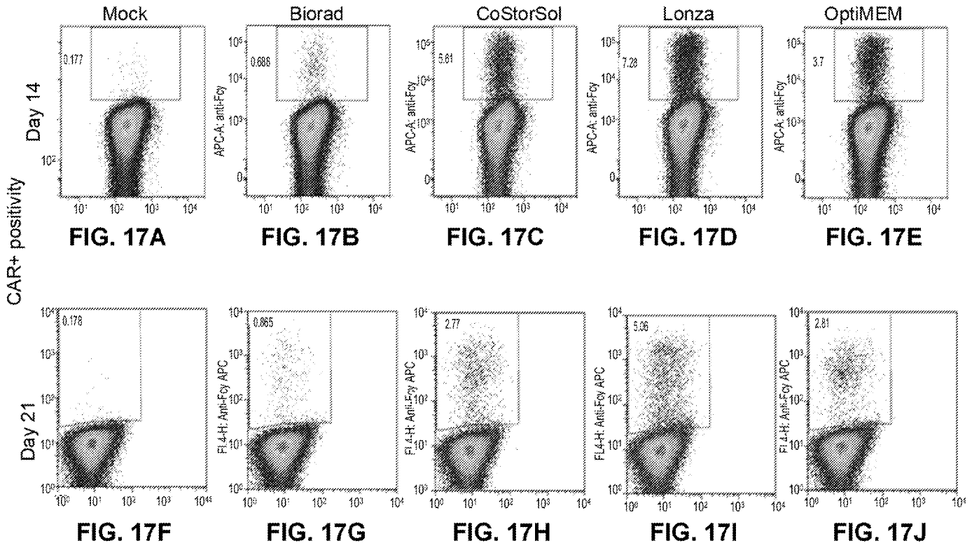

However, electroporation efficiency in this experiment was influenced by the buffer the cells are resuspended in for electroporation. Cells electroporated in CoStorSol, Lonza or OptiMEM buffer showed the presence of CAR positive cells by day 7 and this population remained present in the culture through days 14 and 21. Few CAR positive cells were seen in cells electroporated in the BioRad buffer at days 7, 14 or 21 (FIG. 16). FIGS. 17A-17J show the gating of CAR positive cells in the CD3+ cell gate at day 14 and 21. Activated cells electroporated in the Lonza buffer resulted in the highest expression of CAR positivity (7.28%) at day 14 (FIG. 17D). By day 21 the expression of CAR positivity diminishes but was still detectable above mock cells (FIG. 17F) for cells electroporated in CoStorSol (FIG. 17H), Lonza (FIG. 17I), or OptiMEM (FIG. 17J) buffers. Cells electroporated in the Lonza buffer resulted in the highest CAR positivity (5.06%) at day 21 (FIG. 17I). A small but detectable population of CAR positive cells was seen in cells electroporated in the BioRad buffer (0.688%) by day 14 (FIG. 17B) and day 21 (0.865%) (FIG. 17G). Data shown reveals that electroporating day 2 activated cells with a combination of pT4-CD19-CAR plasmid DNA and pCMV-SB100X plasmid DNA in CoStorSol, Lonza, or OptiMEM buffers resulted in expression of the CAR transgene by CD3+ cells.

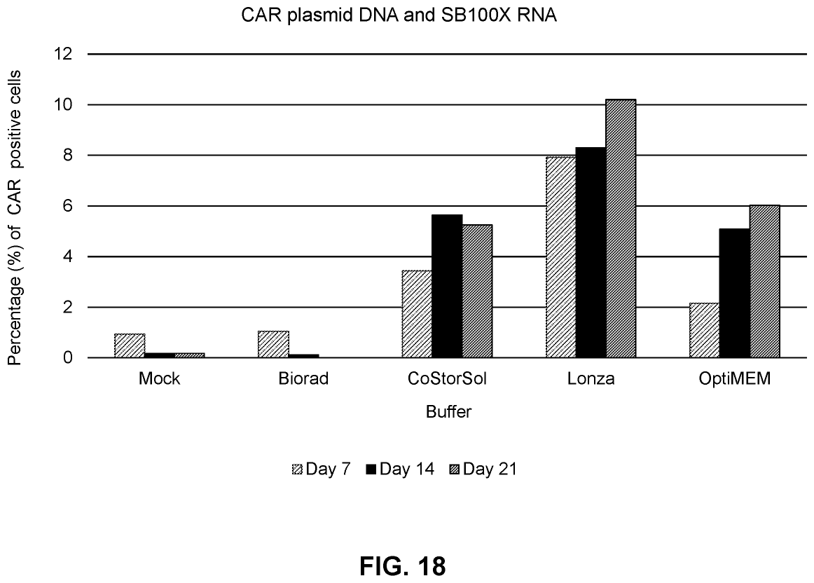

Day 2 activated cells were also electroporated with the combination of pT4-CD19-CAR plasmid DNA and SB100X RNA. Here the transposase was encoded by RNA instead of DNA. Electroporation of day 2 activated cells with the combination of pT4-CD19-CAR plasmid DNA and SB100X RNA resulted in the expression of the CAR transgene by day 7, with three out of four buffers, CoStorsol, Lonza and OptiMEM resulting in the sustained presence of CAR positive cells at days 14 and 21 of the culture period (FIG. 18). Similar to what was seen using the transposase encoded as plasmid DNA, there were few CAR positive cells when using the BioRad buffer over cells electroporated in the absence of nucleic acid (day 7; FIG. 18). Moreover, there was no expansion of CAR positive cells derived from using the BioRad buffer at days 14 and 21 day of the culture period (FIG. 18).

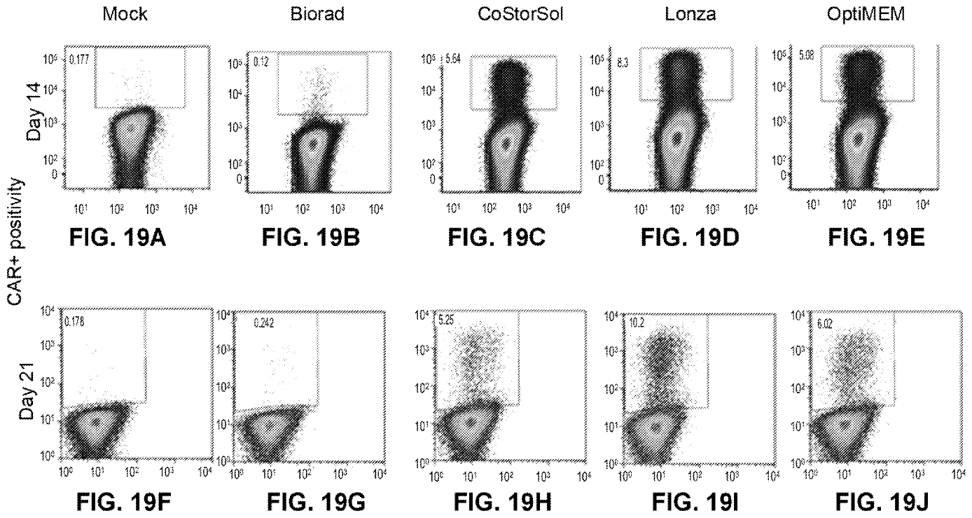

FIGS. 19A-19J show the gating of CAR positive cells in the CD3+ cell gate at days 14 and 21. Activated cells electroporated in the Lonza buffer resulted in the highest expression of CAR positivity (8.3%) on day 14 (FIG. 19D) and activated cells electroporated in CoStorSol or OptiMEM buffer having similar CAR positivity of 5.64% (FIG. 19C) and 5.08% (FIG. 19E), respectively. A similar pattern of CAR positivity was seen by day 21. Activated cells electroporated in the Lonza buffer maintained the highest CAR positivity of 10.2% (FIG. 19I) and activated cells electroporated in CoStorSol or OptiMEM had CAR positivity of 5.25% (FIG. 19H) and 6.02% (FIG. 19J), respectively. As reported with the SB100X transposase encoded by plasmid DNA, using the SB100X transposase as RNA resulted in similar pattern of CAR positive cells in the 21 day cultures. Electroporating day 2 activated cells with a combination of pT4-CD19-CAR plasmid DNA and Capped-SB100X RNA in CoStorSol, Lonza, or OptiMEM buffers resulted in expression of the CAR transgene by CD3+ cells.

Thus, the utility is shown of using feeder free derived activated cells as a cell source for electroporation with the transposase SB100X encoded as either DNA or RNA in combination with plasmid DNA encoding a CAR. In addition, the system of using feeder free activated cells for electroporation is amiable to different electroporation buffers, where activated cells expressing the CAR transgene post electroporation are propagated through a 21 day culture period.

### Example 6

Preparation of CAR-CIK Cells from Cord Blood

CAR-CIK cells were prepared from cord blood cells according to the following protocol. On day 0, cord blood cells obtained from frozen cord blood units were thawed. Cells were centrifuged and seeded in in 6-well plates at 3×10\hat{ }6/ml in 5 ml complete medium. 1000 U/ml of the cytokine IFN-γ was added. On day 1 (18 to 24 hours), IL-2 (300 U/ml) and the anti-CD3 antibody OKT3 (50 ng/ml) were added to the cultures. On day 2 (18 to 24 hours), cells were harvested, washed, and resuspend in electroporation buffer. Cells 10×10\hat{ }6/cuvette were mixed with DNA/RNA and electroporated. Post electroporation, one electroporated reaction was plated per well of 6-well plate in 4 ml of complete medium. On day 3 (18 to 24 hours), a half of the medium was replaced with complete medium+300 U/ml IL-2. On day 7, cells were passaged into flasks at 1×10\hat{ }6/ml in complete medium+300 U/ml IL-2. Cell cultures were fed every 3 to 4 days. On Days 10/11, 13/14 and 17/18, cells were split in flasks at 1×10\hat{ }6/ml in complete medium+300 U/ml IL-2. On day 21, the following analyses were performed: phenotype analysis, VCN and transposase expression was measured by validated quantitative PCR and RT PCR, and function (cytotoxicity assay, cytokines, proliferation) as described in Example 1 above and the cells were frozen.

### Example 7

Preparation of CAR-CIK Cells from Fresh Vs Frozen PBMCs

The use of cryopreserved PBMCs for the manufacturing of CARCIK-1918 cells was evaluated. Cryopreserving of PBMC allows for a more flexible cell manufacturing process, providing greater logistical flexibility to better accommodate scheduling for leukapheresis, shipping, and manufacturing (Wang et al. Oncolytics 3:16015 (2016)).

PBMCs from healthy donors were isolated from leukapheresis products and processed either fresh or after cryopreservation. Briefly, cells were cultured with IFN-γ (day 0), stimulated with OKT3 and IL-2 (day 1), electroporated with SB100X RNA and pT4-1928YSNVz-IL18 plasmid (see FIG. 24, plasmid D) (day 2), and expanded in culture for 15 days, as described in Example 1 above.

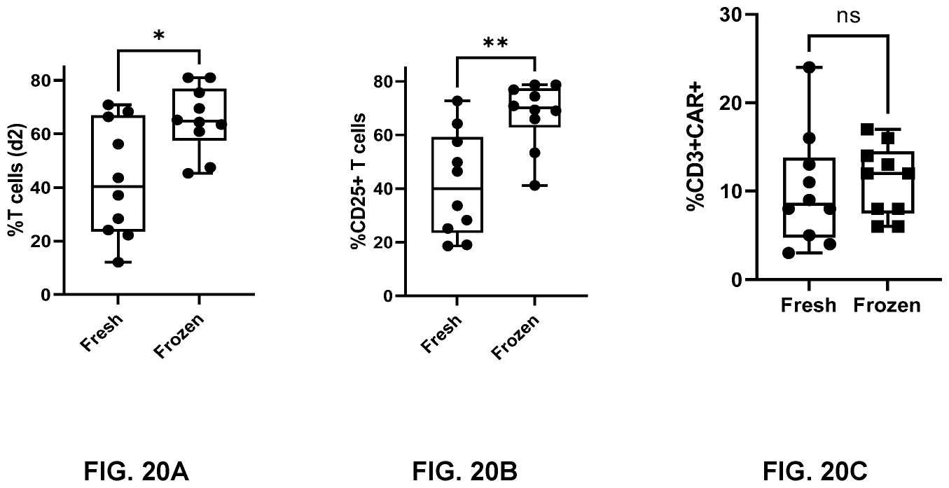

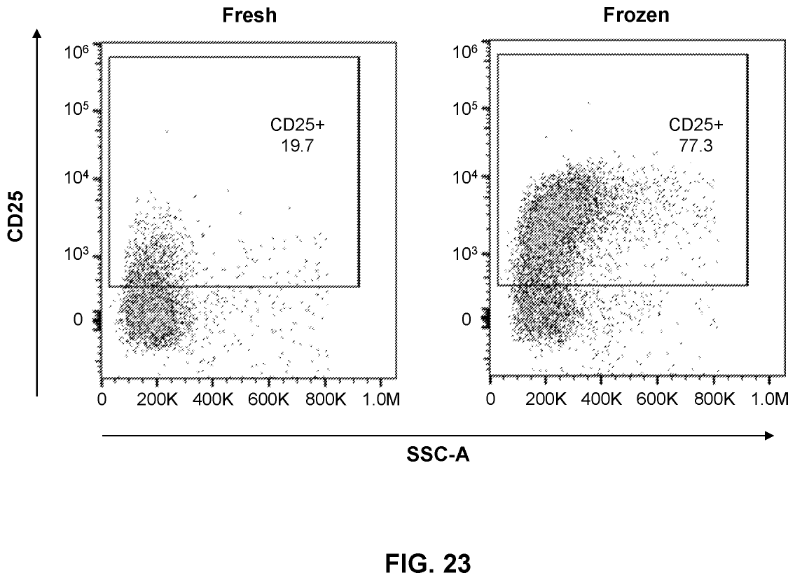

T cell enrichment and T cell activation were compared before electroporation in lots initiated from cryopreserved versus fresh PBMCs. On day 2, cells were analyzed using flow cytometry to determine the % of T cells and the expression of the activation marker CD25 in those T cells. PBMC cryopreservation resulted in a significantly higher T cell enrichment (% of T cells in the culture) (FIG. 20A) and higher T cells activation (% CD25+ T cells) (FIG. 20B and FIG. 23). On day 7, CAR expression was measured using flow cytometry to determine transfection efficiency. No significant differences in CAR expression were seen when using cryopreserved (frozen) or fresh PBMCs, but the CAR transduction efficiency was more consistent when using cryopreserved PBMCs (Standard deviation Fresh=% 6.33 vs Frozen=% 3.99) (FIG. 20C).

The effect of PBMC cryopreservation on the expansion of CARCIK-1918 cells was also evaluated. Cell expansion was calculated from day 2 (electroporation) to day 17 (end of processing). FIG. 21A shows that cryopreservation of PBMCs does not impact cell expansion during CARCIK-1918 manufacturing process.

The memory phenotype of the final product CARCIK-1918 cells was evaluated by assessing the expression of CD62L, CD45RO markers on CD3+CAR+ cells. Central memory cells are identified as CD62L+CD45RO+, while effector memory cells are identified as CD62L-CD45RO+. FIG. 21B shows that cryopreservation of PBMCs does not impact memory phenotype of CARCIK-1918 cells.

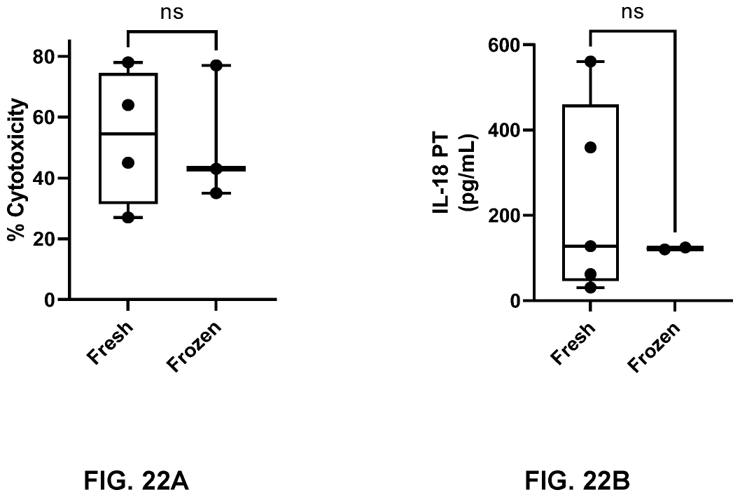

Finally, the effect of PBMC cryopreservation in the function of the final product CARCIK-1918 cells was assessed. The final product CARCIK-1918 cells manufactured from either fresh or cryopreserved (frozen) PBMCs were characterized by in vitro stimulation with CD19+ REH tumor cells to assess tumor killing activity and IL-18 secretion. CARCIK-1918 cells demonstrated potent in vitro cytotoxicity and IL-18 secretion towards the CD19+ REH target cell line. No significant differences were detected in products manufactured from fresh or frozen PBMCs (% cytotoxicity (FIG. 22A) and IL-18 secretion (FIG. 22B)).

The use of frozen PBMCs results in high electroporation rates and the cell phenotype, number, and potency by the end of the production process is comparable to the products initiated from fresh PBMCs.

### Example 8

### In Vivo Evaluation of CARCIK Cells in CD19+ Human Burkitt's Lymphoma Mouse Model (NSG/Raji)

The anti-tumor efficacy of CARCIK-1918 cells was evaluated in the CD19+ human Burkitt's lymphoma model generated by xeno-transplantation of the human Raji cell line into NOD scid gamma (NSG) immunocompromised mice.

CARCIK-CD19 cells with three modifications to the CD19.CAR DNA plasmid construct were generated to enhance in vivo anti-tumor activity and cell persistence.

The first modification incorporated the gene for IL-18 was linked to the CAR transgene in a bicistronic DNA plasmid used to generate CARCIK-CD19 cells.

The second modification for the CAR construct design was to test two versions of the CAR transgene. A 3^{rd} generation CAR which uses the CD19 antigen recognition domain derived from the FMC63 monoclonal antibody (mAb) (Jackson ImmunoResearch laboratories, catalog #109-136-098), joined to the CD28 transmembrane domain with an intracellular CD28-OX40-CD35 signaling domain. (See e.g., Magnani et al. J Clin Invest. 130 (11): 6021-6033 (2020)). The other CAR transgene evaluated is a 2^{nd} generation CAR which uses a CD19 antigen recognition domain derived from the SJ25C1 mAb (Memorial Sloan Kettering Cancer Center (MSKCC) Antibody Core Facility) joined to a CD28 transmembrane domain with an intracellular CD28-CD3z signaling domain. (See e.g., Brentjens et al. Blood 118 (18): 4817-4828 (2011); Park et al. N Engl J Med. 378 (5): 449-459 (2018)). Therefore, both 2^{nd} and 3^{rd} generation CAR.CD19 constructs were generated in a bicistronic DNA plasmid with the IL-18 gene.

The third modification incorporated an amino acid change to the CD28 cytoplasmic signaling domain. Specifically, the YMNM motif of the CD28 signaling domain was substituted to YSNV (See e.g., International Publ. No. WO2021158850A1) in order to attenuate CD28 signaling. Therefore, both 2^{nd} and 3^{rd} generation CAR.CD19 IL-18 bicistronic constructs were generated with the YSNV CD28 signaling motif.

Four DNA plasmids were designed incorporating either 2^{nd} or 3^{rd} generation CAR transgenes all with the IL-18 gene as a bicistronic construct (FIG. 24). The first two contained the 3^{rd} generation CAR transgene with either the parental CD28 signaling domain (FIG. 24, pT4-CD19CAR-IL18 plasmid A) or the signaling domain with the amino acid substitution CD28-YSNV (FIG. 24, pT4-CD19CARYSNV-IL18 plasmid B). The constructs with the 2^{nd} generation CAR transgene contained either the parental CD28 signaling domain (FIG. 24, pT4-1928z-IL18 plasmid C) or the signaling domain with the amino acid substitution CD28-YSNV (FIG. 24, pT4-1928YSNVz-18 plasmid D). Donor-derived CIK cells were engineered using the SB transposon system to express the CD19.CAR-IL18 transgenes and are referred to as “CARCIK-1918” cells. CARCIK-CD1918 cells were produced from 2 independent donors and tested in tumor bearing NOD scid gamma (NSG) mice (Roswell Park COSR facility, Buffalo. NY). Selection of the optimal CAR.CD19 transgene was based on the ability of CARCIK-CD1918 cells to control tumor growth, enhance animal survival, and cell persistence in vivo. Table 15 outlines the four sets of CARCIK-1918 cells tested each generated with the indicated CAR transgene plasmid DNA. The test lots of cells were referred to as Arm A, Arm B, Arm C, and Arm D.

TABLE 15
List of the Four Arms of CARCIK-1918
cells containing each plasmid

| | | | | CD28 |
|---|---|---|---|---|
| | CAR | | | signaling |
| Arm | generation | Plasmid | scFv | domain |
| Arm A | 3^{rd} gen | pT4-CD19CAR-IL18 | FMC63 | YMNM |
| Arm B | 3^{rd} gen | pT4-CD19CARYSNV-IL18 | FMC63 | YMNM |
| Arm C | 2^{nd} gen | pT4-1928z-IL18 | SJ25C1 | YSNV |
| Arm D | 2^{nd} gen | pT4-1928YSNVz-18 | SJ25C1 | YSNV |

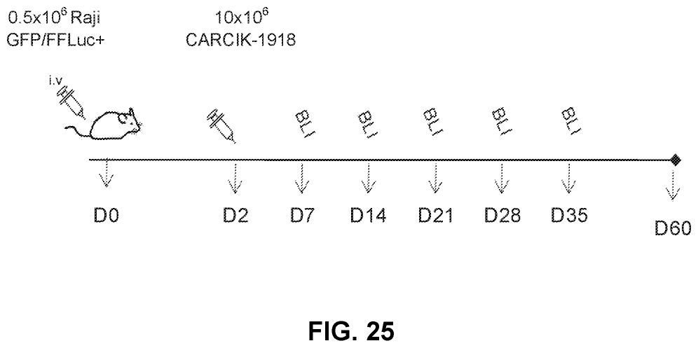

FIG. 25 shows a schematic representation of study design in NSG/Raji survival model.

### Raji Tumor Cell and Test System Preparation

GFP/FireFlyLuciferase (GFP/FFLuc) (construct described in Santos, E. B., et al. Nat. Med. 15(3):338-344 (2009)) positive Raji cells were thawed at least 1 week prior to the start of the in vivo experiment and seeded at a concentration of 0.5×10⁶ cells/mL in 5 mL RPMI supplemented with 10% Fetal Bovine Serum (FBS), 2 mM L-glutamine, 1% Penicillin/Streptomycin, 1% NEAA, 1 mM sodium pyruvate (Complete Medium). The cells were maintained twice weekly by splitting 2.5×10⁶ cells in 5 mL of fresh complete media (0.5×10⁶ cells/mL). When required, cells were expanded by determining cell count and increasing the culture volume to achieve 0.5×10⁶ cells/mL. The total number of cells seeded was the number of cells required to inject 0.5×10⁶ cells/animal on Day 0. The expanded cells were qualified prior to use for the presence of human CD19 and GFP by flow cytometry analysis using antibody specific for human CD19 and fluorescent emission consistent with GFP. The cells were qualified for luciferase by treating the cells with luciferin and measuring the bioluminescent output via a luminescence plate reader (Tecan).

Prior to Day 0 the supernatant from the expanded Raji cell line was tested for Mycoplasma using the Lonza MycoAlert kit (Lonza, LT07-318) according to the manufacturer's instructions.

On Day 0 mice were randomized to control and treatment groups. Raji cells were harvested, washed with PBS, counted, and formulated in PBS to a final concentration of 2.5×10⁶ viable cells/mL. Each mouse was injected intravenously (tail vein) with 200 μL of formulated GFP/FFLuc+ Raji cells (0.5×10⁶ cells/mouse) in a single administration. After two days, each lot comprising the four individual arms (Arm A, Arm B, Arm C, and Arm D as described in Table 15 above) of test articles were thawed, formulated, and infused into 5-8 mice per group with 10×10⁶ CARCIK-1918 cells/mouse. Six mice were left untreated. On Day 7 and weekly for 5 weeks of the study BLI measurements were taken to assess tumor burden.

NSG/Raji tumor mouse study outcome for testing of Lot A of CARCIK-1918 cells Arms A, B, C, and D

The key parameters for each arm of cells generated from Lot A are provided in Table 16. Lot A of CARCIK-1918 cells generated four sets of CARCIK-1918 cells Arms A, B, C and D with the indicated CAR transgene IL-18 bicistronic plasmid DNA (FIG. 24, Table 15).

TABLE 16
Characteristics of Lot A for each Arm of CARCIK-1918 cells
Lot ID A

| | Release | | | | |
|---|---|---|---|---|---|
| Attribute | Criteria | Arm A | Arm B | Arm C | Arm D |
| Viability (%) | ≥70 | 93 | 91 | 91 | 89 |
| CD3⁺ of total viable (%) | ≥90 | 99 | 99 | 99 | 99 |
| CD56⁺ of CD3 (%) | Report | 40.6 | 39.1 | 36.7 | 35.9 |
| CAR⁺ of CD3 (%) | ≥15 | 42.0 | 57.1 | 31.5 | 34.3 |
| IL-18 pg/mL | Report | 616 | 511 | 368 | 411 |
| Cytotoxicity (%) | ≥13 | 28 | 30 | 33 | 53 |
| VCN¹ (cp/mL) | Report | 1.61 | 2.40 | 9.80 | 9.09 |
| Sterility | No | No | No | No | No |
| | Growth | Growth | Growth | Growth | Growth |
| Endotoxin (EU/mL) | ≤6.25 | ≤1.25 | ≤1.25 | ≤1.25 | ≤1.25 |

¹VCN was tested on D 17 cells prior to formulation and freezing.

### Tumor Burden, Clinical Observations, and Survival

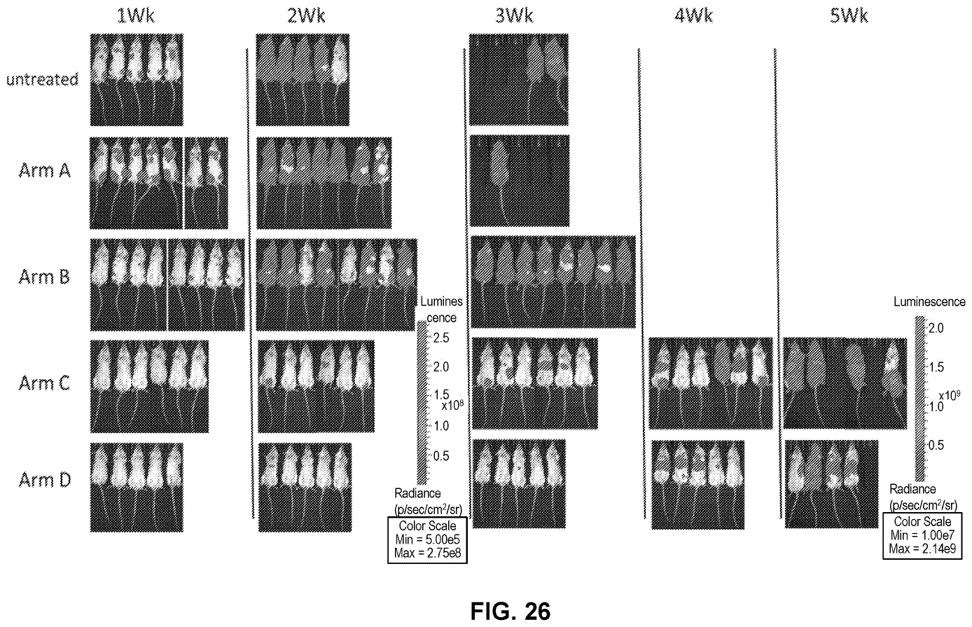

Tumor burden was assessed by bioluminescence imaging (BLI) of mice treated with CARCIK-1918 cells. (FIG. 26). Mice were either left untreated or treated with Arm A, B, C, and D CARCIK-1918 cells. BLI measurements were taken at weekly intervals, starting at week 1 and at weeks 2, 3, 4, and 5. At week 4 one animal in Arm D (#5 left to right) did not show tumor burden by BLI, but did not survive the anesthesia treatment. Therefore, 4 animals remained out to week 5 for analysis.

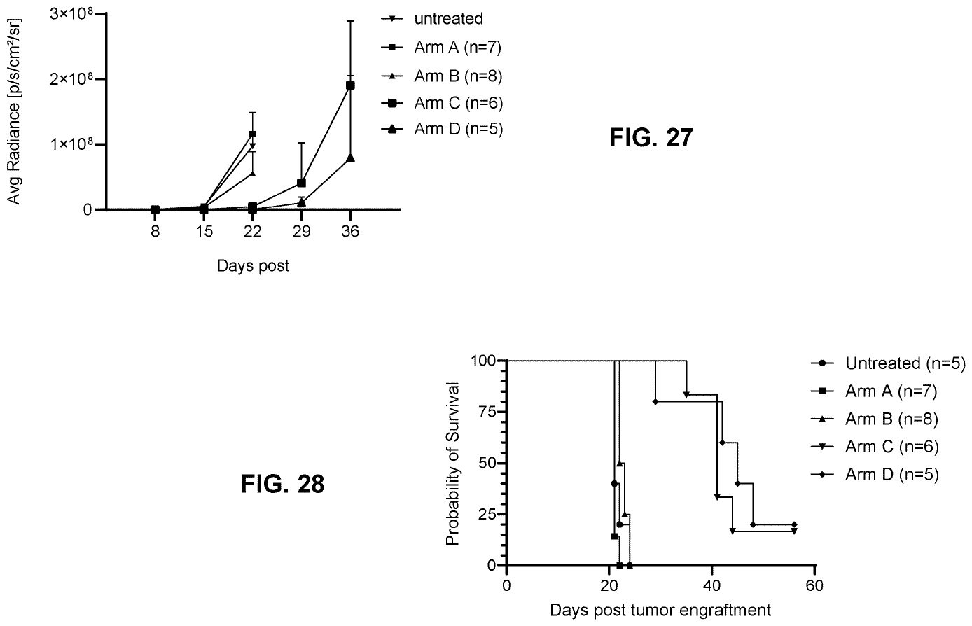

Region of interest (ROI) was recorded (FIG. 27) for each animal at weekly intervals starting at week one for five weeks. Animals in the untreated group and Arms A and B developed progressive highly disseminated tumor and succumbed to the disease by 3 weeks (days 21-23). Mice treated with Arm C and D CARCIK-1918 cells controlled tumor burden out to 5 weeks as demonstrated by decreased BLI signaling following CARCIK-1918 treatment. This is consistent with CARCIK-1918 derived anti-leukemic activity. There was a statistically significant decrease (p>0.01) in tumor burden for mice treated with either Arm C or Arm D 2^{nd} generation CAR transgene expressing CARCIK-1918 cells over either untreated animals or animals treated with Arm A and Arm B 3^{rd} generation CAR transgene expressing cells. By day 22 all animals in the untreated and 3^{rd} generation CAR transgene CARCIK-1918 cell treated groups succumbed to disease burden and were sacrificed according to the humane endpoint (FIG. 28). Both groups of mice treated with Arm C or Arm D cells exhibited similar control of tumor burden out to days 29 and 36. However, a lower incidence of tumor burden was observed in animals treated on Arm D with 2^{nd} generation CAR transgene CARCIK-1918 cells having the CD28 signaling attenuation compared to animals treated on Arm C (FIG. 27).

The ability to control tumor burden resulted in enhanced survival of the mice treated with cells from Arms C and D. Survival of these animals is summarized in the Kaplan-Meier survival plots in FIG. 28. All untreated mice developed progressive highly disseminated tumor and succumbed to disease after 22-24 days (n=5; median survival 21 days). There was no survival advantage in animals treated with CARCIK-1918 cells expressing a 3^{rd} generation CAR transgene. Median survival was 21 and 22.5 days for Arm A and Arm B respectively, which was not different from the untreated animals. However, CARCIK-1918 cells expressing the 2^{nd} generation CAR transgene had a statistically significant improvement in survival as compared to the untreated mice; Arm C (n=6; median survival 41 days; p=0.0015, log-rank test; 95% confidence interval (CI) 0.0507 to 1.159) and Arm D (n=5 median survival 45 days p-0.0015 log-rank test; 95% confidence interval (CI) 0.04541 to 1.082). No survival advantage was noted between animals treated with either Arm C or Arm D cells, CARCIK-1918 cells with or without the attenuated CD28 signaling domain.

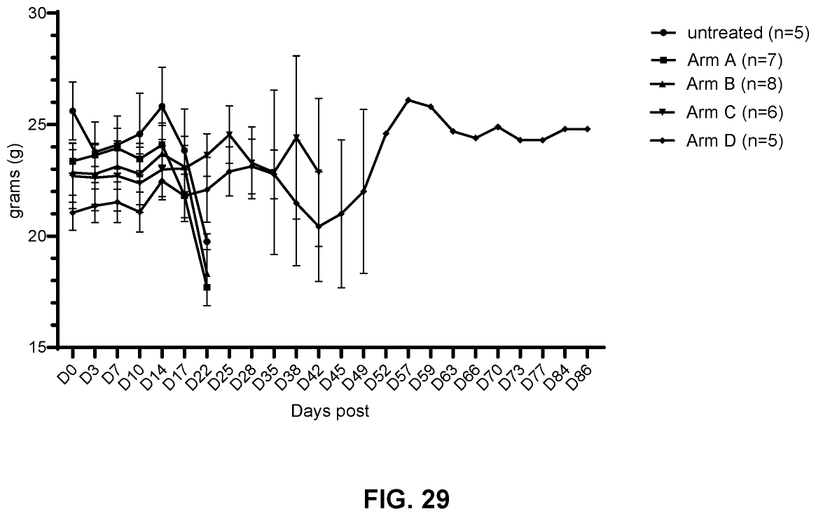

Individual animal body weight was measured and recorded twice weekly until animals succumbed to disease burned or reached the humane endpoint. The mean body weight for each experimental group was plotted versus sampling point for the duration of the study (FIG. 29). In the untreated animals and animals treated with CARCIK-1918 cells expressing a 3^{rd} generation CAR transgene, substantial weight loss, corresponding with animals succumbing to disease, was observed between Days 17 and 22, prior to animals reaching the humane endpoint. In contrast, animals treated with CARCIK cells expressing a 2^{nd} generation CAR transgene demonstrated steady body weight out to Day 42. There was no difference between groups of animals treated with 2^{nd} generation CAR transgene CARCIK-1918 cells containing the attenuated CD28 signaling domain and animals treated with CARCIK-1918 cells with the wildtype CD28 signaling domain. A single animal in Arm D survived out past 86 days before early signs of disease were noted. This animal was sacrificed on day 90, the humane endpoint for this animal.

### Measurement of CARCIK-1918 Cell Persistence in Peripheral Blood and Bone Marrow of Treated Animals

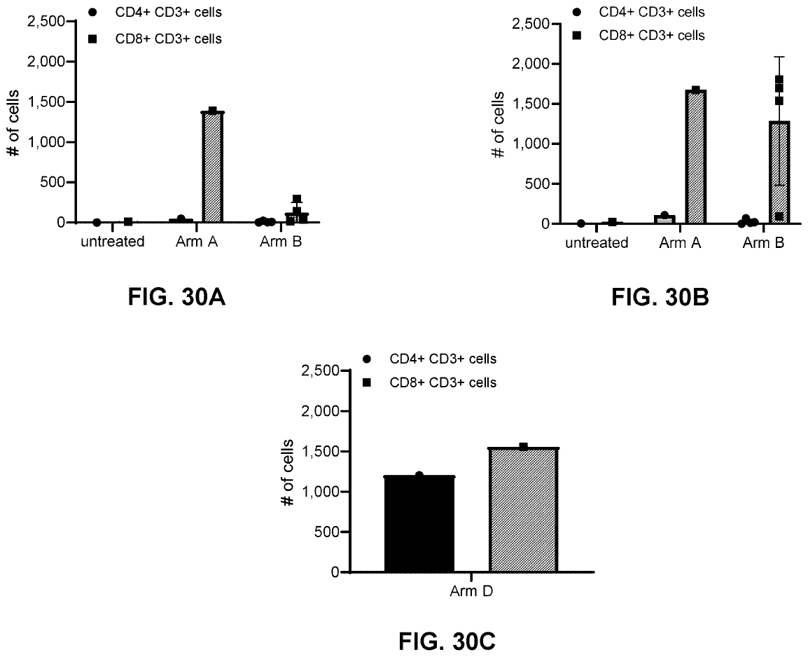

A limited number of animals from the untreated group or Arms A and B treated groups, where selected to measure CARCIK-CD1918 cell persistence by tracking the presence of viable human CD3+CD4+ and CD3+CD8+ cells by flow cytometry (FIG. 7A). Mice were sacrificed on day 22 due to disease progression and peripheral blood was collected. For Arm A, a single mouse analyzed had detectable numbers of CD3+CD8+ cells in the periphery (FIG. 30A). Low numbers of CD3+CD8+ cells were detected in Arm B animals (n=4) and no human CD3+CD4+ or CD3+CD8 cells were detected in the single untreated animal analyzed (FIG. 30A). Bone marrow was also collected at the day 22 timepoint for the same set of animals. Human CD3+CD8+ cells could be detected in the bone marrow from a single animal treated on Arm A and 3 of the 4 animals treated on Arm B (FIG. 30B). However, the presence of human CD3+CD4 or CD3+CD8+ cells in the bone marrow of mice treated on Arms A or B with the 3rd generation expressing CAR transgene CARCIK-1918 cells did not correlate with survival nor control of tumor burden (FIGS. 26, 27, and 28). All animals regardless of the presence of 3rd generation CARCIK-1918 cells succumbed to disease at a similar rate to the untreated animals (FIG. 27). A single animal from Arm D was analyzed at day 46, the time of humane endpoint sacrifice, and the presence of both CD3+CD4+ and CD3+CD8+ cells were detected in the peripheral blood (FIG. 30C), which suggests that that attenuation of the CD28 signaling domain in the 2^{nd} generation CAR transgene results in stronger control of tumor burden.

Cytokine Profile in Mice Treated with CARCIK-1918 Cells

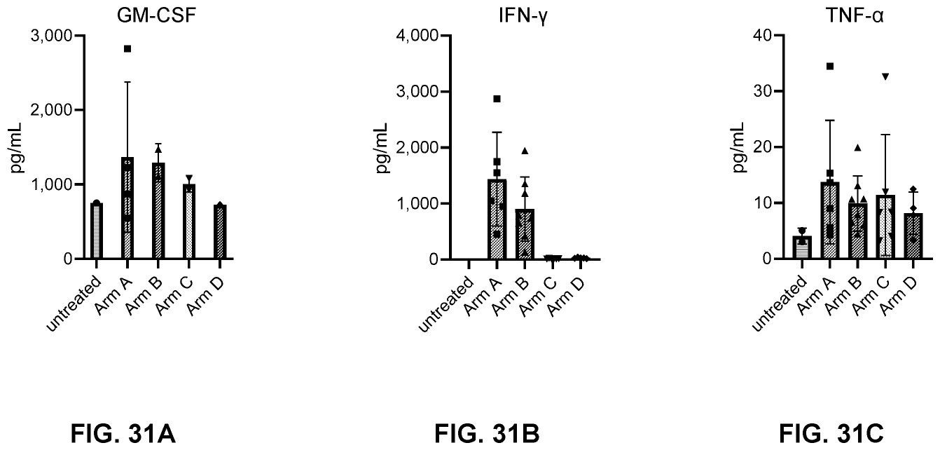

Circulating levels of GM-CSF (FIG. 31A), IFN-γ (FIG. 31B), TNF-α (FIG. 31C) and IL-18 (data not shown) were measured in the untreated animals or animals in the CARCIK-1918 cell treated groups. Baseline levels of 750 μg/mL of GM-CSF were measured in a single untreated mouse. Animals treated on Arm A (n=4) and Arm B (n=2) with 3^{rd} generation CARCIK-1918 cells had slightly higher levels of GM-CSF compared to animals on Arm C (n=2) and Arm D (n=1) treated with 2^{nd} generation CARCIK-1918 cells. Mice treated on Arm A (n=6) and Arm B (n=8) with 3^{rd} generation CARCIK-1918 cells had the highest levels of IFN-γ compared to animal treated on Arm C (n=6) and Arm D (n=5) with 2^{nd} generation CARICK-1918 cells. While no untreated animals were sampled to establish in vivo baseline human IFN-γ levels, it is unlikely that mice will produce human IFN-γ. Low levels (less than 40 μg/mL) of TNF-α were seen in all treated animals. No differences were noted between any of the treated groups. While CARCIK-1918 cells could be stimulated to secrete IL-18 in vitro with CD19+ REH tumor cells, no IL-18 was detected above the limitation of detection for the assay in the untreated nor in any animals treated with CARCIK-1918 cells on any of the experimental Arms (data not shown).

NSG/Raji tumor mouse study outcome for testing of Lot B of CARCIK-1918 cells Arms A, B, C, and D

The key parameters for each arm of cells generated from Lot B are provided in Table 17. Lot A of CARCIK-1918 cells generated four sets of CARCIK-1918 cells Arms A, B, C and D with the indicated CAR transgene IL-18 bicistronic plasmid DNA (FIG. 24, Table 15).

TABLE 17
Characteristics of Lot B for each arm of CARCIK-1918 cells
Lot ID B

| | Release | | | | |
|---|---|---|---|---|---|
| Attribute | Criteria | Arm A | Arm B | Arm C | Arm D |
| Viability (%) | ≥70 | 91 | 91 | 92 | 91 |
| CD3⁺ of total viable (%) | ≥90 | 99 | 99 | 99 | 99 |
| CD56⁺ of CD3 (%) | Report | 33 | 38 | 30 | 34 |
| CAR⁺ of CD3 (%) | ≥15 | 50 | 69 | 32 | 18 |
| IL-18 pg/mL | Report | 516 | 683 | 260 | 107 |
| Cytotoxicity (%) | ≥13 | 52 | 65 | 60 | 47 |
| VCN¹ (cp/mL) | Report | 2.7 | 3.8 | 5.9 | 2.7 |
| Sterility | No | No | No | No | No |
| | Growth | Growth | Growth | Growth | Growth |
| Endotoxin (EU/mL) | ≤6.25 | ≤1.00 | ≤1.00 | ≤1.00 | ≤1.00 |
| Mycoplasma | Absent | Absent | Absent | Absent | absent |

¹VCN was tested on D 17 cells prior to formulation and freezing.

### Tumor Burden, Clinical Observations, and Survival

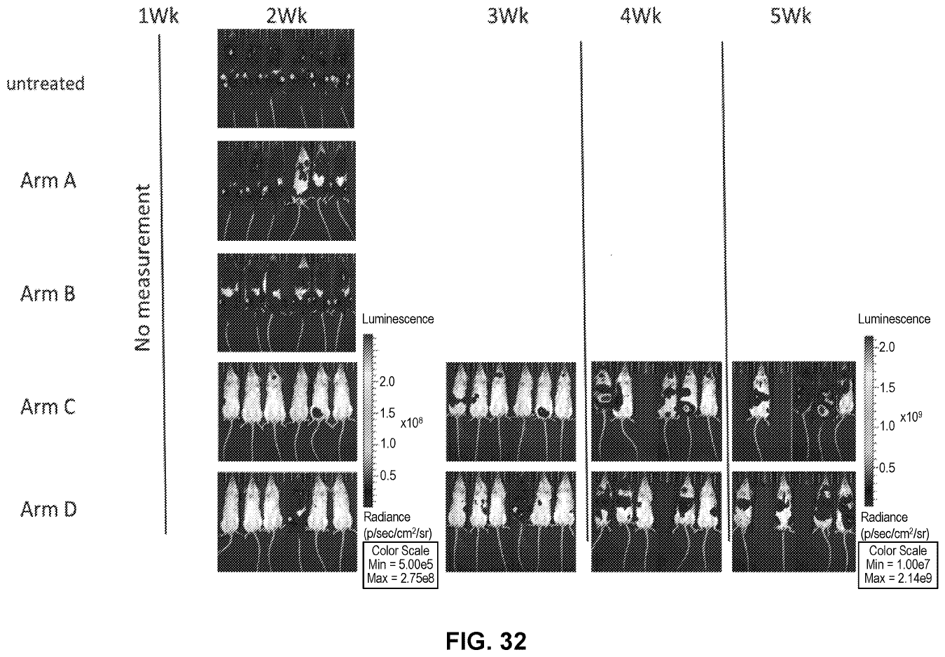

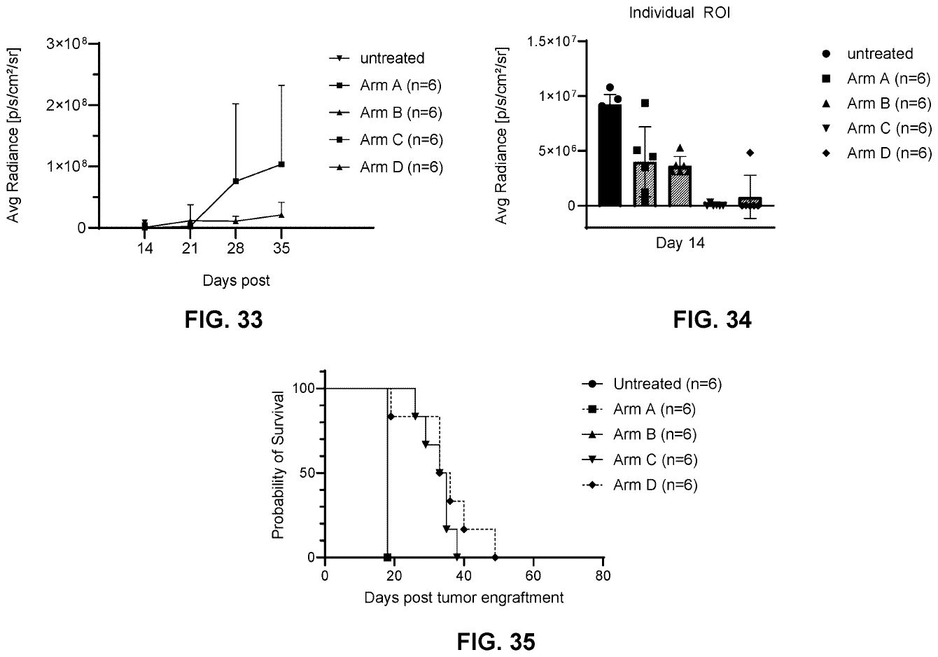

Tumor burden was assessed by BLI (FIG. 32), and region of interest (ROI) was recorded for each animal at weekly intervals starting at week two for five weeks (FIG. 33). Animals in the untreated group and Arms A and B developed progressive highly disseminated tumor and succumbed to disease by day 18 (FIG. 35). To assess early tumor burden control in the absence of a reading on day 7 (week 1) individual ROI was plotted for each animal in groups, untreated, Arms A, B, C and D at day 14 (FIG. 34). On day 14 the greatest decrease in tumor growth was observed for mice treated with CARCIK-1918 cells containing the 2^{nd} generation CAR transgene (either Arms C and D) compared to the untreated mice and mice treated with CARCIK-1918 cells containing the 3^{rd} generation CAR transgene (Arms A and B). This decrease observed on day 14 was statistically significant (p<0.01). Mice treated with Arms C and D CARCIK-1918 cells were capable of controlling tumor burden out to 5 weeks as demonstrated by decreased BLI signaling following CARCIK-1918 treatment, which is consistent with CARCIK-1918 derived anti-leukemic activity (FIG. 32). Moreover, a considerable decrease in tumor burden was noted in animals treated on Arm D compared to animals treated on Arm C (FIG. 33), which suggests that that attenuation of the CD28 signaling domain in the 2^{nd} generation CAR transgene results in stronger control of tumor burden.

The ability of CARCIK-1918 cells expressing the 2^{nd} gen CAR transgene to control tumor burden resulted in enhanced survival of mice. A similar survival benefit was observed in mice treated with cells from either Arms C or D. Survival is summarized in the Kaplan-Meier survival plots in FIG. 35. All untreated mice developed progressive, highly disseminated tumor, and succumbed to disease after 18 days (n=6; median survival 18 days). There was no survival advantage in animals treated with CARCIK-1918 cells expressing the 3^{rd} generation CAR transgene over the untreated animals (median survival was 18 days for animals treated with Arm A and B cells). However, CARCIK-1918 cells expressing the 2^{nd} generation CAR transgene had a statistically significant improvement in survival as compared to untreated mice; Arm C (n=6; median survival 34 days; p=0.0009, log-rank test; 95% confidence interval (CI) 0.0418 to 1.086) and Arm D (n=6 median survival 34.5 days p=0.0009 log-rank test; 95% confidence interval (CI) 0.0902 to 1.231). No difference in survival was seen between animals treated with the CARCIK-1918 cells containing the attenuated CD28 signaling domain and those with the wildtype domain.

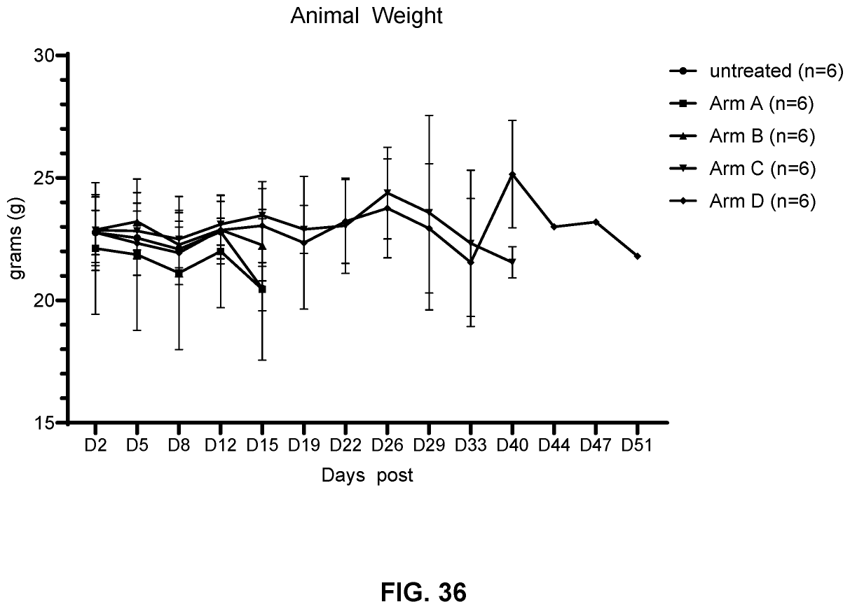

Individual mouse body weight was recorded twice weekly for all animals on study until animals succumbed to disease burden or reached the humane end point. The mean body weight for each experimental group was plotted versus sampling point for the duration of the study (FIG. 36). In the untreated animals and animals treated with CARCIK-1918 cells expressing a 3^{rd} generation CAR transgene, substantial weight loss, corresponding with animals succumbing to disease, was observed by day 15, prior to animals reaching the humane endpoint. In contrast, animals treated with CARCIK-1918 cells expressing the 2^{nd} generation CAR transgene demonstrated steady body weight out to Day 40 or day 51 until animals succumbed to disease and reached the humane endpoint. There were no differences in body weight between groups of animals treated with 2^{nd} generation CAR transgene CARCIK-1918 cells containing the attenuated CD28 signaling domain and animals treated with CARCIK-1918 cells with the wildtype CD28 signaling domain.

### Measurement of Leukemic Burden in the Peripheral Blood of Treated Animals

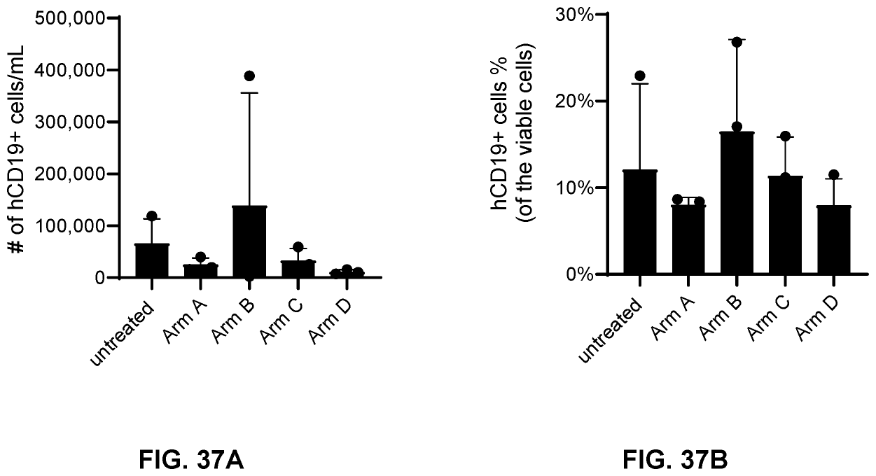

Peripheral blood was collected by retro-orbital (RO) bleed from untreated, and mice treated with each Arm of CARCIK-1918 cells (n=3 for each group) on day 8. The presence of circulating tumor cells was measured by flow cytometry by staining viable cells for human CD19 expressed on circulating Raji tumor cells (hCD19+ cells). Lower numbers of circulating tumor cells were detected in all treated groups compared to the untreated animals, except for a single animal in Arm B having high tumor burden (FIG. 37A). Furthermore, the lowest number of circulating tumor cells was observed in mice (n=3) treated on Arm D. A similar pattern of reduction in the frequency of circulating tumor cells was observed for all three mice treated with CARCIK-1918 cells on Arm D (FIG. 37B).

### Measurement of CARCIK-1918 Cell Persistence in Peripheral Blood of Treated Animals

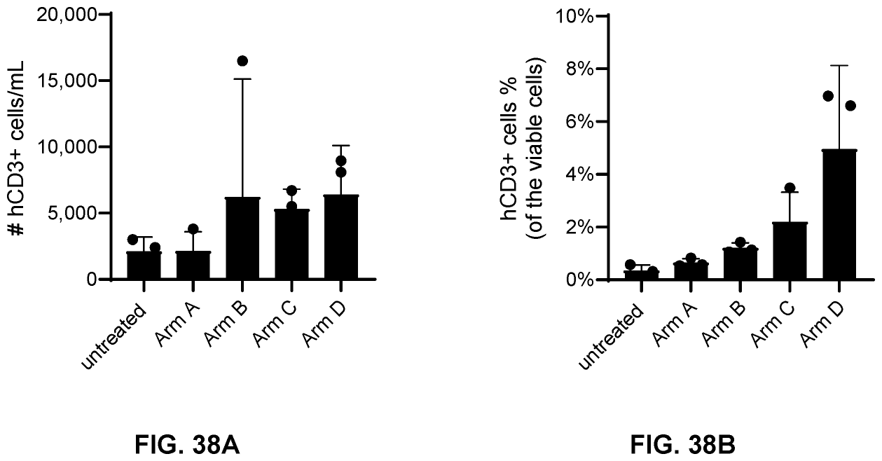

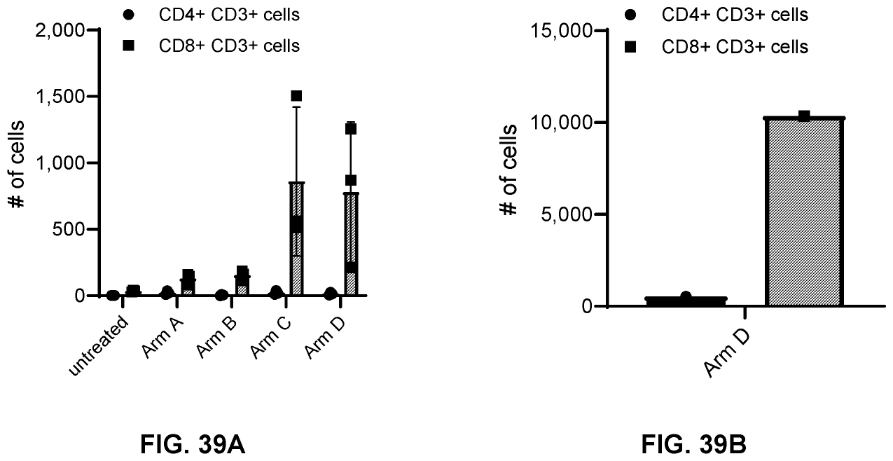

Peripheral blood from untreated mice or mice treated with each Arm of CARCIK-1918 cells was collected by RO bleeding on day 8. The persistence of CARCIK-1918 cells in the peripheral blood was measured with flow cytometry by staining viable cells for the human CD3 marker expressed on the infused CARCIK-1918 cells (hCD3+ cells). Greater numbers of CARCIK-1918 cells were seen in mice treated with Arms C and D CARCIK-1918 cells compared to untreated animals or animals treated with Arms A and B CARCIK-1918 cells (FIG. 38A). Only one of three mice on Arm B did exhibit high numbers of circulating CARCIK-1918 cells (FIG. 38A). Mice treated with Arm Cor Arm D CARCIK-1918 cells showed the highest frequency of CARCIK-1918 cells (FIG. 38B), with 2 of 3 mice treated with CARCIK-1918 cells with the attenuated CD28 signaling domain having the highest frequency of CARCIK-1918 cells. Further breakout of hCD3+ cells by either CD4+ or CD8 positivity revealed the hCD3+ cell population to be predominantly composed of CD8+ cells (FIG. 39A) for animals treated on Arms C and D. A single mouse treated with Arm D CARCIK-1918 cells containing the attenuated CD28 signaling domain when sacrificed at the humane endpoint at day 40 showed that CD8+CD3+ CARCIK-1918 cells were still present in the periphery (FIG. 39B).

Cytokine Profile in Mice Treated with CARCIK-1918 Cells

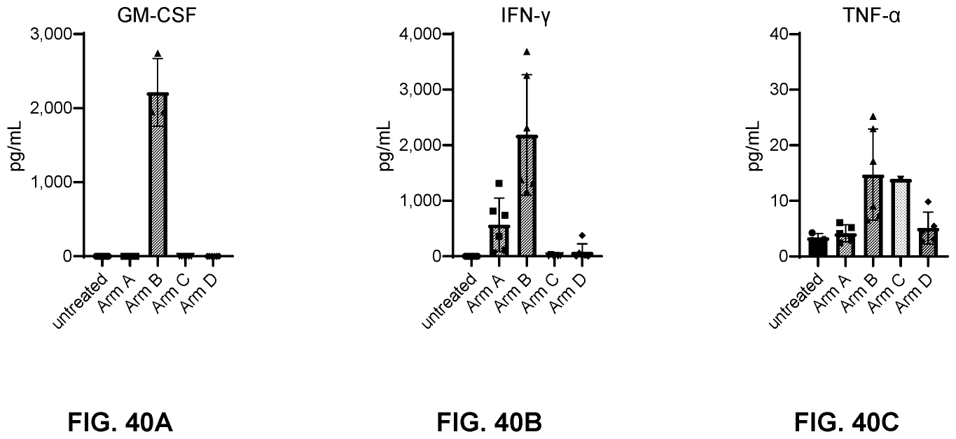

As an exploratory analysis circulating levels of GM-CSF (FIG. 40A), IFN-γ (FIG. 40B), TNF-α (FIG. 40C) and IL-18 (data not shown) were measured in the untreated animals or animals in the CARCIK-1918 cell treated groups. GM-CSF was not detected above the assay limit of detection for untreated animals or animals treated on Arms A, C, and D. However, GM-CSF in the range of 2,000 μg/mL was detected for three of the animals treated on Arm B. Moreover, higher levels of IFN-γ were detected in animals treated on Arms A and B with the 3^{rd} generation CARCIK-1918 cells, with Arm B treated mice having the highest recorded levels, suggesting that attenuation of the CD28 signaling domain may lead to higher IFN-γ secretion with a 3^{rd} generation CAR transgene. Low levels (less than 40 μg/mL) of TNF-α were seen in all treated animals. Animals treated on Arms B and C had the highest levels with Arms A and D having levels comparable to the untreated animals. While CARCIK-1918 cells could be stimulated to secrete IL-18 in vitro with CD19+ REH tumor cells, no IL-18 was detected above the limitation of detection for the assay in the untreated or in any animals treated with CARCIK-1918 cells on any of the experimental Arms (data not shown).

### Example 9

### In Vivo Evaluation of CARCIK Cells in CD19+ Human Burkitt's Lymphoma Mouse Model (NSG/Daudi)

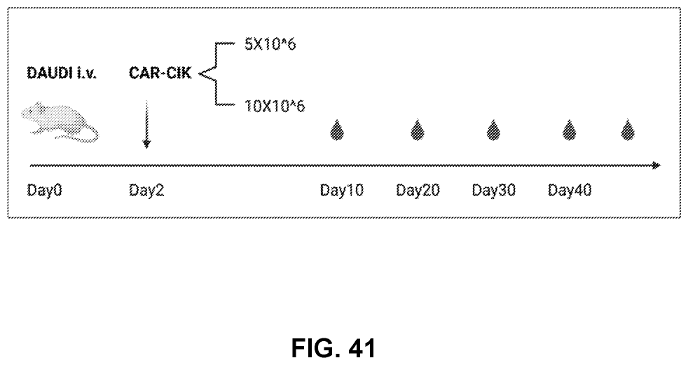

The anti-tumor efficacy of CARCIK-1918 cells was evaluated in Daudi cell tumor bearing NOD scid gamma (NSG) immunocompromised mice. FIG. 41 shows a schematic representation of study design in NSG/Daudi survival model.

### Daudi Tumor Cell and Test System Preparation

Daudi cells were thawed 10 days prior to the start of the in vivo experiment and seeded at a concentration of 1×10⁶ cells/mL in RPMI 1640 medium supplemented with 10% Fetal Bovine Serum (FBS), 1% L-glutamine and Penicillin/Streptomycin (Complete Medium). The cells were expanded by passage twice weekly to achieve at least 0.2×10⁵ cells/animal on Day 0. The expanded cells were qualified prior to use for the presence of human CD19 by flow cytometry analysis using antibody specific for human CD19. Briefly, 100,000 cells were stained with 2 μL of antibody in Phosphate Buffered Saline (PBS) supplemented with 2% FBS and acquired on the BD FACS Canto II.

Prior to Day 0 the supernatant from the expanded Daudi cell line was tested for Mycoplasma using the MycoBlue Mycoplasma Detector D101 according to the manufacturer's instructions. On Day 0 mice were randomized to control and treatment groups. Daudi cells were harvested, washed with PBS, counted, and formulated in PBS to a final concentration of 1×10⁵ viable cells/mL. A total of 17 (6-9-week-old) NSG mice (Charles River Laboratories, Calco, Italy) were injected with Daudi cells on Day 0 of the study to establish the test system. Each mouse was injected intravenously (tail vein) with 200 μL of formulated Daudi cells (0.2×10⁵ cells/mouse) in a single administration. After two days, a single lot of test article was thawed, formulated, and injected at two dose levels into 6 mice per level (5×10\hat{ }6 and 10×10\hat{ }6 CARCIK-1918 cells/mouse). The remaining 5 mice were left untreated. On Day 10 of the study and every 10 days thereafter peripheral blood was collected from the mice in each experimental group for flow cytometric analysis.

CARCIK-1918 cells were generated with the pT4-1928YSNVz-IL18 (FIG. 24, plasmid D) donor transposon and evaluated in Daudi cell tumor bearing NSG mice.

The key parameters for CARCIK-1918 cells generated from Lot C are provided in Table 18.

TABLE 18
Characteristics of Lot C CARCIK-1918 cells
Lot ID C

| Attribute | Release Criteria | Result |
|---|---|---|
| Viability (%) | ≥70 | 87 |
| CD3 of total viable (%) | ≥90 | 96 |
| CD56 of CD3 (%) | Report | 86 |
| CAR of CD3 (%) | ≥15 | 48 |
| Cytotoxicity (%) | ≥13 | 71 |
| IL-18 (pg/mL) | Report | 213.42 |
| VCN (cp/cell) | Report | 10.0¹ |
| Sterility | No Growth | No Growth |
| Endotoxin (EU/mL) | ≤6.25 | <1.00 |
| Mycoplasma | Absent | Absent |

¹VCN was tested on D 17 cells prior to formulation and freezing

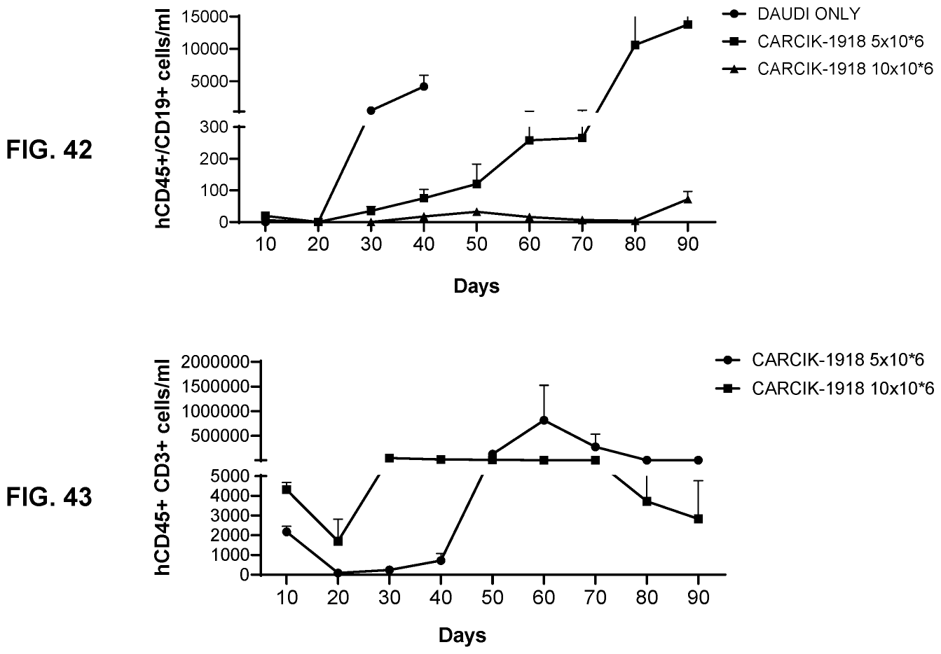

The mean concentration of hCD45+/hCD19+ cells/mL of blood for each experimental group (DAUDI only, CARCIK-1918 5×10\hat{ }6, CARCIK-1918 10×10\hat{ }6) was plotted versus sampling point to monitor cell expansion over the course of the study (FIG. 42). The leukemic cells expanded in the untreated group to a peak around Day 40 after which the animals succumbed to disease or were sacrificed. Control of disease was observed in the treated groups as indicated by the low mean concentration of circulating Daudi cells over the course of the study, particularly at the 10×10⁶ dose level.

The mean concentration of hCD45+/hCD3+ cells for each experimental group (CARCIK-1918 5×10\hat{ }6, CARCIK-1918 10×10\hat{ }6) was plotted versus sampling point to monitor cell expansion over the course of the study (FIG. 43). CARCIK-1918 cells were detected in the peripheral blood of mice treated with both dose levels over the entire course of the study. At the 10×10⁶ dose level, the mean number of hCD45+/hCD3+ cells/mL in the blood peaked around Day 30 and maintained a consistent level until Day 70 before beginning to decline. This consistently high level of CARCIK-1918 engraftment is correlated with the maintenance of a consistently low level of hCD19+ leukemic cell detection over the same period (FIG. 42).

CARCIK-1918 cells in the animals infused with the 5×10⁶ dose level were also detected at each measurement but at a lower level than in the animals infused with the higher dose until Day 40 (FIG. 43). The mean number of CARCIK-1918 cells/mL in the blood in the lower dose treatment group began to increase after Day 40, reaching peak detection at Day 60.

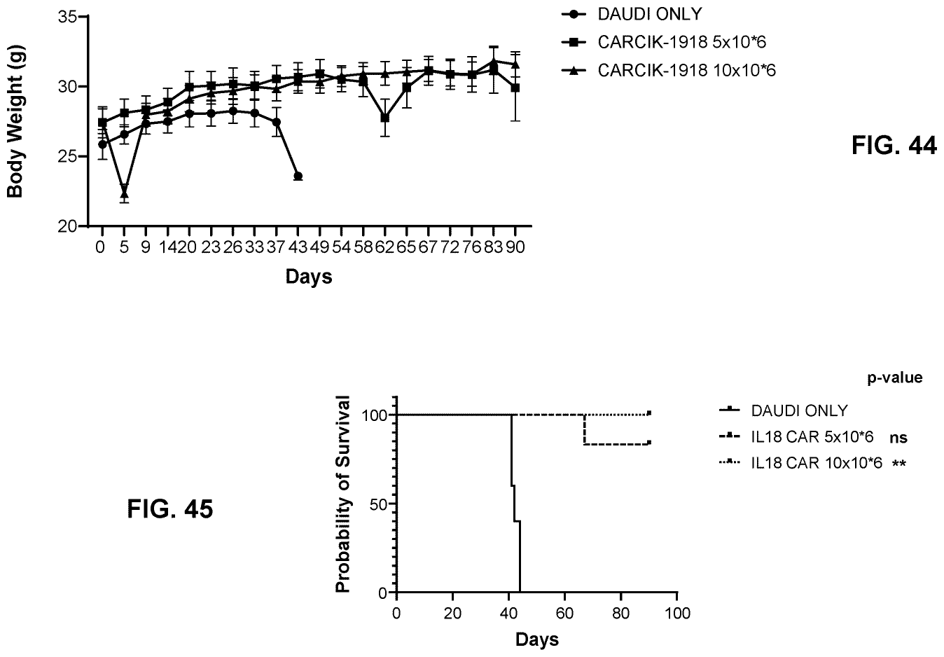

Individual mouse body weights were measured and recorded at least twice weekly until the animals succumbed to disease or reached the humane endpoint. The mean body weight for each experimental group was plotted versus sampling point for the duration of the study (FIG. 44). In the untreated animals, substantial weight loss, corresponding with the peak detection of circulating tumor cells, was observed between Days 30 and 40 just prior to the humane endpoint for this experimental group. In contrast, the animals treated with 5×10⁶ CARCIK-1918 cells demonstrated a steady increase in body weight out to Day 50 of the study after which a substantial decline in weight between Days 58 and 62 was observed, corresponding to the peak detection of circulating hCD45+/hCD19+ leukemic cells (FIG. 42). Except for a noticeable and transient weight decline over the first 5 days of the study, which fully recovered by Day 10, the animals treated with 10×10⁶ CARCIK-1918 cells exhibited a steady increase in weight over the entire study consistent with control of disease as corroborated by the low level of detected circulating tumor cells (FIG. 42).

All the untreated mice were dead or at the humane endpoint by Day 44 of the study and had exhibited signs of disease burden including ruffled fur, closed eyes, hunched back and paralysis in both limbs (Table 19). Of the 6 mice treated with 5×10⁶ CARCIK-1918 cells, 5 were still alive at Day 90 and only 1 of the surviving 5 was exhibiting clinical signs of disease burden. All 6 of the mice treated with 10×10⁶ CARCIK-1918 cells were still alive on Day 90 and none of these 6 were displaying signs of suffering or paralysis.

TABLE 19
Summary of Clinical Observations

| | | Death, | | | | |
|---|---|---|---|---|---|---|
| Experimental | Animal | Study | | Descriptive | Paralysis | Weight |
| Group | Number | Day | Notes | Evaluation | Grade | Grade |
| Untreated | N | 40 | | R/O/H | 3 | 1 |
| Daudi Only | L | 42 | Found Dead | R/O/H | 3 | 1 |
| | R | 40 | | R/O/H | 3 | 1 |
| | LL | 44 | | R/O/H | 3 | 1 |
| | RR | 44 | | R/O/H | 3 | 3 |
| Treated | N | 90 | | | 0 | 0 |
| 5 × 106 | L | 90 | | | 0 | 0 |
| CARCIK- | R | 90 | | | 0 | 0 |
| 1918 cells | LL | 68 | Found Dead | R/O/H | 2 | 3 |
| | RR | 90 | | | 0 | 0 |
| | LR | 90 | | R | 0 | 2 |
| Treated | N | 90 | | | 0 | 0 |
| 10 × 106 | L | 90 | | | 0 | 0 |
| CARCIK- | R | 90 | | | 0 | 0 |
| 1918 cells | LL | 90 | | | 0 | 0 |
| | RR | 90 | | | 0 | 0 |
| | LR | 90 | | | 0 | 0 |

Clinical description abbreviations:
Ruffled Fur (R),
Tachypnea (T),
Chromodacryorrhoea (C),
Closed eyes (O),
Hunched back (H),
Stress (S)

### Survival Analysis

All untreated mice developed progressive highly disseminated tumor and succumbed to the disease after 41-44 days (n=5) (FIG. 45). CARCIK-1918 cells at both dose levels demonstrated an inhibitory effect on disease progression. Five of the 6 mice treated at the 5×10⁶ CARCIK-1918 dose level and all 6 of the 6 mice treated at the 10×10\hat{ }6 level were still alive at the study termination on day 90 (FIG. 45). A statistically significant improvement in survival relative to the untreated control group was observed only at the 10×10⁶ CARCIK-1918 dose level (p-value 0,0011, Log-rank Mantel Cox test). Lack of statistical significance in the survival difference between the untreated (Daudi only) group and the group treated with 5×10⁶ CARCIK-1918 cells is likely due to the small number of animals evaluated. Nevertheless, a substantial qualitative improvement in survival for mice treated at the low dose level was readily observed.

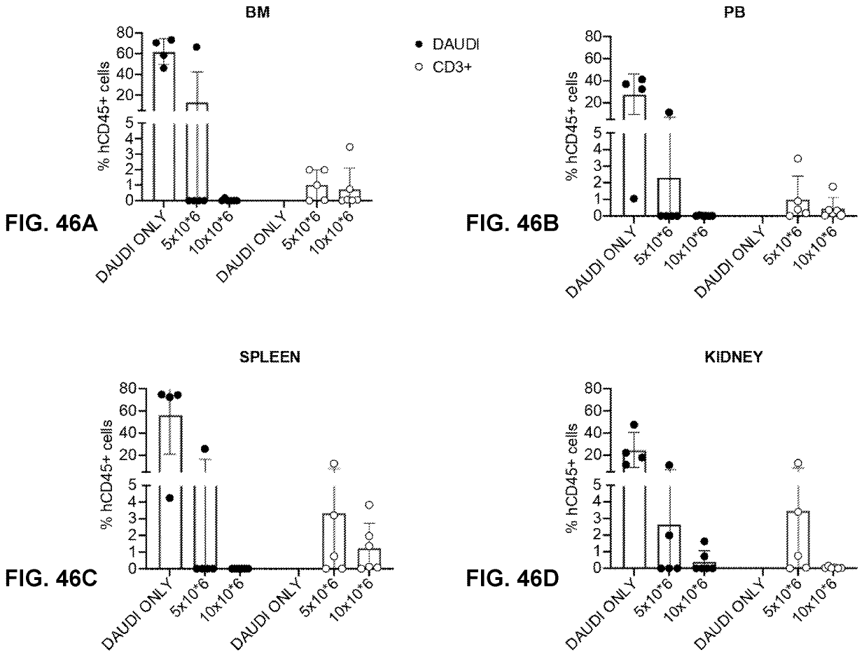

The post-mortem analysis of peripheral blood and tissues demonstrated that CARCIK-1918 cells from both dose levels persisted over time and limited the level of leukemic cell dissemination in the animals. Notably, the 10×10⁶ regimen allowed an almost total control of the disease progression in all tissues examined (in the bone marrow (BM) (FIG. 46A), peripheral blood (PB) (FIG. 46B), spleen (FIG. 46C), and kidney (FIG. 46D)).

1. A method of generating genetically modified cytokine induced killer (CIK) cells, comprising the sequential steps of:
(a) culturing a population of mononuclear cells in culture medium comprising at least one differentiating agent to induce differentiation of the mononuclear cells in a cell culture into Committed CIK Precursor cells;
(b) adding at least one stimulating agent and at least one expanding agent to the cell culture; and
(c) expanding the cells from the cell culture to obtain a cell population comprising Committed CIK Precursor cells, transfecting the cell population comprising the Committed CIK Precursor cells with one or more nucleic acids to produce genetically modified Committed CIK Precursor cells, and expanding the genetically modified Committed CIK Precursor cells in culture medium to produce the genetically modified CIK cells; or (c) expanding the cells from the cell culture to obtain a cell population comprising Committed CIK Precursor cells and transfecting the cell population comprising the Committed CIK Precursor cells with one or more nucleic acids;
wherein steps (a), (b) and (c) are performed in the absence of non-irradiated or irradiated feeder cells.

2. The method of claim 1, further comprising
(d) formulating transfected cells comprising the Committed CIK Precursor cells for administration to a subject in need thereof; and
(e) administering the transfected cells to the subject, wherein transfected Committed CIK Precursor cells are expanded to produce the genetically modified CIK cells in the subject.

3. A method of generating genetically modified cytokine induced killer (CIK) cells, comprising the sequential steps of:
(a) culturing peripheral blood mononuclear cells (PBMCs) in culture medium comprising at least one differentiating agent to induce differentiation of the PBMCs in a cell culture into Committed CIK Precursor cells;
(b) adding at least one stimulating agent and at least one expanding agent to the cell culture; and
(c) expanding the cells from the cell culture to obtain a cell population comprising Committed CIK Precursor cells, transfecting the cell population comprising the Committed CIK Precursor cells with one or more nucleic acids to produce genetically modified Committed CIK Precursor cells, and expanding the genetically modified Committed CIK Precursor cells in culture medium to produce the genetically modified CIK cells; or (c) expanding the cells from the cell culture to obtain a cell population comprising Committed CIK Precursor cells and transfecting the cell population comprising the Committed CIK Precursor cells with one or more nucleic acids;
wherein steps (a), (b) and (c) are performed in the absence of non-irradiated or irradiated feeder cells; and
wherein the PBMCs were previously cryopreserved and thawed before culturing in step (a).

4. The method of claim 2, further comprising
(d) formulating transfected cells comprising the Committed CIK Precursor cells for administration to a subject in need thereof; and
(e) administering the transfected cells to the subject, wherein transfected Committed CIK Precursor cells are expanded to produce the genetically modified CIK cells in the subject.

5. The method of claim 1, wherein the stimulating agent and the expanding agent are added to the cell culture approximately 8 to 48 hours after initiating step (a),
wherein the Committed CIK Precursor cells are transfected with one or more nucleic acids approximately 18 to 48 hours after initiating step (b), and/or
wherein the method further comprises (d) replacing a portion of the culture medium with a fresh culture medium comprising at least one expanding agent approximately 8 to 96 hours after initiating step (c).

6. The method of claim 5, wherein the stimulating agent and the expanding agent are added to the cell culture approximately 18 to 24 hours after initiating step (a).

7. (canceled)

8. The method of claim 57, wherein the Committed CIK Precursor cells are transfected with one or more nucleic acids approximately 18 to 24 hours after initiating step (b).

9. (canceled)

10. The method of claim 59, further comprising (d) replacing a portion of the culture medium with a fresh culture medium comprising at least one expanding agent approximately 18 to 24 hours after initiating step (c), and/or further comprising (e) passaging and culturing transfected cells in culture medium comprising at least one expanding agent approximately 8 to 96 hours after initiating step (d).

11. (canceled)

12. The method of claim 10, further comprising (e) passaging and culturing transfected cells in culture medium comprising at least one expanding agent approximately 72 to 96 hours after initiating step (d).

13-21. (canceled)

22. The method of claim 12, further comprising (f) isolating the cells from the cell culture to obtain a cell population comprising the genetically modified CIK cells, or wherein the transfected cells are cultured to obtain a cell population comprising the genetically modified CIK cells.

23-24. (canceled)

25. The method of claim 1, wherein the genetically modified CIK cells express one or more T cell receptors (TCR-CIK), chimeric antigen receptors (CAR-CIK), a genetically modified cell adhesion molecule, a genetically modified ligand, a genetically modified cytokine receptor, a genetically modified chemokine receptor, a genetically modified cytokine, a genetically modified chemokine, an enzyme, or a checkpoint inhibitor, or wherein the genetically modified CIK cells express a prodrug converting enzyme, an IgG-degrading enzyme of S. pyogenes (IdeS), a consensus variant 1 (CV1) protein, Intercellular Adhesion Molecule 1 (ICAM-1), CD137L, OX40L, CD70, IL-15 receptor, CCR5, CCR4, CD25, CD122, CD132, C-X-C chemokine receptor type 4 (CXCR4), IL-15, IL-18, IL-21 IL-23 IL-33, IL-1a, IL-1b, matrix metalloproteinase (MMP), heparinase, an anti-PD-1 antibody or antigen binding fragment thereof, an anti-T cell immunoglobulin and mucin-domain containing-3 (TIM-3) antibody or antigen binding fragment thereof, IL-3 zetakine, or any combination thereof.

26. (canceled)

27. The method of claim 1, wherein the population of mononuclear cells is selected from the group consisting of: umbilical cord blood derived mononuclear cells, peripheral blood mononuclear cells (PBMCs), bone marrow derived mononuclear cells, lymphocytes, monocytes, dendritic cells, macrophages, T cells, naive T cells, memory T cells, natural killer cells, hematopoietic stem cells, pluripotent embryonic stem cells, induced pluripotent stem cells, and any combination thereof.

28. The method of claim 27, wherein the mononuclear cells are umbilical cord blood derived mononuclear cells, or PBMCs, and/or wherein the mononuclear cells are from a human leukocyte antigen (HLA) matched donor.

29-31. (canceled)

32. The method of claim 1, wherein the differentiating agent is selected from the group consisting of: IFN-γ, IL-4, IL-5, IL-7, IFN-α, IL-10, IL-12, IL-13, IL-6, IL-15, IL-17, IL-18, IL-21, IL-22, IL-23, IL-27, IL-1β, TGF-β, GM-CSF, CCL3, CCL4, CCL5, CCL17, CCL21, and any combination thereof, and/or wherein the differentiating agent is added in an amount of about 10 U/ml to about 10000 U/ml.

33-35. (canceled)

36. The method of claim 1, wherein the stimulating agent is selected from the group consisting of: an anti-CD3 antibody, an anti-TCR antibody, an anti-CD28 antibody, an anti-CD137 antibody, an anti-CD134 antibody, an anti-CD27 antibody, an anti-ICAM-1 antibody, an anti-CD3/CD28-coated beads, a superantigen, phytohaemaglutinin (PHA), phorbol 12-myristate 13-acetate (PMA), ionomycin, and any combination thereof, and/or wherein the stimulating agent is added in an amount of about 5 ng/ml, about 6 ng/ml, about 7 ng/ml, about 8 ng/ml, about 9 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 31 ng/ml, about 32 ng/ml, about 33 ng/ml, about 34 ng/ml, about 35 ng/ml, about 36 ng/ml, about 37 ng/ml, about 38 ng/ml, about 39 ng/ml, about 40 ng/ml, about 41 ng/ml, about 42 ng/ml, about 43 ng/ml, about 44 ng/ml, about 45 ng/ml, about 46 ng/ml, about 47 ng/ml, about 48 ng/ml, about 49 ng/ml, about 50 ng/ml, about 51 ng/ml, about 52 ng/ml, about 53 ng/ml, about 54 ng/ml, about 55 ng/ml, about 56 ng/ml, about 57 ng/ml, about 58 ng/ml, about 59 ng/ml, about 60 ng/ml, about 61 ng/ml, about 62 ng/ml, about 63 ng/ml, about 64 ng/ml, about 65 ng/ml, about 66 ng/ml, about 67 ng/ml, about 68 ng/ml, about 69 ng/ml, about 70 ng/ml, about 71 ng/ml, about 72 ng/ml, about 73 ng/ml, about 74 ng/ml, about 75 ng/ml, about 76 ng/ml, about 77 ng/ml, about 78 ng/ml, about 79 ng/ml, about 80 ng/ml, about 81 ng/ml, about 82 ng/ml, about 83 ng/ml, about 84 ng/ml, about 85 ng/ml, about 86 ng/ml, about 87 ng/ml, about 88 ng/ml, about 89 ng/ml, about 90 ng/ml, about 91 ng/ml, about 92 ng/ml, about 93 ng/ml, about 94 ng/ml, about 95 ng/ml, about 96 ng/ml, about 97 ng/ml, about 98 ng/ml, about 99 ng/ml, or about 100 ng/ml.

37-40. (canceled)

41. The method of claim 1, wherein the expanding agent is selected from the group consisting of: IL-2, IL-4, IL-7, IL-9, IL-15, IL-18, IL-21, and any combination thereof, and/or wherein the expanding agent is added in an amount of about 10 U/ml to about 1000 U/ml.

42-47. (canceled)

48. The method of claim 1, wherein the transfection is selected from the group consisting of: a non-viral transfer of one or more nucleic acids encoding an antigen receptor, a chimeric antigen receptor, a T cell receptor, a suicide gene, a gene encoding an inducible caspase 9 system, and any combination thereof into the cell population comprising the Committed CIK Precursor cells in the cell culture, wherein the non-viral transfer of nucleic acids comprises the use of the group consisting of: a transposon-based integration system, Zn-finger nucleases, integrases, transcription activator-like effectors, clustered regularly interspaced short palindromic repeats (CRISPR), sequence-specific recombinase systems able to integrate nucleic acids by recombination between attachment sites, and any combination thereof.

49-51. (canceled)

52. The method of claim 1, wherein one or more nucleic acids encode T cell receptors, chimeric antigen receptors, a cell adhesion molecule, a ligand, a cytokine receptor, a chemokine receptor, a cytokine, a chemokine, an enzyme, or a checkpoint inhibitor.

53. The method of claim 52, wherein one or more nucleic acids encode a prodrug converting enzyme, an IgG-degrading enzyme of S. pyogenes (IdeS), a consensus variant 1 (CV1) protein, Intercellular Adhesion Molecule 1 (ICAM-1), CD137L, OX40L, CD70, IL-15 receptor, CCR5, CCR4, CD25, CD122, CD132, C-X-C chemokine receptor type 4 C-X-C chemokine receptor type 4 (CXCR4), IL-15, IL-18, IL-21 IL-23 IL-33, IL-1a, IL-1b, matrix metalloproteinase (MMP), heparinase, an anti-PD-1 antibody or antigen binding fragment thereof, an anti-T cell immunoglobulin and mucin-domain containing-3 (TIM-3) antibody or antigen binding fragment thereof, IL-3 zetakine, or any combination thereof, or wherein the chimeric antigen receptors are specific for CD19, CD123, TIM-3, C-type lectin-like molecule-1 (CLL-1), CD70, mucin 1 (MUC-1), CD20, CD22, B-cell activating factor receptor (BAFFR), CD23, cytokine receptor like factor 2 (CRLF2), CD79b, CD79d, CD7, CD43, CD5, CD25, Lewis Y (LeY), natural killer group 2 member D (NKG2D), receptor tyrosine kinase like orphan receptor 1 (ROR1), receptor tyrosine kinase like orphan receptor 2 (ROR2), Wilms' tumor 1 (WT1), CD44 variant 6 (CD44v6), CD33, CD3ε, human epidermal growth factor receptor 2 (Her2), epidermal growth factor receptor (EGFR), epidermal growth factor receptor variant III (EGFRvIII), CA125, CD138, prostate-specific membrane antigen (PSMA), B7 homolog 3 protein (B7-H3), CD30, disialoganglioside GD-2, disialoganglioside GD-3, CD171, mesothelin (MSLN), ephrin type-A receptor 2 (EphA2), carcinoembryonic antigen (CEA), vascular endothelial growth factor receptor (VEGFR), IL13Rα2, prostate stem cell antigen (PSCA), epithelial cellular adhesion molecule (EpCAM), chondroitin sulfate proteoglycan 4 (CSPG4), folate receptor alpha (FRα), fibroblast activation protein (FAP), carbonic anhydrase IX (CAIX), B-cell maturation antigen (BCMA), signaling lymphocytic activation molecule family member 7 (SLAM7), CD126, mesenchymal-epithelial transition factor (c-MET), AXL receptor tyrosine kinase (AXL), CD133, tumor endothelial marker 8 (TEM8), tumor-associated calcium signal transducer 2 (TROP2), programmed death-ligand 1 (PD-L1), delta-like ligand 3 (DLL3), melanoma-associated antigen 1 (MAGE-1), protein tyrosine kinase 7 (PTK7), c-type lectin domain containing 14A (CLEC14A), C-X-C chemokine receptor type 4 (CXCR4), New York esophageal squamous cell carcinoma 1 (NY-ESO-1) CD126, tumor-associated glycoprotein 72 (TAG-72), Guanylate cyclase 2C (GUCY2C), Cadherin-6 (CDH6), cadherin 17 (CDH17), claudin18.2 (CLDN18.2), glypican-3 (GPC3), CD147, CD16, glycoprotein 100 (gp100)/human leukocyte antigen-A2 (HLA-A2) complex, MACEP, urokinase-type plasminogen activator receptor (uPAR), alkaline phosphatase, placental-like 2 (ALPPL2), CD47, olfactory receptor 2H1 (OR2H1), matrix metalloproteinase-2 (MMP-2), podoplanin (PDPN), GDNF family receptor alpha-4 (GFRα4), thyrotropin receptor (TSHR), L1 cell adhesion molecule (L1-CAM), or any combination thereof.

54-58. (canceled)

59. The method of claim 1, wherein the Committed CIK Precursor cells are transfected with an RNA encoding SB100X transposase and a DNA encoding a Sleeping Beauty compatible chimeric antigen receptor (CAR) transposon.

60. (canceled)

61. Genetically modified cytokine induced killer (CIK) cells obtained by the method of claim 1.

62. A composition comprising the genetically modified CIK cells of claim 61.

63. (canceled)
